(19) United States
(12) Patent Application Publication
(10) Pub. No.: US 2023/0134843 A1
(43) Pub. Date: May 4, 2023

(54) TREATMENT OF STEM CELL DEFICIENCY

(71) Applicant: OSPEDALE SAN RAFFAELE S.R.L., Milano (MI) (IT)

(72) Inventors: Giulio FERRARI, Milano (MI) (IT); Paolo RAMA, Milano (MI) (IT)

(73) Assignee: OSPEDALE SAN RAFFAELE S.R.L., Milano (MI) (IT)

(21) Appl. No.: 17/910,971

(22) PCT Filed: Mar. 11, 2021
(86) PCT No.: PCT/EP2021/056255

§ 371 Date: Sep. 12, 2022

(30) Foreign Application Priority Data
Mar. 11, 2020 (EP) 20162345.1

(51) Int. Cl. A61K 31/675; A61K 45/06; A61P 43/00

(52) U.S. Cl. CPC A61K 31/675; A61K 45/06; A61P 43/00

(57) ABSTRACT

The present invention refers to an inhibitor of substance P and/or of its receptor for use in the treatment and/or prevention of stem cell deficiency. Preferably the inhibitor is an NK1 antagonist. Preferably the stem cell deficiency is a corneal epithelial stem cell deficiency. The invention also refers to pharmaceutical compositions containing the inhibitor for use in the treatment and/or prevention of stem cell deficiency.

### TECHNICAL FIELD

The present invention refers to an inhibitor of substance P and/or of its receptor pathway for use in the treatment and/or prevention of stem cell deficiency. Preferably the receptor is the receptor neurokinin-1 (NK1). Preferably the inhibitor is an NK1 antagonist. Preferably the stem cell deficiency is a corneal epithelial stem cell deficiency. The inventions also refers to pharmaceutical compositions comprising said inhibitor for use in the treatment and/or prevention of stem cell deficiency.

### BACKGROUND ART

Corneal epithelium serves as a barrier against the environment and its integrity is vital to maintain proper vision. The healthy cornea is densely innervated, mainly by sensory fibers derived from the ophthalmic branch of the trigeminal nerve [1]. Substance P (SP) is an 11-amino-acid polypeptide and it is released by nerve fibers [2], immune cells [3], and epithelial cells [4] in the cornea. SP binds to tachykinin receptors (NK1, NK2, and NK3) expressed on both neuronal and non-neuronal cells [5]. The NK1R (neurokinin-1 receptor) has the highest affinity for SP and its activation has been implicated in promotion of the inflammatory response, corneal neovascularization, and wound healing [6-9].

Corneal epithelial wounds are a common occurrence after ocular trauma or surgery. Prompt closure of corneal ulcers is vital to preserve vision and relies on the capacity of migration, proliferation, and differentiation of limbal stem cells [10-12]. Different molecular pathways are involved in these processes [13]. Among them, the target of rapamycin (mTOR) signaling is a key driver of corneal epithelial cell proliferation and differentiation.

Severe ocular surface diseases can lead to epithelial stem cell deficiency (SCD), which is accompanied by defective healing and ocular surface instability [14]. While epithelial stem cells can be transplanted [15], it is not universally available and its success rate varies depending on techniques used [16,17]. SCD is an area of unmet medical need because it exposes patients to long-term complications such as corneal perforation, infections, and scarring, which may result in permanent vision loss. Therefore, the aim of the present study was to investigate the role of SP in patients affected with SCD and to evaluate the effect of SP/NK1R pathway on epithelium wound healing in a pre-clinical model of corneal stem cell deficiency.

WO 2013/004766 describes NK-1 receptor antagonists and relative pharmaceutical compositions for use in the treatment or prevention of corneal neovascularisation.

WO 98/14193 describes the use of a substance P antagonist for the treatment of ocular pain and compositions for topical ocular use wherein the antagonist has the following formula.

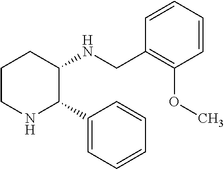 [SMILES: COc1ccccc1CN[C@H]1CCCN[C@H]1c1ccccc1]

There is still the need for effective treatment for stem cell deficiency.

### SUMMARY OF THE INVENTION

The present inventors herein evaluated the role of substance P (SP)/neurokinin-1 receptor (NK1R) pathway in corneal epithelium wound healing in a pre-clinical model of stem cell deficiency (SCD).

In the present invention indeed the expression of substance P (SP) in the tear fluid of patients affected with epithelial stem cell deficiency (SCD) was quantified. Further, the effect of SP ablation on corneal epithelium wound healing in a model of stem cell deficiency was evaluated. SP levels in human tears were measured with a multiplex bead assay.

Eight-week-old C57BL6/N (WT) and B6.Cg-Tac1tm1Bbm/J (TAC1-KO) male mice underwent complete corneal disepithelization, including the limbus. Animals were followed-up for 14 days by quantifying epithelial wound closure and transparency. To test NK1R blockade, some WT mice received NK1R antagonist Fosaprepitant 0.1 and 1 mg/mL topically for 16 days after disepithelization (FOSA 0.1 and FOSA 1). The number of goblet and conjunctival cells infiltrating the cornea, epithelial stem cells, the expression of NK1R, mTOR downstream proteins, and the senescence marker γH2AX were quantified in the cornea.

TAC1-KO mice showed a significant increase in epithelial wound healing rate (p<0.001) and in corneal transparency (p<0.001) up to 14 days after disepithelization, compared to WT. Similarly, FOSA 1 mice showed a significant improvement in wound closure rate and transparency from day five (p<0.05), while FOSA 0.1 group did not show any improvement. TAC1-KO and FOSA 1 mice showed reduced number of infiltrating goblet and conjunctival cells (p<0.05) and increased number of epithelial stem cells (p<0.01), that also expressed NK1R. The mTOR pathway was significantly inhibited (p<0.05) and expression of γH2AX was significantly reduced (p<0.05) in TAC1-KO mice.

The present results suggest that excessive expression of SP is associated with SCD and results in accelerated senescence and exhaustion of residual stem cells, supporting that blockade of SP and/or of its receptors such as neurokinin 1 receptor can be used in the treatment of SCD, in particular as an adjuvant.

The role of Substance P (SP) in a mouse model of corneal epithelial stem cell deficiency was assessed.

Corneal epithelial stem cell deficiency was induced by means of total disepithelization in C57/BL6 (WT) and Substance P knock-out (Tact-KO) mice. In vivo corneal biomicroscopy with fluorescein was performed for up to 2 weeks to quantify epithelial healing and corneal transparency. Upon animal sacrifice, the cornea was stained with Periodic Acid Schiff (PAS) to quantify goblet cells. The inventors used immunostaining for cytokeratin 8 (CTK8) and ABCB5 to quantify conjunctival and epithelial stem cells. The expression of H2A histone family member X (H2AX) and phospho-P70S6 was measured in corneal epithelium of WT vs. Tac1-KO mice with western blot analysis.

Corneal wounds showed improved healing in Tac1-KO as opposed to WT mice (P<0.001). Corneal transparency was significantly higher in Tac1-KO mice (P<0.01). The number of goblet cells was reduced from 2.45±0.39 in WT to 0.77±0.52 in KO and infiltrating conjunctival cells declined from 18.44±1.85 to 6.11±2.98 in KO mice (P<0.04 and P<0.03, respectively). The number of ABCB5+ epithelial stem cells, instead, was higher in KO (19.08±0.46) vs. WT (16.61±0.44) mice (P<0.0001). Finally, the expression of H2AX and phospho-P70S6 was increased in WT vs. KO epithelium (P<0.0277 and P<0.0362, respectively).

The present results indicate that Substance P expression is associated with worse clinical outcome in a pre-clinical model of corneal epithelial stem cell deficiency. Moreover, present data indicate that SP expression can induce stem cell exhaustion by means of DNA damage and m-TOR pathway hyper-activation. These findings have clinical implications, because there are drugs that target Substance P and its receptor.

Then the invention provides an inhibitor of substance P and/or of its receptor for use in the treatment and/or prevention of stem cell deficiency.

Preferably the substance P receptor is the receptor NK1.

Preferably the inhibitor is a NK-1 antagonist, preferably said NK-1 antagonist is Fosaprepitant or a pharmaceutically acceptable salt thereof.

Preferably said inhibitor of the substance P receptor is Fosaprepitant or a pharmaceutically acceptable salt thereof.

Preferably the NK-1 antagonist is administered at a concentration of at least 0.01 mg/ml, preferably at least 10 mg/mL, preferably of at least 20 mg/mL, preferably of at least 30 mg/mL, preferably of at least 40 mg/mL, preferably of at least 50 mg/mL, preferably of at least 60 mg/mL, preferably of at least 100 mg/ml, preferably the NK-1 antagonist is administered once at a concentration of approximately 50 mg/mL, still preferably the NK-1 antagonist is administered between once and six times a day for 1 to 30 days at a concentration of 0.01 to 100 mg/ml.

Preferably the stem cell deficiency is a corneal epithelial stem cell deficiency.

Preferably the stem cell deficiency is selected from the group consisting of: stem cell deficiency induced by aniridia, stem cell deficiency induced by multiple endocrine deficiency, stem cell deficiency induced by epidermal dysplasia such as ectrodactyly-ectodermal dysplasia-clefting syndrome or keratitis-ichthyosis-deafness syndrome, stem cell deficiency induced by erythrokeratodermia, stem cell deficiency induced by dyskeratosis congenita, stem cell deficiency induced by thermal or chemical ocular burn, stem cell deficiency induced by inflammatory eye disease such as Stevens-Johnson syndrome, toxic epidermal necrolysis, ocular cicatricial pemphigoid, chronic limbitis (vernal/atopic conjunctivitis), stem cell deficiency induced by neurotrophic keratopathy, such as trigeminal neuralgia, diabetes, herpes simplex, zoster, stem cell deficiency induced by chronic bullous keratopathy, stem cell deficiency induced by contact lens wear, stem cell deficiency induced by neoplasia, stem cell deficiency induced by extensive limbal cryotherapy, stem cell deficiency induced by irradiation, stem cell deficiency induced by surgery involving the limbus, stem cell deficiency induced by topical antimetabolites (5-FU, mitimycin C), stem cell deficiency induced by systemic chemotherapy, stem cell deficiency induced by microbial infection extending to limbus, stem cell deficiency induced by neurotrophic keratitis, stem cell deficiency induced by vitamin A deficiency, stem cell deficiency induced by Mooren's ulcer, stem cell deficiency induced by malignancy, stem cell deficiency induced by cornea, conjunctiva, ocular surface and lid infections (including trachoma), stem cell deficiency induced by graft versus host disease (GVHD), stem cell deficiency induced by systemic medications including hydroxyurea, stem cell deficiency induced by anterior segment ischaemic syndrome, stem cell deficiency induced by KID (Keratitis Ichthyosis and Deafness syndrome).

Preferably the stem cell deficiency is induced by graft versus host disease.

Preferably the stem cell deficiency is induced by aniridia.

Preferably said inhibitor improves corneal wound healing and/or improves corneal wound closure and/or inhibits at least partially mTOR.

The present invention also provides a pharmaceutical composition comprising the inhibitor as defined above and at least one pharmaceutically acceptable vehicle, for use in the prevention and/or the treatment of stem cell deficiency.

Preferably the inhibitor is Fosaprepitant or a pharmaceutically acceptable salt thereof.

Preferably the pharmaceutical composition comprises 0.01 mg/ml-100 mg/ml of NK-1 antagonist.

Preferably the pharmaceutical composition further comprises at least one agent selected from the group consisting of: an anaesthetic agent, a non-steroidal anti-inflammatory agent, an analgesic agent, an agent useful in the prevention and/or treatment of the disease or condition that causes stem cell deficiency, and an agent that is used following surgery to the eye.

Preferably the composition or the inhibitor is for topical use.

The pharmaceutical composition of the invention may be in the form of an eye-drop formulation. The present invention also provides eye-drops comprising the inhibitor as defined above and at least one pharmaceutically acceptable vehicle, for use in the prevention and/or the treatment of stem cell deficiency.

Preferably, the stem cell deficiency is a corneal epithelial stem cell deficiency.

Preferably, the stem cell deficiency is selected from the group consisting of: stem cell deficiency induced by aniridia, stem cell deficiency induced by multiple endocrine deficiency, stem cell deficiency induced by epidermal dysplasia such as ectrodactyly-ectodermal dysplasia-clefting syndrome or keratitis-ichthyosis-deafness syndrome, stem cell deficiency induced by erythrokeratodermia, stem cell deficiency induced by dyskeratosis congenita, stem cell deficiency induced by thermal or chemical ocular burn, stem cell deficiency induced by inflammatory eye disease such as Stevens-Johnson syndrome, toxic epidermal necrolysis, ocular cicatricial pemphigoid, chronic limbitis (vernal/atopic conjunctivitis), stem cell deficiency induced by neurotrophic keratopathy, such as trigeminal neuralgia, diabetes, herpes simplex, zoster, stem cell deficiency induced by chronic bullous keratopathy, stem cell deficiency induced by contact lens wear, stem cell deficiency induced by neoplasia, stem cell deficiency induced by extensive limbal cryotherapy, stem cell deficiency induced by irradiation, stem cell deficiency induced by surgery involving the limbus, stem cell deficiency induced by topical antimetabolites (5-FU, mitimycin C), stem cell deficiency induced by systemic chemotherapy, stem cell deficiency induced by microbial infection extending to limbus, stem cell deficiency induced by neurotrophic keratitis, stem cell deficiency induced by vitamin A deficiency, stem cell deficiency induced by Mooren's ulcer, stem cell deficiency induced by malignancy, stem cell deficiency induced by cornea, conjunctiva, ocular surface and lid infections (including trachoma), stem cell deficiency induced by graft versus host disease, stem cell deficiency induced by systemic medications including hydroxyurea, stem cell deficiency induced by anterior segment ischaemic syndrome, stem cell deficiency induced by KID (Keratitis Ichthyosis and Deafness syndrome).

More preferably the stem cell deficiency is induced by graft versus host disease or by aniridia.

The present invention also refers to method of the treatment of stem cell deficiency or of all diseases and conditions associated with stem cell deficiency as reported herein, comprising administering an inhibitor of substance P and/or of its receptor to a patient in need thereof. The present invention refers to the treatment of all diseases and conditions associated with stem cell deficiency as reported below, in particular corneal diseases and conditions associated with stem cell deficiency. The present invention refers also to the treatment of stem cell deficiency induced by cornea, conjunctiva, ocular surface and lid infections (including trachoma), graft versus host disease, systemic medications including hydroxyurea, anterior segment ischemic syndrome, KID (Keratitis Ichthyosis and Deafness syndrome).

In the context of the present invention the term “NK-1” and “NK1” or “NK-1R” and “NK1R” may be used interchangeably, and the term “NK-1 antagonist” includes “NK-1R antagonist”.

Examples of diseases and conditions associated with stem cell deficiency are disclosed in the following tables.

TABLE 1

| Primary causes | Secondary causes |
|---|---|
| Aniridia | Thermal or chemical ocular burns* |
| Multiple endocrine | Inflammatory eye disease: |
| deficiency | Stevens-Johnson syndrome, toxic epidermal |
| Epidermal dysplasia | necrolysis |
| Congenital | Ocular cicatricial pemphigoid |
| erythrokeratodermia | Chronic limbitis |
| Dyskeratosis congenita | Neurotrophic keratopathy: |
| | trigeminal neuralgia, diabetes, herpes sim- |
| | plex, zoster |
| | Chronic bullous keratopathy |
| | Contact lens wear* |
| | Neoplasia* |
| | Extensive limbal cryotherapy, irradiation, |
| | surgery* |
| | Topical antimetabolites* |
| | Systemic chemotherapy |

TABLE 2

Hereditary
Anirida
Multiple endocrine de ciency
Epidermal dysplasia
Ectrodactyly-ectodermal dysplasia-clefting syndrome
Keratitis-ichthyosis-deafness (KID) syndrome
Dyskeratosis congenita
Acquired
Chemical or thermal injury
Stevens-Johnson syndrome/toxic epidermal necrolysis
Mucous membrane pemphigoid
Ocular surgeries involving the limbus
Contact lens wear
Severe microbial infection extending to limbus
Antimetabolites (5-FU, mitomycin C)
Radiation
Chronic limbitis (vernal/atopic conjunctivitis)
Bullous keratopathy
Neurotrophic keratitis

TABLE 3

| Nature of | | |
|---|---|---|
| disorder | Limbal stem cell loss | Niche destruction |
| Hereditary | Epidermal dysplasia | Aniridia |
| | | Keratitis secondary to |
| | | endocrine deficiency |
| Acquired | Trauma | Trauma |
| | Chemical and thermal | Antimetabolite use |
| | burns | |
| | Multiple surgeries/ | Radiation |
| | cryotherapy | |
| | Radiation | Local (ocular conditions) |
| | Antimetabolite use | Post-infectious keratitis |
| | Systemic conditions | Neurotrophic keratitis |
| | Stevens-Johnson | Mooren's ulcer |
| | syndrome | |
| | Ocular cicatricial | Peripheral ulcerative |
| | pemphigoid | keratitis |
| | Vitamin A deficiency | Vernal keratoconjunctivitis |
| | Idiopathic | Contact lens use |
| | | Malignancy |

Such diseases or conditions are disclosed in
Zala Lužnik, Marko Hawlina, Stefano Ferrari, Diego Ponzin & Petra Schollmayer (2017) Ocular surface reconstruction in limbal stem cell deficiency: current treatment options and perspectives, Expert Review of Ophthalmology, 12:1, 43-56, DOI: 10.1080/17469899.2017.1263568,
Lal I, Gupta N, Purushotham J, Sangwan V S. Limbal stem cell deficiency: Current management. J Clin Ophthalmol Res 2016; 4:3-12,
Sejpal K, Bakhtiari P, Deng S X. Presentation, diagnosis and management of limbal stem cell deficiency. Middle East Afr J Ophthalmol 2013; 20:5-10,

all herein incorporated by reference.

NK-1 antagonists for use in the present invention may, for example, have an inhibitory concentration (IC50) against the human NK-1 receptor in competition with substance P of less than 100 μM, preferably less than 10 μM, preferably less than 1 μM, preferably less than 100 nM, preferably less than 10 nM, as measured by radiolabeled ligand binding assay on human cells transfected with NK-1 receptor (see for instance Walpole et al, British Journal of Pharmacology (1998); 124:83-92).

NK-1 antagonists are suitably selective antagonists, NK-1 antagonists are suitably selective for NK-1 over other receptors, especially NK-2 and NK-3. Thus NK-1 antagonists may, for example, have an inhibitory concentration (IC50) against the human NK-2 receptor in competition with Neurokinin A which is at least 10 times greater than the inhibitory concentration (IC50) against the human NK-1 receptor in competition with substance P (i.e. it is at least 10 fold selective for NK1 over NK-2), preferably at least 50 fold, preferably at least 100 times selective for NK-1 over NK-2. NK-1 antagonists may, for example, have an inhibitory concentration (IC50) against the human NK-3 receptor in competition with Neurokinin B which is at least 10 times greater than the inhibitory concentration (IC50) against the human NK-1 receptor in competition with substance P (i.e. it is at least 10 fold selective for NK1 over NK-3), preferably at least 50 times, preferably at least 100 fold selective for NK-1 over NK-3.

IC50 values against NK-1, NK-2 and NK-3 receptors may be determined as shown in Walpole et al, supra, by radiolabelled ligand binding assay of human cells transfected with NK1, NK2 or NK3 receptors.

Preferably, the NK-1 antagonist for use in the present invention is selected from the list of NK-1 antagonists disclosed in WO 2013/004766, which is herein incorporated by reference in its entirety.

In particular, the NK-1 antagonist for use in the present invention may be selected from the list consisting of:
a. Aprepitant (MK-0869L-754,030), IUPAC name 5-([(2R,3S)-2-((R)-1-[3,5-bis(trifluoromethyl)phenyl]ethoxy)-3-(4-fluorophenyl)morpholino]methyl)-1H-1,2,4-triazol-3(2H)-one,

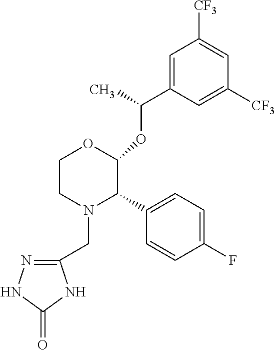 [SMILES: C[C@@H](O[C@H]1OCCN(Cc2n[nH]c(=O)[nH]2)[C@H]1c1ccc(F)cc1)c1cc(C(F)(F)F)cc(C(F)(F)F)c1]

as described and claimed in the following US patents: U.S. Pat. Nos. 5,719,147, 5,538,982, 6,048,859, 6,096,742 and 6,235,735, the contents of which are incorporated herein by reference in their entirety, also described in: Hale J J et al, J Med Chem 1998; 41 (23) 4607-14; as well as pro-drugs thereof, such as:

Fosaprepitant (L-758,298, Emend) IUPAC name [3-{[(2R,3S)-2-[(1R)-1-[3,5-bis(trifluoromethyl)phenyl]ethoxy]-3-(4-fluorophenyl)morpholin-4-yl]methyl}-5-oxo-2H-1,2,4-triazol-1-yl]phosphonic acid

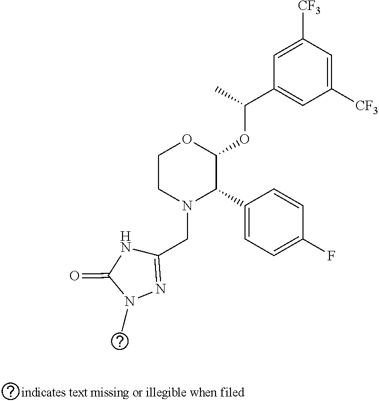 [SMILES: C[C@@H](O[C@H]1OCCN(Cc2nn(C)c(=O)[nH]2)[C@H]1c1ccc(F)cc1)c1cc(C(F)(F)F)cc(C(F)(F)F)c1]

e.g. in the form of a salt such as the dimeglumine salt

as described and claimed at least in U.S. Pat. No. 5,691,336, the contents of which are incorporated herein by reference in its entirety;
b. ZD4974 as described in WO02026724 and WO01077089, the contents of which are incorporated herein by reference in its entirety:

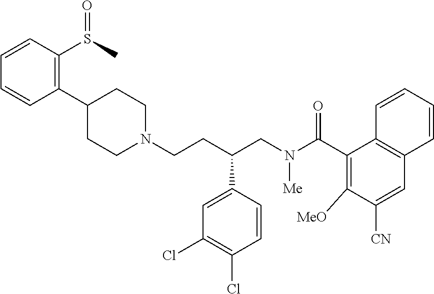 [SMILES: COc1c(C#N)cc2ccccc2c1C(=O)N(C)C[C@@H](CCN1CCC(c2ccccc2[S@](C)=O)CC1)c1ccc(Cl)c(Cl)c1]

c. The following compound, described in WO01077069 and WO00059873, the contents of which are incorporated herein by reference in entirety:

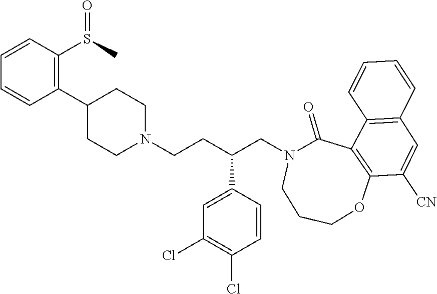 [SMILES: C[S@](=O)c1ccccc1C1CCN(CC[C@H](CN2CCCOc3c(C#N)cc4ccccc4c3C2=O)c2ccc(Cl)c(Cl)c2)CC1]

d. The following compound described in DE19519245, the contents of which are incorporated herein by reference in its entirety:

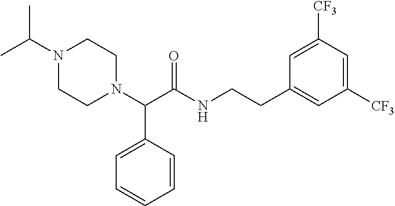 [SMILES: CC(C)N1CCN(C(C(=O)NCCc2cc(C(F)(F)F)cc(C(F)(F)F)c2)c2ccccc2)CC1]

e. The following compound described in WO9732865, the contents of which are incorporated herein by reference in its entirety:

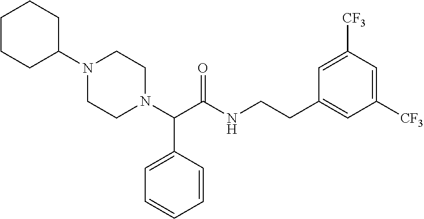 [SMILES: O=C(NCCc1cc(C(F)(F)F)cc(C(F)(F)F)c1)C(c1ccccc1)N1CCN(C2CCCCC2)CC1]

f. The following compound described in EP1295599, the contents of which are incorporated herein by reference in its entirety:

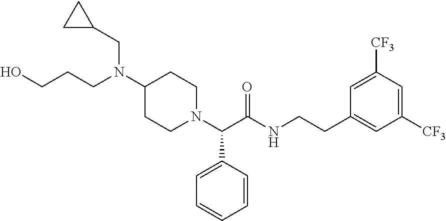 [SMILES: O=C(NCCc1cc(C(F)(F)F)cc(C(F)(F)F)c1)[C@H](c1ccccc1)N1CCC(N(CCCO)CC2CC2)CC1]

g. CGP49823 described in WO9626183 and WO9610562, the contents of which are incorporated herein by reference in their entirety:

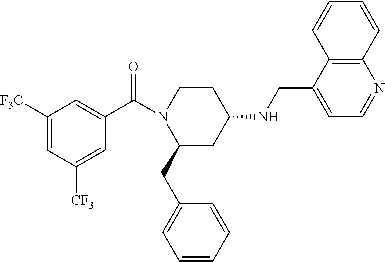 [SMILES: O=C(c1cc(C(F)(F)F)cc(C(F)(F)F)c1)N1CC[C@H](NCc2ccnc3ccccc23)C[C@H]1Cc1ccccc1]

h. The following compound as described in WO9514017, the contents of which are incorporated herein by reference in its entirety:

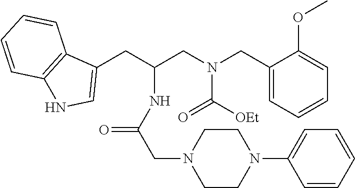 [SMILES: CCOC(=O)N(Cc1ccccc1OC)CC(Cc1c[nH]c2ccccc12)NC(=O)CN1CCN(c2ccccc2)CC1]

i. LY303870, Lanepitant, described in WO9907681, the contents of which are incorporated herein by reference in its entirety:

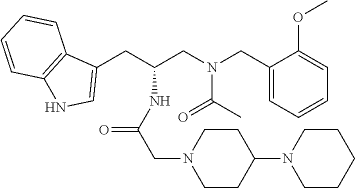 [SMILES: COc1ccccc1CN(C[C@@H](Cc1c[nH]c2ccccc12)NC(=O)CN1CCC(N2CCCCC2)CC1)C(C)=O]

j. LI 686017, described in WO03091226, the contents of which are incorporated herein by reference in its entirety:

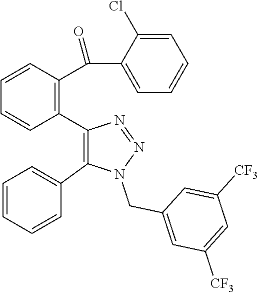 [SMILES: O=C(c1ccccc1Cl)c1ccccc1-c1nnn(Cc2cc(C(F)(F)F)cc(C(F)(F)F)c2)c1-c1ccccc1]

k. FK888, as described in Hagiwara D et al, J Med Chem 1994; 37: 2090-9 and WO9222569, WO93141 13, WO9321215, EP655442 and WO9637488, the contents of which are incorporated herein by reference in their entirety:

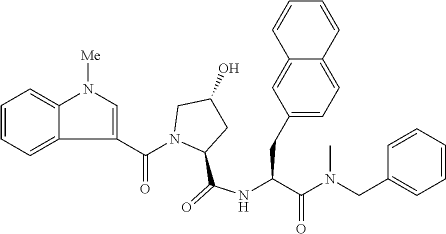 [SMILES: CN(Cc1ccccc1)C(=O)[C@H](Cc1ccc2ccccc2c1)NC(=O)[C@@H]1C[C@@H](O)CN1C(=O)c1cn(C)c2ccccc12]

l. The following compound, described in WO9222569, WO93141 13, WO9321215, EP655442 and WO9637488, the contents of which are incorporated herein by reference in their entirety:

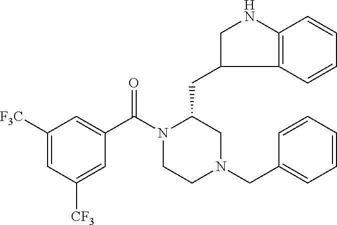 [SMILES: O=C(c1cc(C(F)(F)F)cc(C(F)(F)F)c1)N1CCN(Cc2ccccc2)C[C@H]1CC1CNc2ccccc21]

m. The following compound, described in WO9222569, WO9314113, WO9321215, EP655442 and WO9637488, the contents of which are incorporated herein by reference in their entirety:

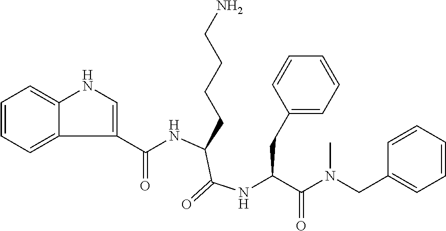 [SMILES: CN(Cc1ccccc1)C(=O)[C@H](Cc1ccccc1)NC(=O)[C@H](CCCCN)NC(=O)c1c[nH]c2ccccc12]

n. The following compound, described in WO00053572, the contents of which are incorporated herein by reference in its entirety:

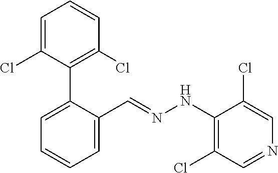 [SMILES: Clc1cncc(Cl)c1N/N=C/c1ccccc1-c1c(Cl)cccc1Cl]

o. Netupitant, described in WO020008232, the contents of which are incorporated herein by reference in its entirety:

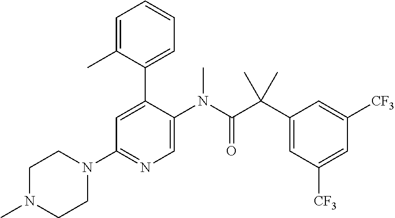 [SMILES: Cc1ccccc1-c1cc(N2CCN(C)CC2)ncc1N(C)C(=O)C(C)(C)c1cc(C(F)(F)F)cc(C(F)(F)F)c1]

p. Befetupitant, described in WO020008232, the contents of which are incorporated herein by reference in its entirety:

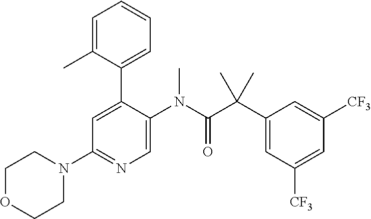 [SMILES: Cc1ccccc1-c1cc(N2CCOCC2)ncc1N(C)C(=O)C(C)(C)c1cc(C(F)(F)F)cc(C(F)(F)F)c1]

q. The following compound, described in WO202062784 and WO020008232, the contents of which are incorporated herein by reference in their entirety:

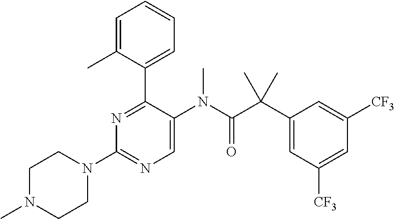 [SMILES: Cc1ccccc1-c1nc(N2CCN(C)CC2)ncc1N(C)C(=O)C(C)(C)c1cc(C(F)(F)F)cc(C(F)(F)F)c1]

r. R116031, described in WO9724356 and WO0716440, the contents of which are incorporated herein by reference in their entirety:

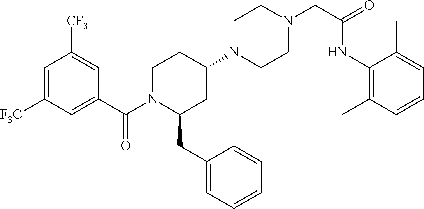 [SMILES: Cc1cccc(C)c1NC(=O)CN1CCN([C@H]2CCN(C(=O)c3cc(C(F)(F)F)cc(C(F)(F)F)c3)[C@H](Cc3ccccc3)C2)CC1]

s. The following compound, described in EP522808, the contents of which are incorporated herein by reference in its entirety:

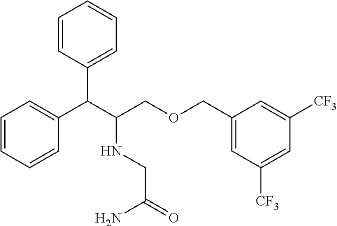 [SMILES: NC(=O)CNC(COCc1cc(C(F)(F)F)cc(C(F)(F)F)c1)C(c1ccccc1)c1ccccc1]

t. The following compound:

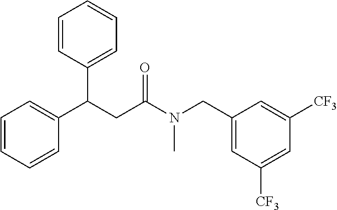 [SMILES: CN(Cc1cc(C(F)(F)F)cc(C(F)(F)F)c1)C(=O)CC(c1ccccc1)c1ccccc1]

u. L733,060

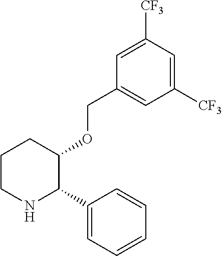 [SMILES: FC(F)(F)c1cc(CO[C@H]2CCCN[C@H]2c2ccccc2)cc(C(F)(F)F)c1]

v. L736,281

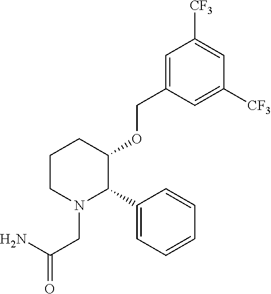 [SMILES: NC(=O)CN1CCC[C@H](OCc2cc(C(F)(F)F)cc(C(F)(F)F)c2)[C@@H]1c1ccccc1]

w. TKA731, described in WO9831704, the contents of which are incorporated herein by reference in its entirety:

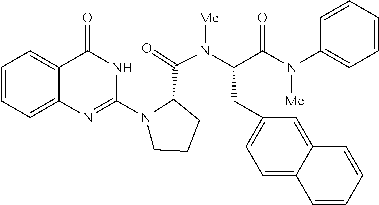 [SMILES: CN(C(=O)[C@H](Cc1ccc2ccccc2c1)N(C)C(=O)[C@@H]1CCCN1c1nc2ccccc2c(=O)[nH]1)c1ccccc1]

x. NKP608, described in WO04024714, the contents of which are incorporated herein by reference in its entirety:

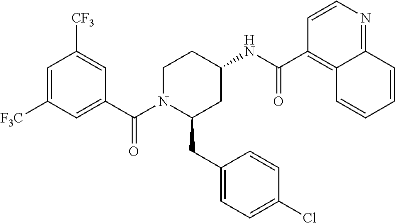 [SMILES: O=C(N[C@H]1CCN(C(=O)c2cc(C(F)(F)F)cc(C(F)(F)F)c2)[C@H](Cc2ccc(Cl)cc2)C1)c1ccnc2ccccc12]

y. CP96,345 described in Lowe J A et al. 1992; 35:2591-600, and in WO92021677, the contents of which are incorporated herein by reference in their entirety;

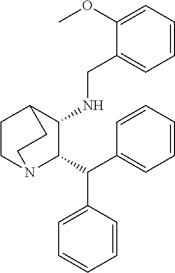 [SMILES: COc1ccccc1CN[C@H]1C2CCN(CC2)[C@H]1C(c1ccccc1)c1ccccc1]

Z. The following compound, described in Lowe J A et al. J Med Chem 1994; 37:2831-40, and in WO92021677, the contents of which are incorporated herein by reference in their entirety;

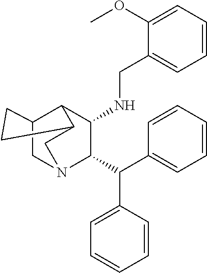 [SMILES: COc1ccccc1CN[C@H]1C2C3CCC2CN(C3)[C@H]1C(c1ccccc1)c1ccccc1]

aa. CP99,994, described in Desai M C et al. J Med Chem 1992; 35:4911-3, the contents of which are incorporated herein by reference in its entirety;

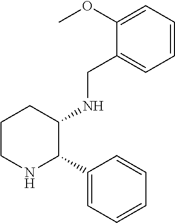 [SMILES: COc1ccccc1CN[C@H]1CCCN[C@H]1c1ccccc1]

bb. CP-122,721 described in Rosen T J et al. Bioorg Med Chem Lett 1998; 8:281-4, the contents of which are incorporated herein by reference in its entirety:

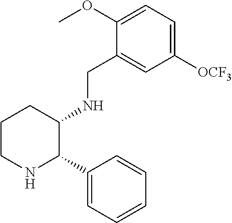 [SMILES: COc1ccc(OC(F)(F)F)cc1CN[C@H]1CCCN[C@H]1c1ccccc1]

cc. CJ-17,493, described in WO9925714, the contents of which are incorporated herein by reference in its entirety:

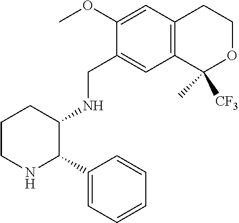 [SMILES: COc1cc2c(cc1CN[C@H]1CCCN[C@H]1c1ccccc1)[C@](C)(C(F)(F)F)OCC2]

dd. Ezlopitant, CJ-1 1,974 described in WO1992021677 the contents of which are incorporated herein by reference in its entirety:

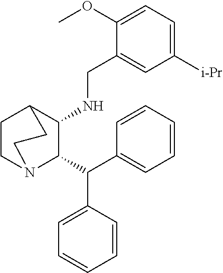 [SMILES: COc1ccc(C(C)C)cc1CN[C@H]1C2CCN(CC2)[C@H]1C(c1ccccc1)c1ccccc1]

ee. Maropitant, CJ-1 1,972, described in WO1992021677, U.S. Pat. Nos. 6,222,038 and 6,255,230, the contents of which are incorporated herein by reference in their entirety:

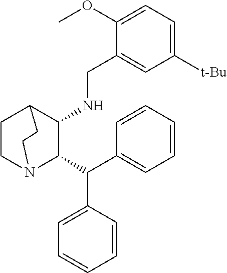 [SMILES: COc1ccc(C(C)(C)C)cc1CN[C@H]1C2CCN(CC2)[C@H]1C(c1ccccc1)c1ccccc1]

ff. RP77580 described in EP429366, the contents of which are incorporated herein reference in its entirety:

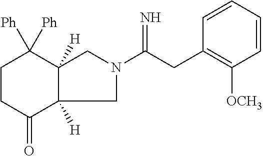 [SMILES: COc1ccccc1CC(=N)N1C[C@H]2C(=O)CCC(c3ccccc3)(c3ccccc3)[C@H]2C1]

gg. Dapitant, RPR100893, described in WO9321154, the contents of which are incorporated herein by reference in its entirety:

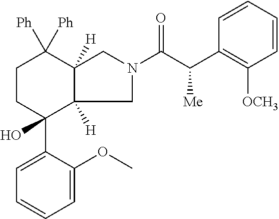 [SMILES: COc1ccccc1[C@H](C)C(=O)N1C[C@@H]2[C@H](C1)C(c1ccccc1)(c1ccccc1)CC[C@@]2(O)c1ccccc1OC]

hh. The following compound, described in EP512901, the contents of which are incorporated herein by reference in its entirety:

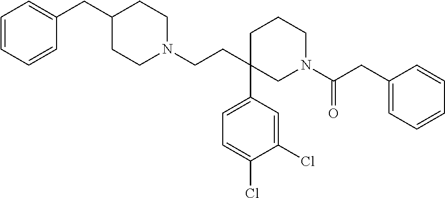 [SMILES: O=C(Cc1ccccc1)N1CCCC(CCN2CCC(Cc3ccccc3)CC2)(c2ccc(Cl)c(Cl)c2)C1]

ii. Nolpitantium, SR140333 described in EP512901, the contents of which are incorporated herein by reference in its entirety:

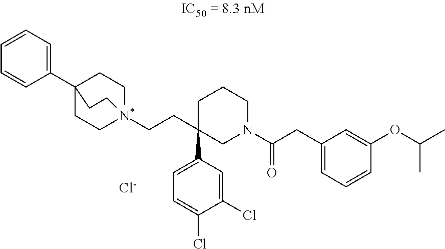

jj. The following compound, described in WO9526338, the contents of which are incorporated herein by reference in its entirety:

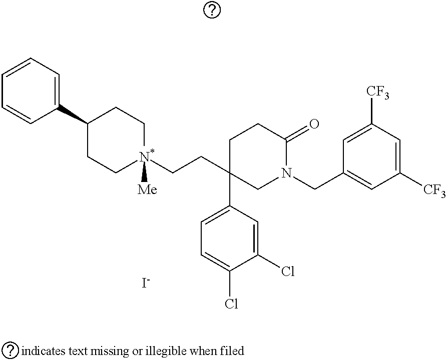

kk. SSR240600, described in WO00068292, the contents of which are incorporated herein by reference in its entirety:

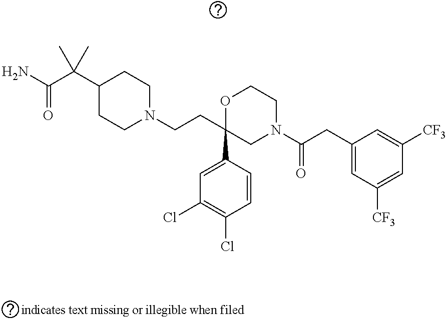 [SMILES: CC(C)(C(N)=O)C1CCN(CC[C@@]2(c3ccc(Cl)c(Cl)c3)CN(C(=O)Cc3cc(C(F)(F)F)cc(C(F)(F)F)c3)CCO2)CC1]

ll. SCH388714 described in WO06065654, the contents of which are incorporated herein by reference in its entirety:

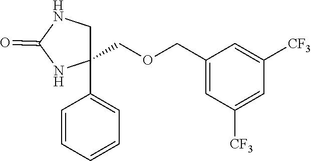 [SMILES: O=C1NC[C@@](COCc2cc(C(F)(F)F)cc(C(F)(F)F)c2)(c2ccccc2)N1]

mm. The following compound described in Paliwal S et al, Bioorg Med Chem Lett 2008; 18:4168-71, the contents of which are incorporated herein by reference in its entirety:

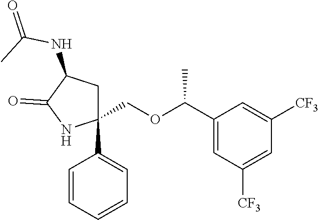 [SMILES: CC(=O)N[C@H]1C[C@@](CO[C@H](C)c2cc(C(F)(F)F)cc(C(F)(F)F)c2)(c2ccccc2)NC1=O]

nn. Rolapitant, described in WO03051840, the contents of which are incorporated herein by reference in its entirety:

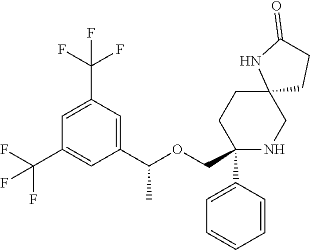 [SMILES: C[C@@H](OC[C@@]1(c2ccccc2)CC[C@]2(CCC(=O)N2)CN1)c1cc(C(F)(F)F)cc(C(F)(F)F)c1]

oo. The following compound, described in EP566069, the contents of which are incorporated herein by reference in its entirety:

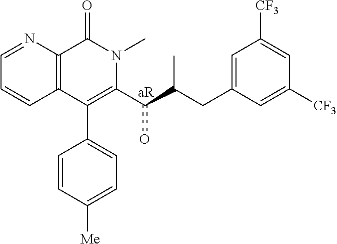

pp. TAK-637, described in JP10259184, the contents of which are incorporated herein by reference in its entirety:

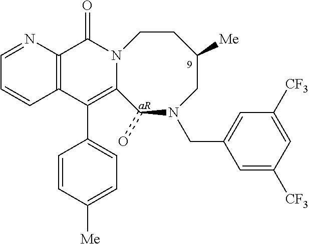

qq. The following compound described in JP2004002334, the contents of which are incorporated herein by reference in its entirety:

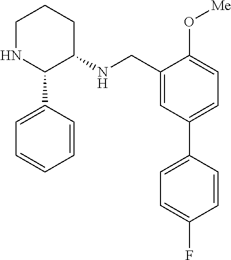 [SMILES: COc1ccc(-c2ccc(F)cc2)cc1CN[C@H]1CCCN[C@H]1c1ccccc1]

rr. The following compound described in JP2007277231 and JP2008239618, the contents of which are incorporated herein by reference in their entirety:

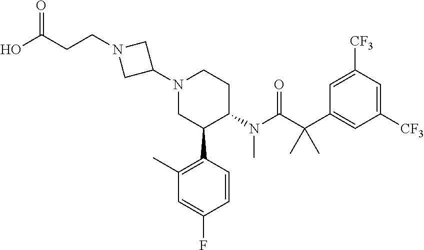 [SMILES: Cc1cc(F)ccc1[C@H]1CN(C2CN(CCC(=O)O)C2)CC[C@@H]1N(C)C(=O)C(C)(C)c1cc(C(F)(F)F)cc(C(F)(F)F)c1]

ss. The following compound described in JP2007277231 and JP2008239618, the contents of which are incorporated herein by reference in their entirety:

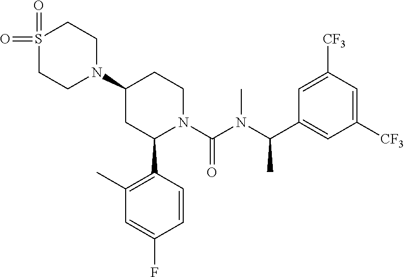 [SMILES: Cc1cc(F)ccc1[C@H]1C[C@@H](N2CCS(=O)(=O)CC2)CCN1C(=O)N(C)[C@H](C)c1cc(C(F)(F)F)cc(C(F)(F)F)c1]

tt. The following compound described in WO9317032 and WO9511686, the contents of which are incorporated herein by reference in their entirety:

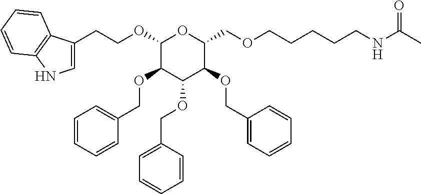 [SMILES: CC(=O)NCCCCCOC[C@H]1O[C@@H](OCCc2c[nH]c3ccccc23)[C@H](OCc2ccccc2)[C@@H](OCc2ccccc2)[C@@H]1OCc1ccccc1]

uu. The following compound described in WO9630367 and WO01025233, the contents of which are incorporated herein by reference in their entirety:

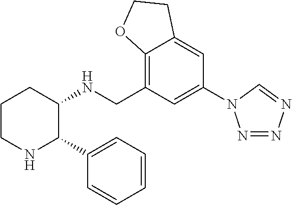 [SMILES: c1ccc([C@@H]2NCCC[C@@H]2NCc2cc(-n3cnnn3)cc3c2OCC3)cc1]

vv. HSP117 described in WO9630367 and WO01025233, the contents of which are incorporated herein by reference in their entirety:

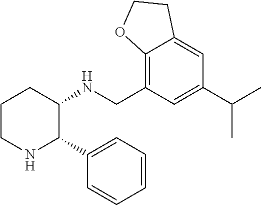 [SMILES: CC(C)c1cc2c(c(CN[C@H]3CCCN[C@H]3c3ccccc3)c1)OCC2]

ww. The following compound, described in Set S, et al. Bioorg Med Chem ILKett 2005; 15:1479-84 and WO03062245, the contents of which are incorporated herein by
reference in their entirety:

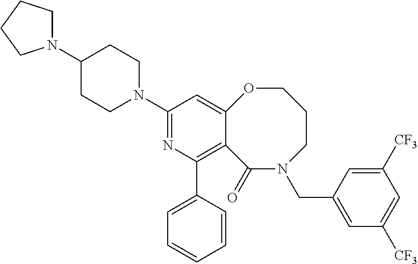 [SMILES: O=C1c2c(cc(N3CCC(N4CCCC4)CC3)nc2-c2ccccc2)OCCCN1Cc1cc(C(F)(F)F)cc(C(F)(F)F)c1]

xx. The following compound, described in Seto S, et al. Bioorg Med Chem Lett 2005; 15:1479-84 and WO03062245, the contents of which are incorporated herein by reference in their entirety:

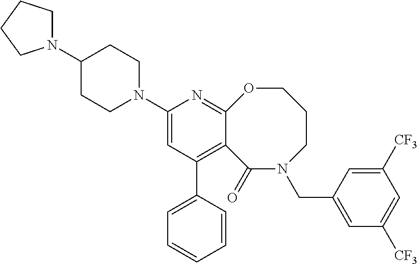 [SMILES: O=C1c2c(-c3ccccc3)cc(N3CCC(N4CCCC4)CC3)nc2OCCCN1Cc1cc(C(F)(F)F)cc(C(F)(F)F)c1]

yy. The following compound, KRP-103, described in WO03062245 and WO05019225, the contents of which are incorporated herein by reference in their entirety:

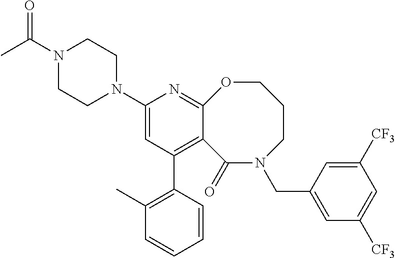 [SMILES: CC(=O)N1CCN(c2cc(-c3ccccc3C)c3c(n2)OCCCN(Cc2cc(C(F)(F)F)cc(C(F)(F)F)c2)C3=O)CC1]

zz. The following compound described in WO06106727, the contents of which are incorporated herein by reference in its entirety:

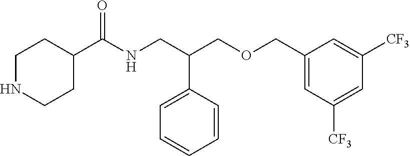 [SMILES: O=C(NCC(COCc1cc(C(F)(F)F)cc(C(F)(F)F)c1)c1ccccc1)C1CCNCC1]

aaa. The following compound, described in WO07074491, the contents of which are incorporated herein by reference in its entirety:

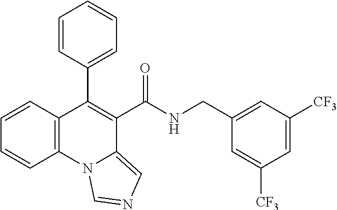 [SMILES: O=C(NCc1cc(C(F)(F)F)cc(C(F)(F)F)c1)c1c(-c2ccccc2)c2ccccc2n2cncc12]

bbb. SLV317, described in US20020065276, the contents of which are incorporated herein by reference in its entirety:

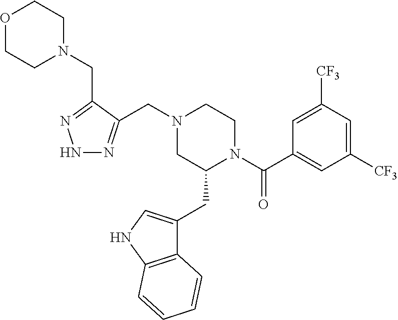 [SMILES: O=C(c1cc(C(F)(F)F)cc(C(F)(F)F)c1)N1CCN(Cc2n[nH]nc2CN2CCOCC2)C[C@H]1Cc1c[nH]c2ccccc12]

ccc. Compounds of formula I described in WO9508549, the contents of which are incorporated herein by reference in its entirety:

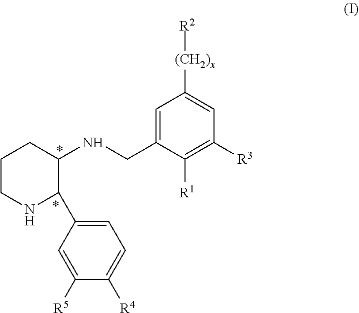

Further examples of NK-1 antagonists include but are not limited to ralopitant e varupitant. Further examples of NK-1 antagonists include but are not limited to the compounds disclosed in the following patent applications, which are incorporated herein by reference in their entirety and to which it is specifically referred to: WO9817660; U.S. Pat. Nos. 5,929,094, 5,877,191, WO00056727, WO04009573, WO00051984, WO01087838, WO02102372, WO02024629, US20050165083, WO06060346, WO06065711, WO07075528, WO06060390, WO07136570 and WO09002770. The NK-1 antagonists according to the invention may optionally be employed in the form of a pharmaceutically acceptable salt including include salts of acidic or basic groups present in NK-1 antagonist compounds of the invention. Pharmaceutically acceptable acid addition salts include, but are not limited to, hydrochloride, hydrobromide, hydroiodide, nitrate, sulfate, bisulfate, phosphate, acid phosphate, isonicotinate, acetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzensulfonate, p-toluenesulfonate, dimeglumine and pamoate salts. Suitable base salts include, but are not limited to, aluminum, calcium, lithium, magnesium, potassium, sodium, zinc, and diethanolamine salts. Pharmaceutically acceptable salts also include hydrates.

An NK-1 Antagonist of the present invention may optionally be provided in the form of a prodrug, i.e. a precursor of a NK1 antagonist that is converted in vivo into an active or more active form (“the parent compound” or “the parent drug”) by metabolic processes or other chemical breakdown event (e.g. hydrolysis). Prodrugs may conveniently be employed in compositions, methods and uses of the invention when they are more soluble than the parent compound. In some embodiments prodrugs of NK-1 antagonists contain one or more phosphate groups not possessed by the parent compound which aid water solubility. For example, a prodrug for use in the present invention is Fosaprepitant, a phosphorylated compound that rapidly converts to Aprepitant following in vivo administration.

Pharmaceutical Compositions and Formulations

The invention also provides a pharmaceutical composition comprising an NK-1 antagonist and a pharmaceutically acceptable vehicle for use in the treatment of stem cell deficiency. Preferably, the pharmaceutical composition of the invention is for topical ocular use and is therefore an ophthalmic composition.

The NK-1 antagonist according to the present invention can be administered by any convenient route, however the preferred route of administration is topically to the ocular surface and specially topically to the cornea.

It is a specific object of the present invention, the use of NK-1 antagonists for the production of an ophthalmic composition to be administered topically to the eye for the therapy and/or prophylaxis of stem cell deficiency.

Accordingly, in a preferred embodiment, the invention provides a method for preventing and treating stem cell deficiency by local administration to the cornea of an ophthalmic composition comprising an NK-1 antagonist.

More generally, one preferred embodiment of the present invention is a composition formulated for topical application on a local, superficial or restricted area in the eye and/or the adnexa of the eye comprising an NK-1 antagonist optionally together with one or more pharmaceutically acceptable additives (such as diluents or carriers).

As used herein, the terms “vehicle”, “diluent”, “carrier” and “additive” are interchangeable.

The ophthalmic compositions of the invention may be in the form of solution, emulsion or suspension (collyrium), ointment, gel, aerosol, mist or liniment together comprising a pharmaceutically acceptable, eye tolerated and compatible with active principle ophthalmic carrier.

Also within the scope of the present invention are particular routes for ophthalmic administration for delayed release, e.g. as ocular erodible inserts or polymeric membrane “reservoir” systems to be located in the conjunctiva sac or in contact lenses.

The ophthalmic compositions of the invention may be administered topically, e.g., the composition is delivered and directly contacts the eye and/or the adnexa of the eye.

The pharmaceutical composition containing at least an NK-1 antagonist of the present invention may be prepared by any conventional technique, e.g. as described in Remington: The Science and Practice of Pharmacy 1995, edited by E. W. Martin, Mack Publishing Company, 19th edition, Easton, Pa.

In one embodiment the composition is formulated so it is a liquid, wherein the NK-1 antagonist may be in solution or in suspension. The composition may be formulated in any liquid form suitable for topical application such as eye-drops, artificial tears, eye washes, or contact lens adsorbents comprising a liquid carrier such as a cellulose ether (e.g. methylcellulose).

Preferably the liquid is an aqueous liquid. It is furthermore preferred that the liquid is sterile. Sterility may be conferred by any conventional method, for example filtration, irradiation or heating or by conducting the manufacturing process under aseptic conditions.

The liquid may comprise one or more lipophile vehicles.

In one embodiment of the present invention, the composition is formulated as an ointment. Preferably one carrier in the ointment may be a petrolatum carrier.

The pharmaceutical acceptable vehicles may in general be any conventionally used pharmaceutical acceptable vehicle, which should be selected according to the specific formulation, intended administration route etc. For example, the pharmaceutical acceptable additives may be any of the additives mentioned in Nema et al, 1997. Furthermore, the pharmaceutical acceptable vehicle may be any accepted additive from FDAs “inactive ingredients list”, which for example is available on the internet address http://www.fda.gov/cder/drug/iig/default.htm.

At least one pharmaceutically acceptable diluents or carrier may be a buffer. For some purposes it is often desirable that the composition comprises a buffer, which is capable of buffering a solution to a pH in the range of 5 to 9, for example pH 5 to 6, pH 6 to 8 or pH 7 to 7.5.

However, in other embodiments of the invention the pharmaceutical composition may comprise no buffer at all or only micromolar amounts of buffer. The buffer may for example be selected from the group consisting of TRIS, acetate, glutamate, lactate, maleate, tartrate, phosphate, citrate, borate, carbonate, glycinate, histidine, glycine, succinate and triethanolamine buffer. Hence, the buffer may be K₂HPO₄, Na₂HPO₄ or sodium citrate.

In a preferred embodiment the buffer is a TRIS buffer. TRIS buffer is known under various other names for example tromethamine including tromethamine USP, THAM, Trizma, Trisamine, Tris amino and trometamol. The designation TRIS covers all the aforementioned designations.

The buffer may furthermore for example be selected from USP compatible buffers for parenteral use, in particular, when the pharmaceutical formulation is for parenteral use. For example the buffer may be selected from the group consisting of monobasic acids such as acetic, benzoic, gluconic, glyceric and lactic, dibasic acids such as aconitic, adipic, ascorbic, carbonic, glutamic, malic, succinic and tartaric, polybasic acids such as citric and phosphoric and bases such as ammonia, diethanolamine, glycine, triethanolamine, and TRIS.

The compositions may contain preservatives such as thimerosal, chlorobutanol, benzalkonium chloride, or chlorhexidine, buffering agents such as phosphates, borates, carbonates and citrates, and thickening agents such as high molecular weight carboxy vinyl polymers such as the ones sold under the name of Carbopol which is a trademark of the B. F. Goodrich Chemical Company, hydroxymethylcellulose and polyvinyl alcohol, all in accordance with the prior art.

In some embodiments of the invention the pharmaceutically acceptable additives comprise a stabiliser. The stabiliser may for example be a detergent, an amino acid, a fatty acid, a polymer, a polyhydric alcohol, a metal ion, a reducing agent, a chelating agent or an antioxidant, however any other suitable stabiliser may also be used with the present invention. For example, the stabiliser may be selected from the group consisting of poloxamers, Tween-20, Tween-40, Tween-60, Tween-80, Brij, metal ions, amino acids, polyethylene glucol, Triton, and ascorbic acid.

Furthermore, the stabiliser may be selected from the group consisting of amino acids such as glycine, alanine, arginine, leucine, glutamic acid and aspartic acid, surfactants such as polysorbate 20, polysorbate 80 and poloxamer 407, fatty acids such as phosphotidyl choline ethanolamine and acethyltryptophanate, polymers such as polyethylene glycol and polyvinylpyrrolidone, polyhydric alcohol such as sorbitol, mannitol, glycerin, sucrose, glucose, propylene glycol, ethylene glycol, lactose and trehalose, antioxidants such as ascorbic acid, cysteine HCL, thioglycerol, thioglycolic acid, thiosorbitol and glutathione, reducing agents such as several thiols, chelating agents such as EDTA salts, gluthamic acid and aspartic acid.

The pharmaceutically acceptable additives may comprise one or more selected from the group consisting of isotonic salts, hypertonic salts, hypotonic salts, buffers and stabilisers.

In preferred embodiments other pharmaceutically excipients such as preservatives are present. In one embodiment said preservative is a parabene, such as but not limited to methyl parahydroxybenzoate or propyl parahydroxybenzoate.

In some embodiments of the invention the pharmaceutically acceptable additives comprise mucolytic agents (for example N-acetyl cysteine), hyaluronic acid, cyclodextrin, petroleum.

Exemplary compounds that may be incorporated in the pharmaceutical composition of the invention to facilitate and expedite transdermal delivery of topical compositions into ocular or adnexal tissues include, but are not limited to, alcohol (ethanol, propanol, and nonanol), fatty alcohol (lauryl alcohol), fatty acid (valeric acid, caproic acid and capric acid), fatty acid ester (isopropyl myristate and isopropyl n-hexanoate), alkyl ester (ethyl acetate and butyl acetate), polyol (propylene glycol, propanedione and hexanetriol), sulfoxide (dimethylsulfoxide and decylmethylsulfoxide), amide (urea, dimethylacetamide and pyrrolidone derivatives), surfactant (sodium lauryl sulfate, cetyltrimethylammonium bromide, polaxamers, spans, tweens, bile salts and lecithin), terpene (d-limonene, alpha-terpeneol, 1,8-cineole and menthone), and alkanone (N-heptane and N-nonane). Moreover, topically-administered compositions may comprise surface adhesion molecule modulating agents including, but not limited to, a cadherin antagonist, a selectin antagonist, and an integrin antagonist.

Also, the ophthalmic solution may contain a thickener such as hydroxymethylcellulose, hydroxyethylcellulose, hydroxypropylmethylcellulose, methylcellulose, polyvinylpyrrolidone, or the like, to improve the retention of the medicament in the conjunctival sac.

In an embodiment, the NK-1 antagonist for use according to the invention may be combined with ophthalmologically acceptable preservatives, surfactants, viscosity enhancers, penetration enhancers, buffers, sodium chloride and water to form aqueous, sterile, ophthalmic suspensions or solutions. The ophthalmic solution may further include an ophthalmologically acceptable surfactant to assist in dissolving the NK-1 antagonist. Ophthalmic solution formulations may be prepared by dissolving the NK-1 antagonist in a physiologically acceptable isotonic aqueous buffer.

In order to prepare sterile ophthalmic ointment formulations, the NK-1 antagonist may be combined with a preservative in an appropriate vehicle, such as, mineral oil, liquid lanolin, or white petrolatum. Sterile ophthalmic gel formulations may be prepared by suspending the NK-1 antagonist in a hydrophilic base prepared from the combination of, for example, carbopol-940, or the like, according to the published formulations for analogous ophthalmic preparations; preservatives and tonicity agents can be incorporated.

Preferably, the formulation of the present invention is An aqueous, nonirritating, ophthalmic composition for topical application to the eye comprising: a therapeutically effective amount of a NK1 antagonist for topical treatment of stem cell deficiency or pharmaceutically acceptable salts thereof; a xanthine derivative being present in an amount between the amount of derivative soluble in the water of said composition and 0.05% by weight/volume of said composition which is effective to reduce the discomfort associated with the NK1 antagonist upon topical application of said composition, said xanthine derivative being selected from the group consisting of theophylline, caffeine, theobromine and mixtures thereof; an ophthalmic preservative; and a buffer, to provide an isotonic, aqueous, nonirritating ophthalmic composition.

Drug Delivery Devices

In one embodiment, the invention comprises a drug-delivery device consisting of at least an NK-1 antagonist and a pharmaceutically compatible polymer. For example, the composition is incorporated into or coated onto said polymer. The composition is either chemically bound or physically entrapped by the polymer. The polymer is either hydrophobic or hydrophilic. The polymer device comprises multiple physical arrangements. Exemplary physical forms of the polymer device include, but are not limited to, a film, a scaffold, a chamber, a sphere, a microsphere, a stent, or other structure. The polymer device has internal and external surfaces. The device has one or more internal chambers. These chambers contain one or more compositions. The device contains polymers of one or more chemically-differentiable monomers. The subunits or monomers of the device polymerize in vitro or in vivo.

In a preferred embodiment, the invention comprises a device comprising a polymer and a bioactive composition incorporated into or onto said polymer, wherein said composition includes an NK-1 antagonist, and wherein said device is implanted or injected into an ocular surface tissue, an adnexal tissue in contact with an ocular surface tissue, a fluid-filled ocular or adnexal cavity, or an ocular or adnexal cavity.

Exemplary mucoadhesive polyanionic natural or semi-synthethic polymers from which the device may be formed include, but are not limited to, polygalacturonic acid, hyaluronic acid, carboxymethylamylose, carboxymethylchitin, chondroitin sulfate, heparin sulfate, and mesoglycan. In one embodiment, the device comprises a biocompatible polymer matrix that may optionally be biodegradable in whole or in part. A hydrogel is one example of a suitable polymer matrix material. Examples of materials which can form hydrogels include polylactic acid, polyglycolic acid, PLGA polymers, alginates and alginate derivatives, gelatin, collagen, agarose, natural and synthetic polysaccharides, polyamino acids such as polypeptides particularly poly(lysine), polyesters such as polyhydroxybutyrate and poly-.epsilon.-caprolactone, polyanhydrides; polyphosphazines, polyvinyl alcohols), poly(alkylene oxides) particularly poly(ethylene oxides), poly(allylamines)(PAM), poly(acrylates), modified styrene polymers such as poly(4-aminomethylstyrene), pluronic polyols, polyoxamers, poly(uronic acids), poly(vinylpyrrolidone) and copolymers of the above, including graft copolymers. In another embodiment, the scaffolds may be fabricated from a variety of synthetic polymers and naturally-occurring polymers such as, but not limited to, collagen, fibrin, hyaluronic acid, agarose, and laminin-rich gels.

One preferred material for the hydrogel is alginate or modified alginate material. Alginate molecules are comprised of (1-4)-linked β-D-mannuronic acid (M units) and a L-guluronic acid (G units) monomers which vary in proportion and sequential distribution along the polymer chain. Alginate polysaccharides are polyelectrolyte systems which have a strong affinity for divalent cations (e.g. Ca⁺², Mg⁺², Ba⁺²) and form stable hydrogels when exposed to these molecules. See Martinsen A., et al., Biotech. & Bioeng, 33 (1989) 79-89.

The device is administered topically, subconjunctively, or in the episcleral space, subcutaneously, or intraductally. Specifically, the device is placed on or just below the surface of an ocular tissue. Alternatively, the device is placed inside a tear duct or gland. The composition incorporated into or onto the polymer is released or diffuses from the device.

In one embodiment the composition is incorporated into or coated onto a contact lens or drug delivery device, from which one or more molecules diffuse away from the lens or device or are released in a temporally-controlled manner. In this embodiment, the contact lens composition either remains on the ocular surface, e.g. if the lens is required for vision correction, or the contact lens dissolves as a function of time simultaneously releasing the composition into closely juxtaposed tissues. Similarly, the drug delivery device is optionally biodegradable or permanent in various embodiments.

For example, the composition is incorporated into or coated onto said lens. The composition is chemically bound or physically entrapped by the contact lens polymer. Alternatively, a colour additive is chemically bound or physically entrapped by the polymer composition that is released at the same rate as the therapeutic drug composition, such that changes in the intensity of the colour additive indicate changes in the amount or dose of therapeutic drug composition remaining bound or entrapped within the polymer. Alternatively, or in addition, an ultraviolet (UV) absorber is chemically bound or physically entrapped within the contact lens polymer. The contact lens is either hydrophobic or hydrophilic.

Exemplary materials used to fabricate a hydrophobic lens with means to deliver the compositions of the invention include, but are not limited to, amefocon A, amsilfocon A, aquilafocon A, arfocon A, cabufocon A, cabufocon B, carbosilfocon A, crilfocon A, crilfocon B, dimefocon A, enflufocon A, enflofocon B, erifocon A, flurofocon A, flusilfocon A, flusilfocon B, flusilfocon C, flusilfocon D, flusilfocon E, hexafocon A, hofocon A, hybufocon A, itabisfluorofocon A, itafluorofocon A, itafocon A, itafocon B, kolfocon A, kolfocon B, kolfocon C, kolfocon D, lotifocon A, lotifocon B, lotifocon C, melafocon A, migafocon A, nefocon A, nefocon B, nefocon C, onsifocon A, oprifocon A, oxyfluflocon A, paflufocon B, paflufocon C, paflufocon D, paflufocon E, paflufocon F, pasifocon A, pasifocon B, pasifocon C, pasifocon D, pasifocon E, pemufocon A, porofocon A, porofocon B, roflufocon A, roflufocon B, roflufocon C, roflufocon D, roflufocon E, rosilfocon A, satafocon A, siflufocon A, silafocon A, sterafocon A, sulfocon A, sulfocon B, telafocon A, tisilfocon A, tolofocon A, trifocon A, unifocon A, vinafocon A, and wilofocon A. Exemplary materials used to fabricate a hydrophilic lens with means to deliver the compositions of the invention include, but are not limited to, abafilcon A, acofilcon A, acofilcon B, acquafilcon A, alofilcon A, alphafilcon A, amfilcon A, astifilcon A, atlafilcon A, balafilcon A, bisfilcon A, bufilcon A, comfilcon A, crofilcon A, cyclofilcon A, darfilcon A, deltafilcon A, deltafilcon B, dimefilcon A, droxfilcon A, elastofilcon A, epsilfilcon A, esterifilcon A, etafilcon A, focofilcon A, galyfilcon A, genfilcon A, govafilcon A, hefilcon A, hefilcon B, hefilcon C, hilafilcon A, hilafilcon B, hioxifilcon A, hioxifilcon B, hioxifilcon C, hydrofilcon A, lenefilcon A, licryfilcon A, licryfilcon B, lidofilcon A, lidofilcon B, lotrafilcon A, lotrafilcon B, mafilcon A, mesafilcon A, methafilcon B, mipafilcon A, nelfilcon A, netrafilcon A, ocufilcon A, ocufilcon B, C, ocufilcon D, ocufilcon E, ofilcon A, omafilcon A, oxyfilcon A, pentafilcon A, perfilcon A, pevafilcon A, phemfilcon A, polymacon, senofilcon A, silafilcon A, siloxyfilcon A, surfilcon A, tefilcon A, tetrafilcon A, trilfilcon A, vifilcon A, vifilcon B, and xylofilcon A.

Within the scope of the invention are compositions formulated as a gel or gel-like substance, creme or viscous emulsions. It is preferred that said compositions comprise at least one gelling component, polymer or other suitable agent to enhance the viscosity of the composition. Any gelling component known to a person skilled in the art, which has no detrimental effect on the area being treated and is applicable in the formulation of compositions and pharmaceutical compositions for topical administration to the skin, eye or mucous can be used. For example, the gelling component may be selected from the group of: acrylic acids, carbomer, carboxypolymethylene, such materials sold by B. F. Goodrich under the trademark Carbopol (e.g. Carbopol 940), polyethylene-polypropyleneglycols, such materials sold by BASF under the trademark Poloxamer (e.g. Poloxamer 188), a cellulose derivative, for example hydroxypropyl cellulose, hydroxyethyl cellulose, hydroxyethylene cellulose, methyl cellulose, carboxymethyl cellulose, alginic acid-propylene glycol ester, polyvinylpyrrolidone, veegum (magnesium aluminum silicate), Pemulen, Simulgel (such as Simulgel 600, Simulgel EG, and simulgel NS), Capigel, Colafax, plasdones and the like and mixtures thereof.

A gel or gel-like substance according to the present invention comprises for example less than 10% w/w water, for example less than 20% w/w water, for example at least 20% w/w water, such as at least 30% w/w water, for example at least 40% w/w water, such as at least 50% w/w water, for example at least 75% w/w water, such as at least 90% w/w water, for example at least 95% w/w water. Preferably said water is deionised water.

Gel-like substances of the invention include a hydrogel, a colloidal gel formed as a dispersion in water or other aqueous medium. Thus a hydrogel is formed upon formation of a colloid in which a dispersed phase (the colloid) has combined with a continuous phase (i.e. water) to produce a viscous jellylike product; for example, coagulated silicic acid. A hydrogel is a three-dimensional network of hydrophilic polymer chains that are crosslinked through either chemical or physical bonding. Because of the hydrophilic nature of the polymer chains, hydrogels absorb water and swell. The swelling process is the same as the dissolution of non-crosslinked hydrophilic polymers. By definition, water constitutes at least 10% of the total weight (or volume) of a hydrogel.

Examples of hydrogels include synthetic polymers such as polyhydroxy ethyl methacrylate, and chemically or physically crosslinked polyvinyl alcohol, polyacrylamide, poly(N-vinyl pyrrolidone), polyethylene oxide, and hydrolyzed polyacrylonithle. Examples of hydrogels which are organic polymers include covalent or ionically crosslinked polysacchande-based hydrogels such as the polyvalent metal salts of alginate, pectin, carboxymethyl cellulose, heparin, hyaluronate and hydrogels from chitin, chitosan, pullulan, gellan and xanthan. The particular hydrogels used in our experiment were a cellulose compound (i.e. hydroxypropylmethylcellulose [HPMC]) and a high molecular weight hyaluronic acid (HA).

Hyaluronic acid is a polysaccharide made by various body tissues. U.S. Pat. No. 5,166,331 discusses purification of different fractions of hyaluronic acid for use as a substitute for intraocular fluids and as a topical ophthalmic drug carrier. Other U.S. patent applications which discuss ocular uses of hyaluronic acid include Ser. Nos. 11/859,627; 11/952,927; 10/966,764; 11/741,366; and 11/039,192 Formulations of macromolecules for intraocular use are known, See eg U.S. patent application Ser. Nos. 11/370,301; 11/364,687; 60/721,600; 11/116,698 and 60/567,423; 11/695,527. Use of various active agents is a high viscosity hyaluronic acid is known. See eg U.S. patent application Ser. Nos. 10/966,764; 11/091,977; 11/354,415; 60/519,237; 60/530,062, and; Ser. No. 11/695,527.

Sustained release formulations as described in WO2010048086 are within the scope if the invention.

Concentration of Active Ingredient and Therapeutic Regimen

Compositions and pharmaceutical compositions according to the present invention, comprise at least one NK-1 antagonist as an active ingredient. The concentration of NK-1 antagonist in said compositions may vary according to the type of administration they are formulated for. The compositions may comprise 0.01 mg/ml to 100 mg/ml, preferably 0.01 mg/ml to 10 mg/ml, such as 100 ng/ml to 10 mg/ml, preferably 1 mg/ml to 10 mg/ml NK-1 antagonist.

In a preferred embodiment, pharmaceutical compositions according to the present invention comprise at least 10 mg/ml of active ingredient. In another preferred embodiment, pharmaceutical compositions according to the present invention comprise at least 50 mg/ml of active ingredient.

Accordingly, the total dose per day of active principle may comprise 10 ng to 100 mg, preferably 100 ng to 10 mg, preferably 10 μg to 10 mg, preferably 200 μg to 1 mg, preferably 200 μg, of NK-1 antagonist. In a preferred embodiment, the total dose per day of active principle is of at least 50 mg, preferably of at least 60 mg.

The compositions may comprise 0.01 to 50% (weight/volume) of NK-1 Antagonist, preferably 0.05 to 5% (weight/volume), more preferably 0.05 to 1 wt % (weight/volume), or most preferably 0.1 to 2% (weight/volume) of the NK-1 Antagonist, for example the composition may comprise 0.05% (weight/volume), 0,075% (weight/volume), 0.1% (weight/volume), 1%, (weight/volume), 2% (weight/volume, 40% (weight/volume), 5% (weight/volume), of NK-1 antagonist.

According to the present invention “a therapeutically effective amount” of the composition refers to the amount necessary to induce the desired biological effect on the subject in need of treatment.

The compositions and pharmaceutical compositions according to the present invention may be administered once or several times per day, for example they may be administered in the range of 1 to 10 times a day, such as e.g. 1 to 8 times, for example 1 to 6 times, such as 1 to 4 times, such as 1 to 3 times a day. Preferably, the NK-1 antagonists and the pharmaceutical compositions according to the present invention are administered six times a day.

The compositions according to the present invention may be administrated to the subject for a period of treatment of one or more than one week such as two weeks, three weeks, four weeks, five weeks, six weeks, seven weeks, eight weeks or more than eight weeks. The treatment may be repeated on subjects who relapse.

Advantageously, the NK-1 antagonists and the pharmaceutical compositions according to the present invention may be administered to the subject only once using the above-defined dosages.

A further aspect of the present invention relates to a method of treating or ameliorating a medical condition of the eye characterized by the presence of stem cell deficiency comprising administration to an animal subject including a human being in need thereof an effective dosage of a composition or a pharmaceutical composition as defined herein above.

Combination Therapy

In one embodiment the treatment or prevention of stem cell deficiency consists of the use of an NK-1 antagonist as sole pharmaceutically active agent.

However in certain embodiments the invention further encompasses the administration of an NK-1 antagonist concurrently with one or more further therapeutically active agents that are administered to the same patient, each active agent being administered according to a regimen suitable for that medicament. This encompasses pre-treatment, simultaneous treatment, sequential treatment, and alternating regimens.

The one or more therapeutically active agents may be administered by the same route as the NK-1 antagonist or by a different route (or by one or more different routes).

At least one of the one or more further therapeutically active agents may, for example, administered topically to the eye.

Examples of such active agents include but are not limited to antivirals, antibacterial agents (such as antibiotics), analgesics, antagonists of inflammatory cytokines, corticosteroids, non-steroidal anti-inflammatory agents, immunosuppressants, anti-fungal agents and anesthetics. Preferably, the one or more further therapeutically active agent may be an agent that is useful in the prevention and/or treatment of stem cell deficiency, such as an anesthetic agent, a non-steroidal anti-inflammatory agent or an analgesic. Still preferably, the one or more further therapeutically active agent may be an agent that is useful in the prevention and/or treatment of stem cell deficiency or an agent that is used following surgery to the eye. In one specific embodiment, the invention encompasses a method of treating or preventing stem cell deficiency by administering an NK-1 antagonist concurrently with an antibiotic agent.

In one specific embodiment there is provided a pharmaceutical composition suitable for topical administration to the eye comprising an NK-1 antagonist and an antibiotic agent.

Typically such a composition will comprise one or more diluents or carriers which are pharmaceutically acceptable for topical administration to the eye.

In another embodiment, the one or more further therapeutically active agents are selected from VEGF inhibitors, IL1-R inhibitors, immunosuppressants and TNF inhibitors.

In one embodiment of the invention, one of the one or more further therapeutically active agents is an antibiotic such as amikacin, gentamicin, kanamycin, neomycin, netilmicin, streptomycin, tobramycin, teicoplanin, vancomycin, azithromycin, clarithromycin, clarithromycin, dirithromycin, erythromycin, roxithromycin, troleandomycin, amoxicillin, ampicillin, azlocillin, carbenicillin, clozacillin, dicloxacillin, flucozacillin, mezlocillin, nafcillin, penicillin, piperacillin, ticarcillin, bacitracin, colistin, polymyxin B, ciprofloxacin, enoxacin, gatifloxacin, levofloxacin, lomefloxacin, moxifloxacin, norfloxacin, oflazacin, trovafloxacin, mafenide, sulfacetamide, sulfamethizole, sulfasalazine, sulfisoxazole, tetracycline, trimethoprim, cotrimoxazole, demeclocycline, soxycycline, minocycline, doxycycline, oxytetracycline or tetracycline.

In a further embodiment of the invention, one of the one or more further therapeutically active agents is an immunosuppressive agent such as cyclosporin A.

In a further embodiment of the invention, one of the one or more further therapeutically active agents is an antagonist of inflammatory cytokines such as antagonist of tumor necrosis factor alpha (TNFα). Exemplary functional blockers of TNFα include, but are not limited to, recombinant and/or soluble TNFα receptors, monoclonal antibodies, and small molecule antagonists and/or inverse agonists. One or more commercially-available TNF-α blocking agents are reformulated for topical administration in this embodiment. Exemplary commercial TNF-α blocking agents used for reformulation include, but are not limited to, etanerept/Embrel, infliximab/Remicade, and adalimumab/Humira.

Alternatively, one of the one or more further therapeutically active agents is an antagonist of an inflammatory cytokine selected from IL-I, IL-2, IL-4, IL-5, IL-6, IL-8, IL-12, IL-17, IL-18 and IL-23.

In a further embodiment of the invention, one of the one or more further therapeutically active agents is an antagonist of one or more member(s) of the vascular epithelial growth factor (VEGF) family. Exemplary members include, but are not limited to, VEGF-A, VEGF-C, VEGFR-2, and VEGFR-3. Anti-VEGF agents which inhibit either VEGF itself or the VEGF receptor present in the eye in order to thereby prevent angiogenesis, include but are not limited to monoclonal antibodies such as ranibizumab (LUCENTIS®; rhuFab V2) and bevacizumab (AVASTIN®; rhuMab-VEGF), nucleic acids (aptamers such as MACUGEN®, (pegaptanib) a PEGylated RNA aptamer, and siRNAs directed to VEGF RNA). Bevacizumab is a full-length anti-VEGF antibody approved for use in metastatic colon cancer. Ranibizumab is a humanized anti-VEGF monoclonal antibody fragment that inhibits all isotypes of VEGF and pegaptanib is a VEGF-neutralizing aptamer that specifically inhibits one isoform of VEGF (VEGF-165).

Further examples include antibody fragments (e.g. Ranibizumab), small interfering RNA's decreasing expression of VEGFR or VEGF ligand, post-VEGFR blockade with tyrosine kinase inhibitors, Small molecule RTK inhibitors targeting VEGF receptors including PTK787 can also be used.

In a further embodiment of the invention, one of the one or more further therapeutically active agents is an antagonist of interferon-gamma.

In a further embodiment of the invention, one of the one or more further therapeutically active agents is an antagonist of one or more chemokines and their receptors. Exemplary chemokines and receptors that may be antagonized by a further active agent include chemokine (C-C motif) receptor 1 (CCRI), chemokine (C-C motif) receptor 2 (CCR2), chemokine (C-C motif) receptor 5 (CCR5), chemokine (C-C motif) receptor 7 (CCR7), and chemokine (C-X-C motif) receptor 3 (CXC R3).

The invention will be illustrated by means of non-limiting examples in reference to the following figures.

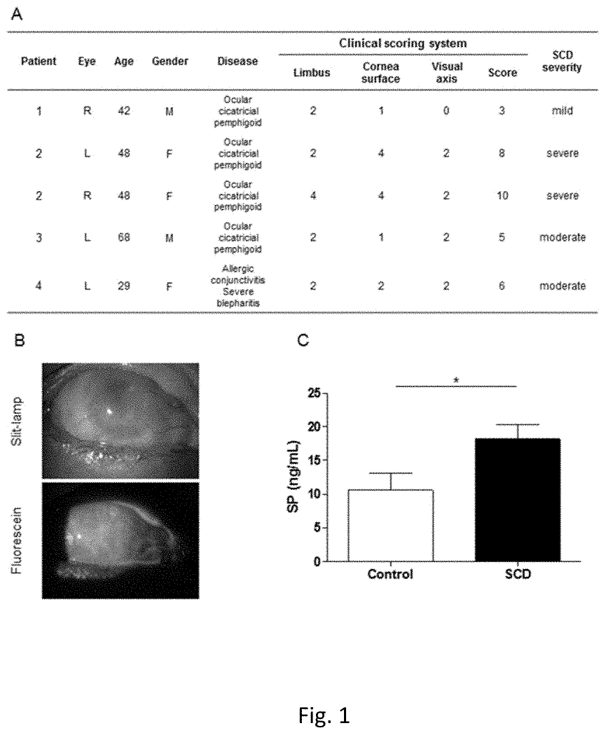

FIG. 1. SP levels in tears are increased in SCD-affected patients. (A) Demographics of the SCD patients enrolled in this study. (B) Slit-lamp and fluorescein representative pictures of patient no 2 right eye, where the great extent of limbus and corneal involvement can be observed. (C) Tears SP levels of SCD-patients and age-matched controls.

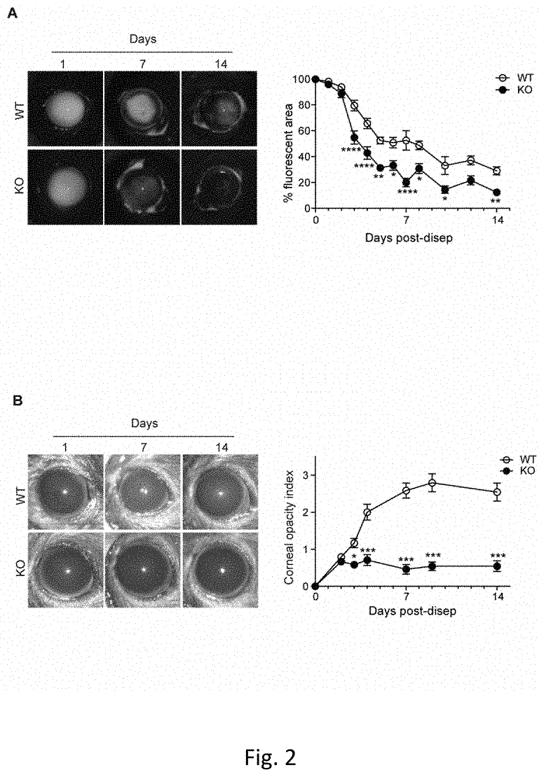

FIG. 2. Substance P ablation improves corneal epithelial wound healing and corneal transparency in a stem cell deficiency model. (A) Stem cell deficiency model. Wound healing progress evaluated by fluorescein assay. When compared to WT, TAC1-KO mice show significant faster epithelial wound healing, starting from the third day after disepithelization. (B) Corneal opacity assay. TAC1-KO mice show a significant decrease in corneal opacity compared to WT mice. Graphs show mean values ±SEM; statistical analysis by unpaired Student's t-test *p<0.05 **p<0.01 ***p<0.001 ****p<0.0001

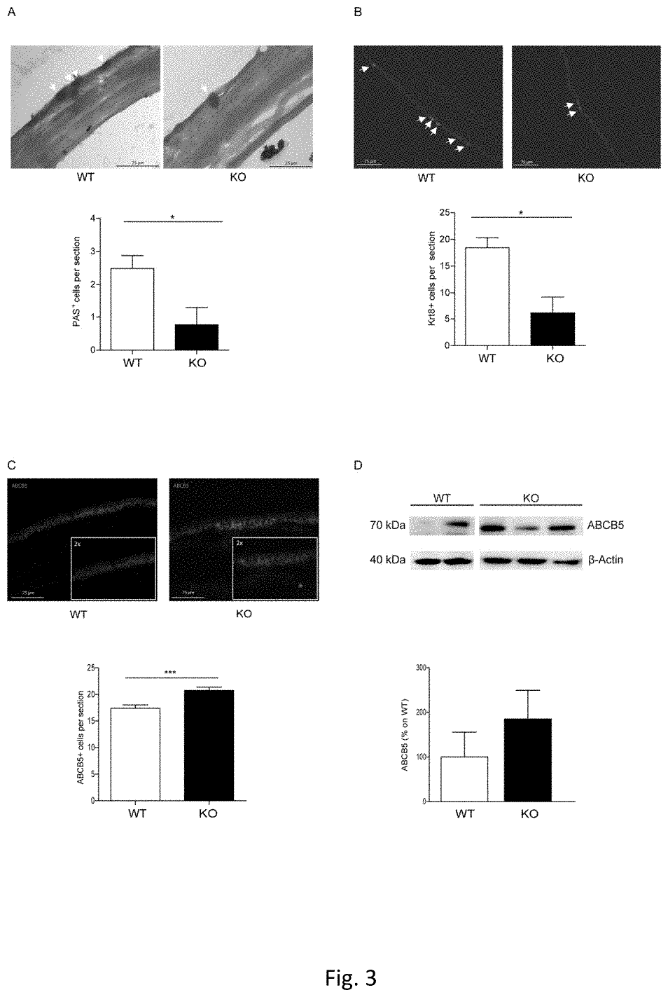

FIG. 3. Substance P ablation reduces the number of goblet and conjunctival cells infiltrating the cornea, and alleviates stem cell deficiency. (A) PAS staining for goblet cells identification in the cornea. Representative images are shown (40×). Reduced number of PAS⁺ cells is observed in TAC1-KO mice compared to WT. (B) Keratin 8 (Krt8) staining for conjunctival cells identification. Representative images of the immunofluorescence are shown (10×). Reduced number of Krt8⁺ conjunctival cells is observed in TAC1-KO compared to WT. (C) ABCB5 expression as a stem cell marker evaluated by immunofluorescence. Representative images are shown (20×). When compared to WT, TAC1-KO mice exhibit an increase in ABCB5 expression in corneal epithelium. (D) ABCB5 expression assessed by western blot. Graphs show mean values ±SEM; statistical analysis by unpaired Student's t-test *p<0.05

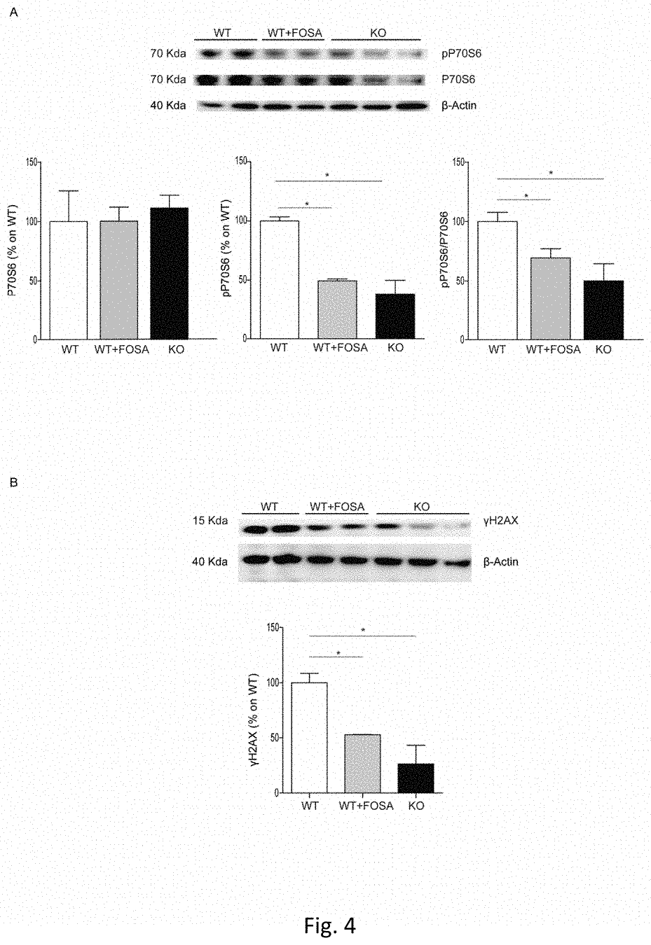

FIG. 4. Substance P ablation and its pharmacological inhibition result in decreased mTOR signaling and senescence in corneal epithelium. (A) Expression of P70S6 and pP70S6, downstream proteins in mTOR pathway, evaluated by western blot. A representative blot is shown. When compared to WT, TAC1-KO and WT-FOSA mice show a significant decrease in pP70S6 expression. The pP70S6/P70S6 expression ratio was calculated in order to determine mTOR pathway activation. A significant decrease in this ratio is observed in TAC1-KO and WT-FOSA mice, compared to WT, suggesting reduced mTOR pathway activation. No differences were found between WT-FOSA and TAC1-KO in P70S6 or pP70S6 expression. (B) Expression of H2AX as a senescence marker. A representative blot is shown. TAC1-KO and WT-FOSA mice exhibit a decreased H2AX expression in comparison to WT. Graphs show mean values ±SEM; statistical analysis by unpaired Student's t-test *p<0.05.

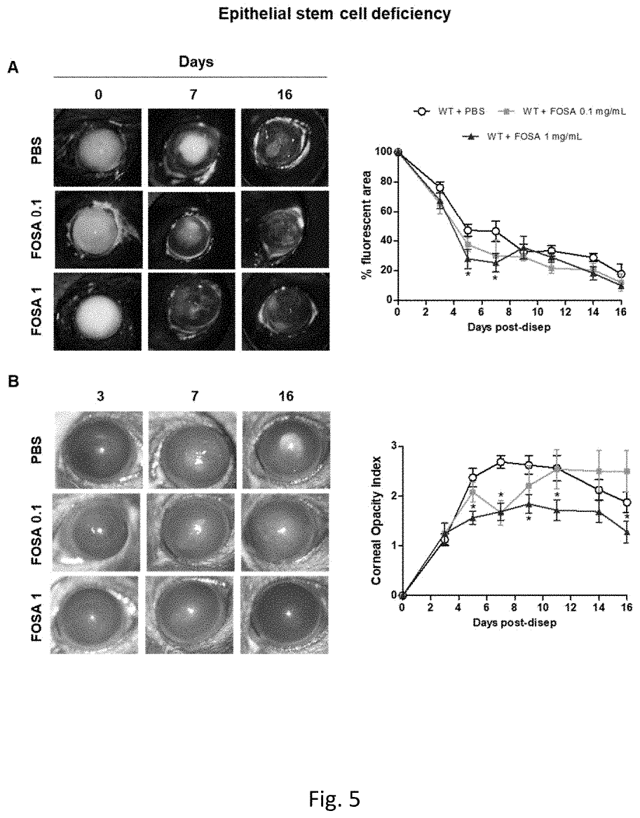

FIG. 5. Neurokinin-1 receptor blockade promotes epithelial wound healing and corneal transparency in a stem cell deficiency model. A—Wound healing progress evaluated by fluorescein assay. When compared to WT, mice topically receiving Fosaprepitant 1 mg/mL (FOSA 1) displayed a significant faster epithelial wound closure, meanwhile the application of Fosaprepitant 0.1 mg/mL (FOSA 0.1) did not show any improvement. B—Corneal opacity evaluation. FOSA 1 mice showed a significant improvement in corneal transparency compared to WT mice. Graphs show mean values ±SEM; statistical analysis by One-way ANOVA followed by Tukey's test. *p<0.05

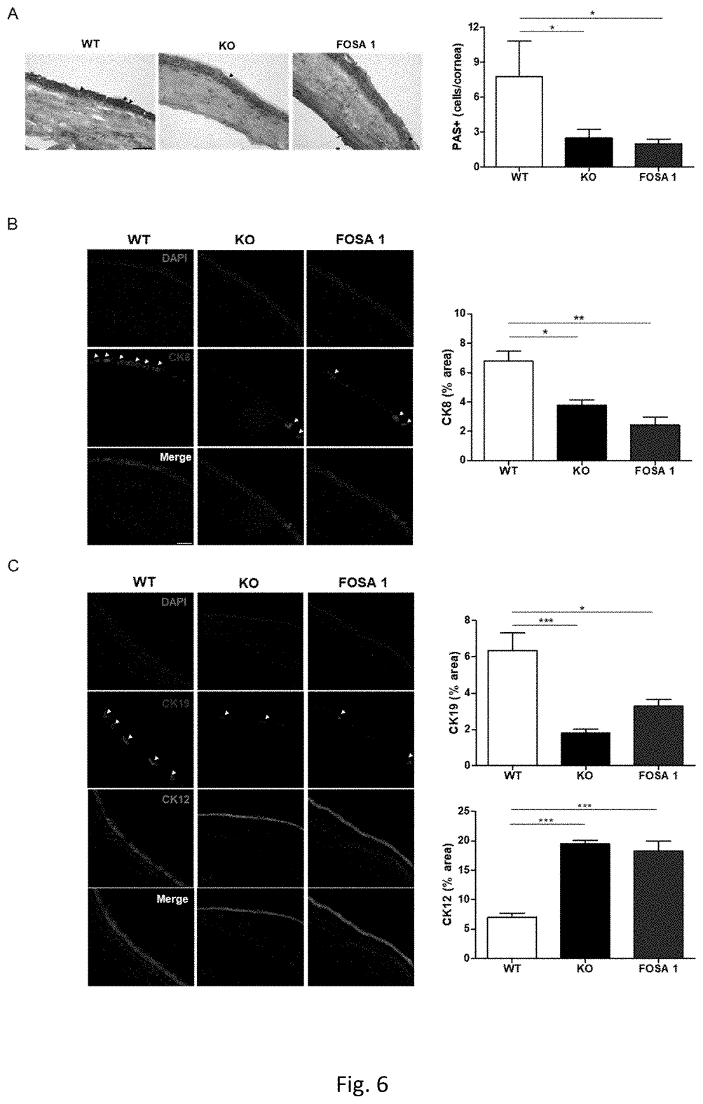

FIG. 6. Substance P ablation or Neurokinin-1 receptor blockade reduces the number of goblet and conjunctival cells infiltrating the cornea. A—PAS staining for goblet cells identification in the cornea. Representative images are shown (40×). Scale bar=50 um. Reduced number of PAS+ cells is observed in TAC1-KO and FOSA 1 mice compared to WT. B—Cytokeratin 8 (CK8) staining for conjunctival cells identification. Representative images of the immunofluorescence are shown (20×). Scale bar=75 um. Reduced number of CK8⁺ conjunctival cells is observed in TAC1-KO and FOSA 1 mice compared to WT. C-Cytokeratin 12 (CK12) and Cytokeratin 19 (CK19) staining for corneal and conjunctival cells, respectively. Representative images are shown (20×). Scale bar=75 um. When compared to WT, TAC1-KO, and FOSA 1 mice exhibited an increase in CK12 expression in the corneal epithelium. In addition, TAC-KO and FOSA 1 mice showed a significant decrease in CK19 staining. Scale bar=75 um. Graphs show mean values ±SEM; statistical analysis by One-way ANOVA followed by Tukey's test. *p<0.05 **p<0.01 ***p<0.001

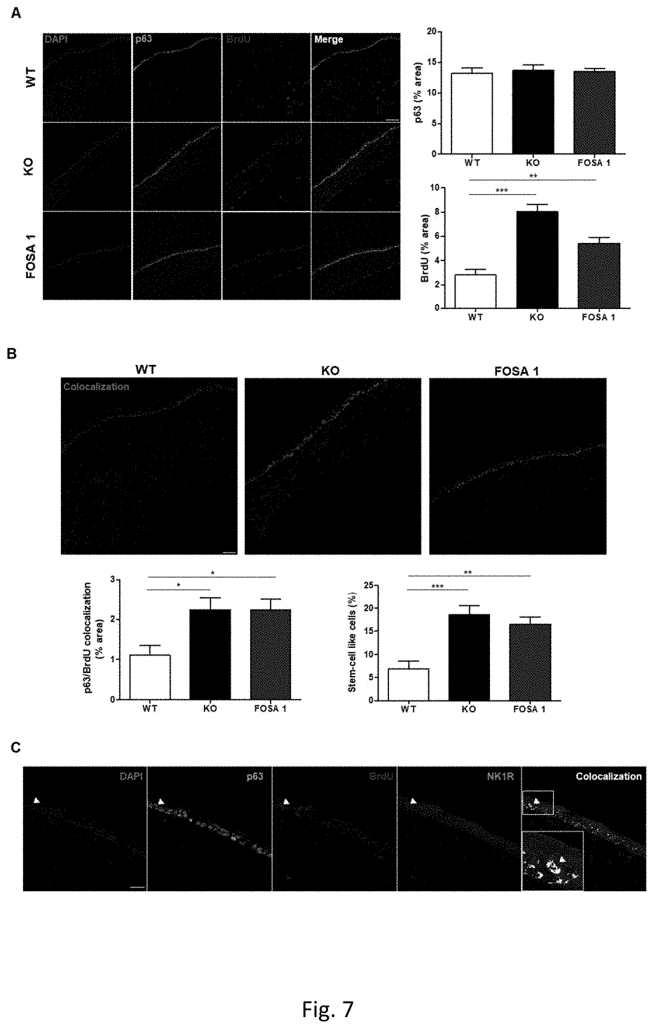

FIG. 7. Substance P ablation or Neurokinin-1 receptor blockade alleviate stem cell deficiency. A—p63 and BrdU staining for stem cell identification. Representative pictures are shown (20×). Scale bar=75 um. In comparison to WT mice, TAC1-KO and FOSA 1 mice showed a significant increase in BrdU positivity in the corneal epithelium, while no difference were found in p63 expression. B—Colocalization between p63 and BrdU was performed. Representative colocalization points are shown (20×). Scale bar=75 um. TAC1-KO and FOSA 1 mice exhibited an increased percentage of colocalization between p63 and BrdU. Stem cell-like cells were identified when the colocalized points represented more than 50% of the nucleus, using DAPI as a reference. An increase number of these cells was found in TAC1-KO and FOSA 1 groups. C—Neurokinin-1 receptor, BrdU, and p63 triple staining. Representative pictures are shown (40×). Scale bar=25 um. As it is shown, NK1R is expressed by both corneal epithelial and stem cell-like cells. Graphs show mean values ±SEM; statistical analysis by One-way ANOVA followed by Tukey's test. *p<0.05 **p<0.01 ***p<0.001

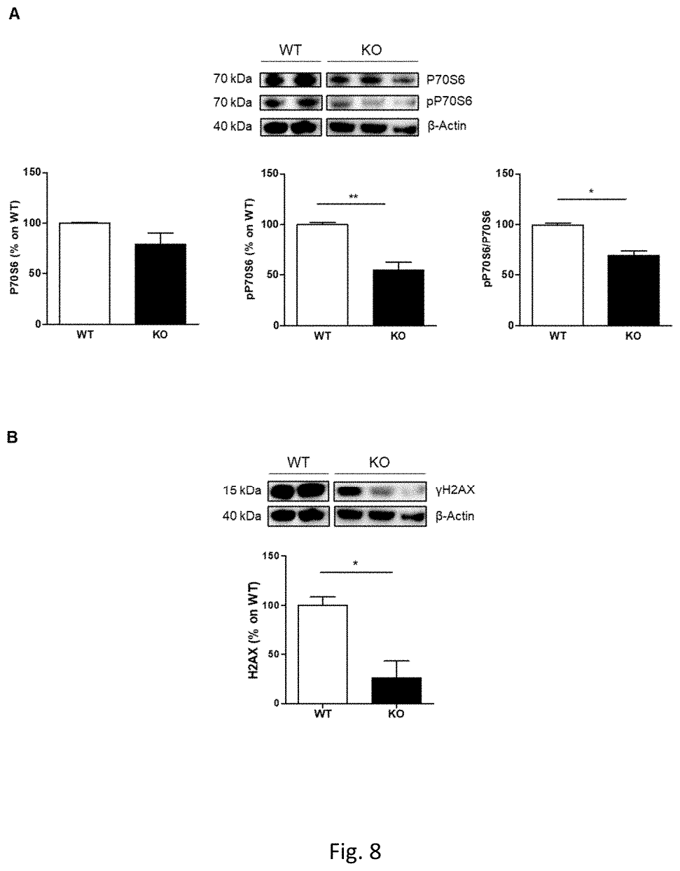

FIG. 8. Substance P ablation results in decreased mTOR signaling and senescence in corneal epithelium. A—Expression of P70S6 and pP70S6, downstream proteins in mTOR pathway, evaluated by western blot. A representative blot is shown. When compared to WT, TAC1-KO mice showed a significant decrease in pP70S6 expression. A ratio between pP70S6 and P70S6 expression was performed in order to determine mTOR pathway activation. A decrease in this ratio was observed in TAC1-KO mice, compared to WT. B—Expression of H2AX as a senescence marker. A representative blot is shown. TAC1-KO mice exhibited a decreased H2AX expression in comparison to WT. Graphs show mean values ±SEM; statistical analysis by unpaired Student's t-test *p<0.05

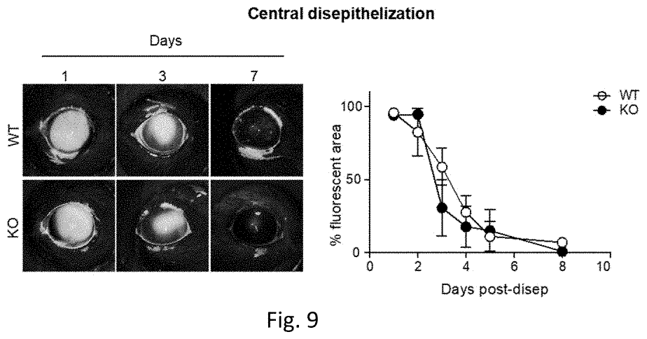

FIG. 9: Substance P ablation does not improve the wound closure rate when the limbus is intact. A—Wound healing progress evaluated by fluorescein assay. No difference were Graphs show mean values ±SEM; statistical analysis by two-way ANOVA.

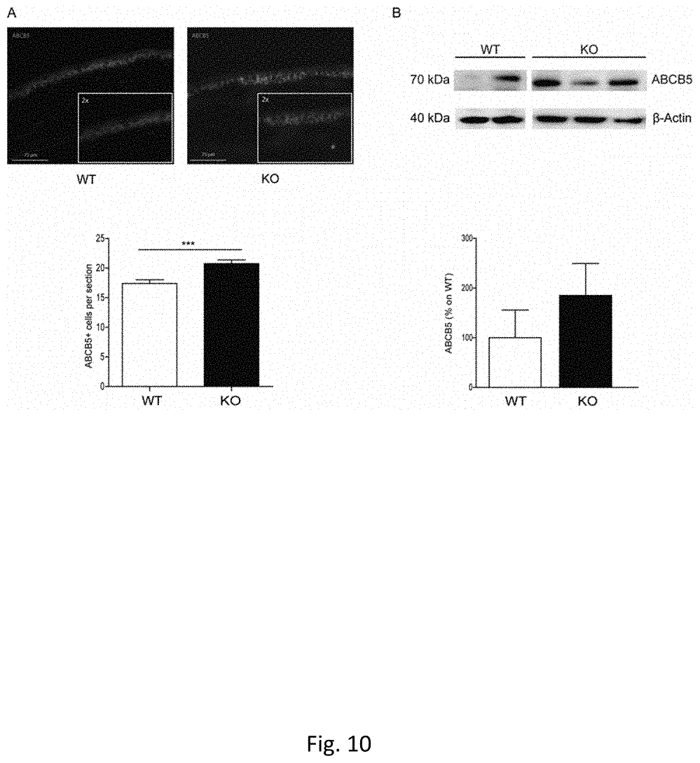

FIG. 10: A—ABCB5 expression as a stem cell marker evaluated by immunofluorescence. Representative images are shown (20×). When compared to WT, TAC1-KO mice exhibit an increase in ABCB5 expression in corneal epithelium. B—ABCB5 expression assessed by western blot. Graphs show mean values ±SEM; statistical analysis by unpaired Student's t-test *p<0.05

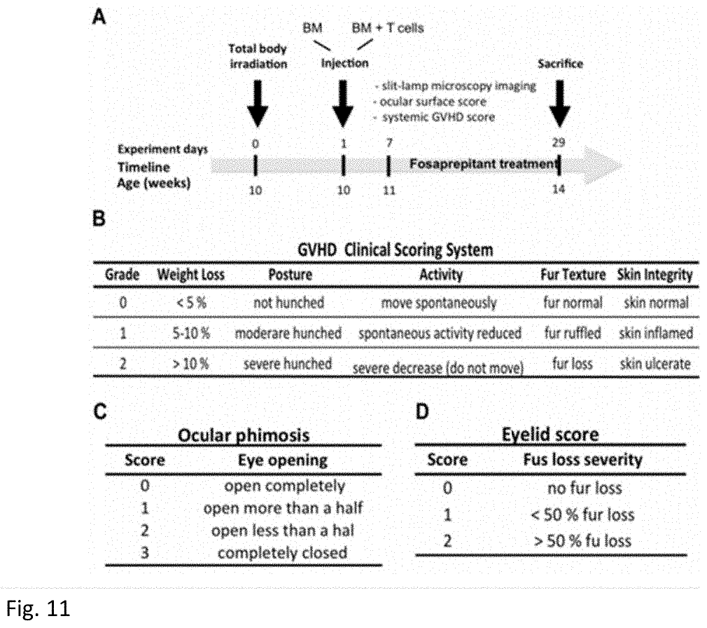

FIG. 11: Experimental design. (A) Schematic representation of the clinical model and treatment. (B) GVHD (graft versus host disease) Clinical scoring system. (C) Ocular phimosis score. (D) Eye-lid score.

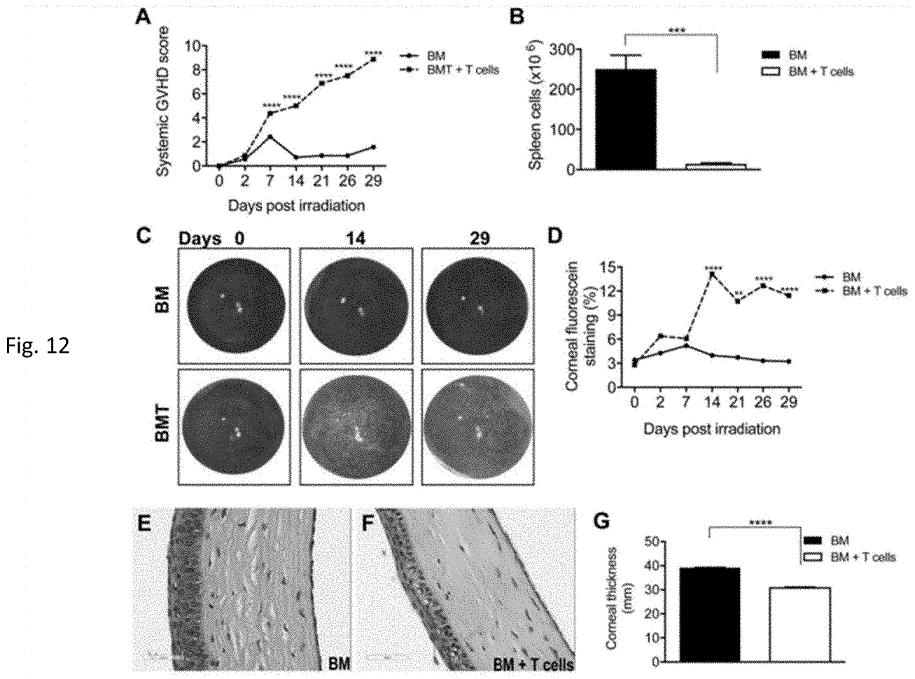

FIG. 12: Acute GVHD induces epithelial corneal damage. (A) Inventors first measured the systemic GVHD scores in this preclinical mouse model. Mice that received T cells (BM+T) developed high systemic GHVD scoring compared with mice that only received bone marrow (BM). (B) Quantification of spleen cells. The significant reduction observed in the BM+T cells group confirms the onset of GVHD (C) Corneas were examined and photographed under a slit-lamp microscope. Representative fluorescein pictures from BM and BM+T groups at day 0, 14 and 29 are shown. (D) Quantification of fluorescein staining showed an increase in BM+T group after 14 days. Graphs represent mean values ±SEM; Statistical analysis by Unpaired Student's t test or One-way ANOVA, following Bonferroni post hoc test, when it corresponds. **p<0.01, *** p<0.001, ****p<0.0001.

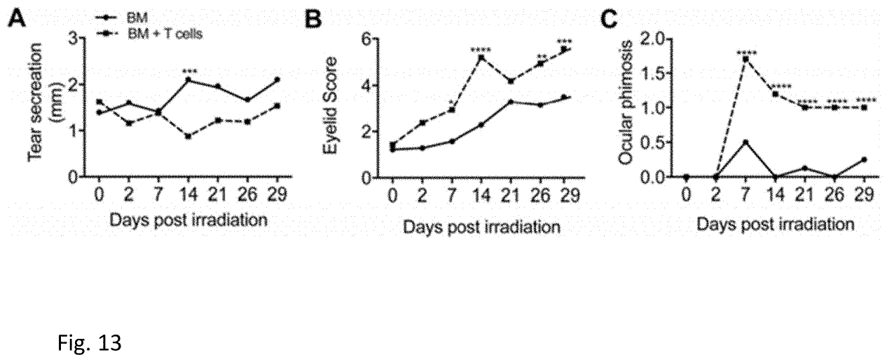

FIG. 13: Clinical ocular changes in acute GVHD mouse model. (A) Tear secretion assessment by phenol red in BM and BM+T mice. Significant differences were observed on day 14 between both groups. (B) Eye-lid score assessment for BM and BM+T groups. Significant differences were observed between groups since day 7. (C) Phimosis score. Acute GVHD significant increase the onset of phimosis in BM+T. Graphs represent mean values ±SEM; Statistical analysis by One-way ANOVA, following Bonferroni post hoc test. **p<0.01, ***p<0.001,****p <0.0001.

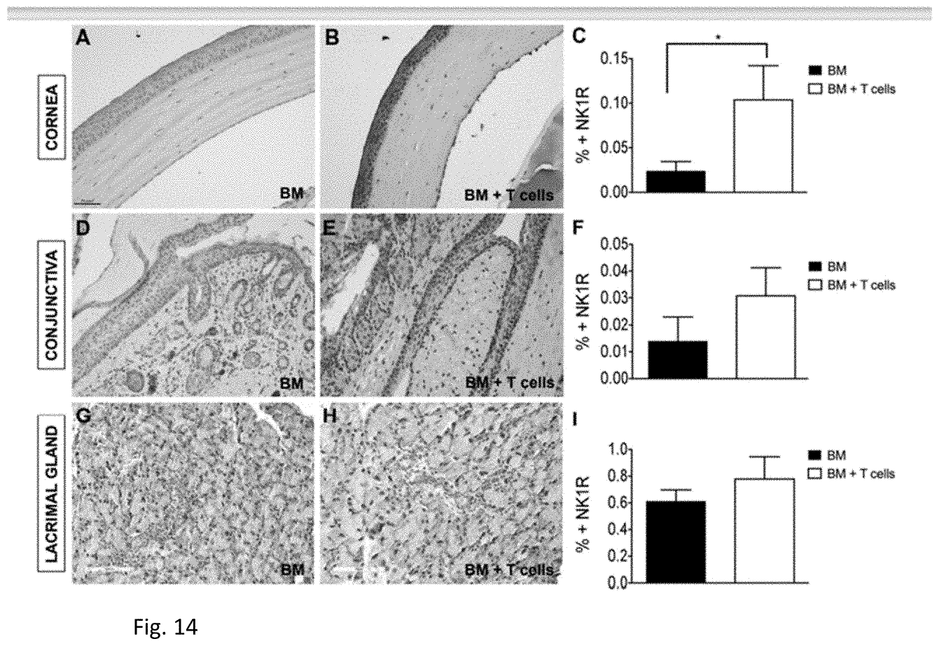

FIG. 14: Acute GVHD induces the expression of Neurokinin-1 receptor in the cornea. NK1R expression was assessed by immunohistochemistry at the end of the experiment (day 29). Representative images (20×) of NK1R staining are shown. (A-C) BM+T mice showed a significant increase of NK1R expression in the cornea. (D-F) Conjunctival expression of NK1R. No significant differences were found. (G-I) NK1R expression in the lacrimal gland. No significant differences were found. Graphs represent mean values ±SEM; Statistical analysis by Unpaired Student's t test. *p<0.05.

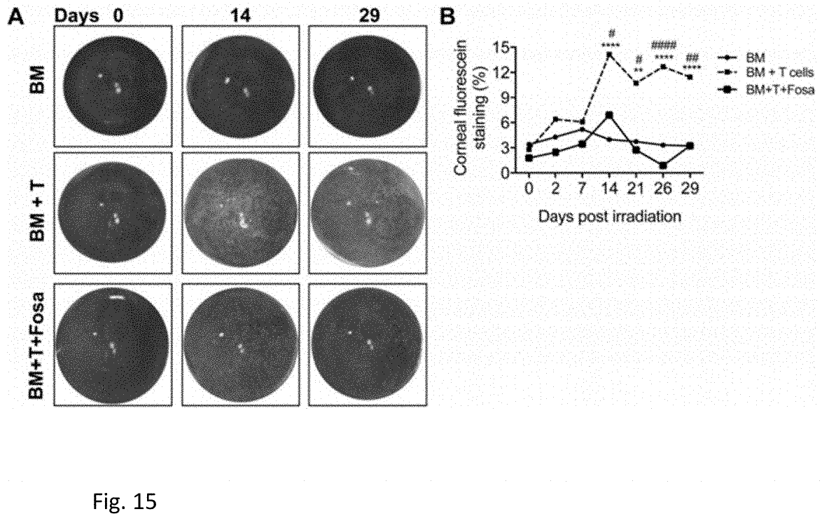

FIG. 15: Topical Fosaprepitant treatment reduces epithelial corneal damage. (A) Corneas were examined and photographed under a slit-lamp microscope. Representative fluorescein pictures from BM, BM+T, BM+T+Fosa at day 0, 14, and 29 are shown. A reduction in fluorescein staining was observed in the BM+T+Fosa group when compared to BM+T mice. Graphs represent mean values ±SEM; Statistical analysis by One-way ANOVA, following Bonferroni post hoc test. **p<0.01, ***p<0.001,****p<0.0001 vs BM. #p<0.05, ##p<0.01, ####p<0.0001 vs BM+T+Fosa.

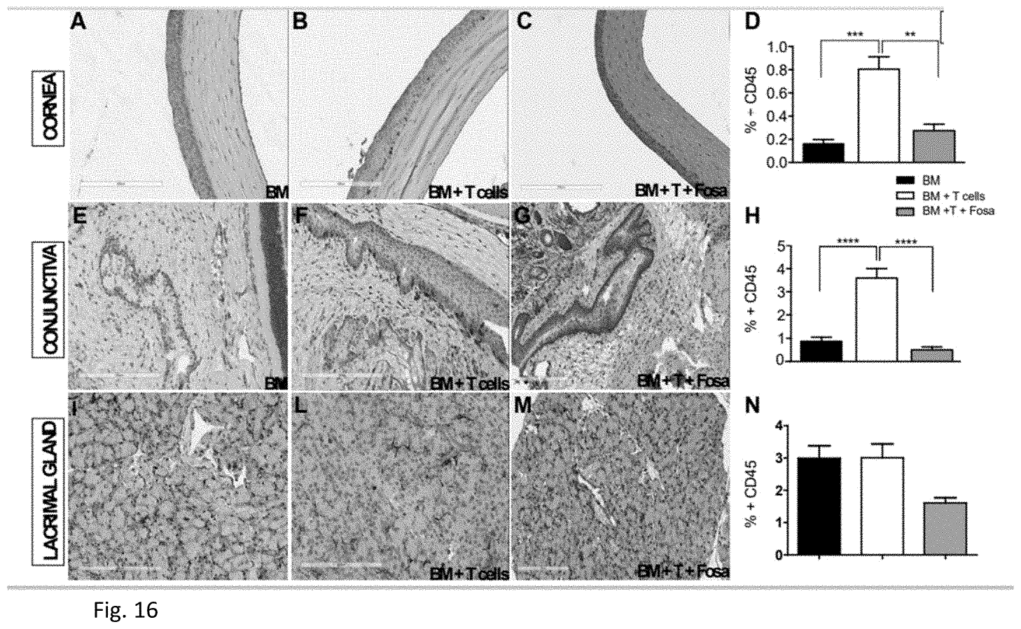

FIG. 16: Inflammatory cell infiltration is reduced by topical Fosaprepitant treatment. CD45+ cells were assessed by immunohistochemistry at the end of the experiment (day 29). (A-D) Representative images of CD45 staining in the cornea (20×). BM+T mice showed a significant increase in CD45+ cells, that was reduced by Fosaprepitant treatment. (E-H) Representative images of CD45+ staining in the conjunctiva (20×). BM+T mice showed a significant increase in CD45+ cells, that was reduced by Fosaprepitant treatment. (I-N) Representative images of CD45+ staining in the lacrimal gland (20×). No significant differences were found among groups. Graphs represent mean values ±SEM; Statistical analysis by One-way ANOVA, following Bonferroni post hoc test. **p<0.01, *** p<0.001, ****p<0.0001.

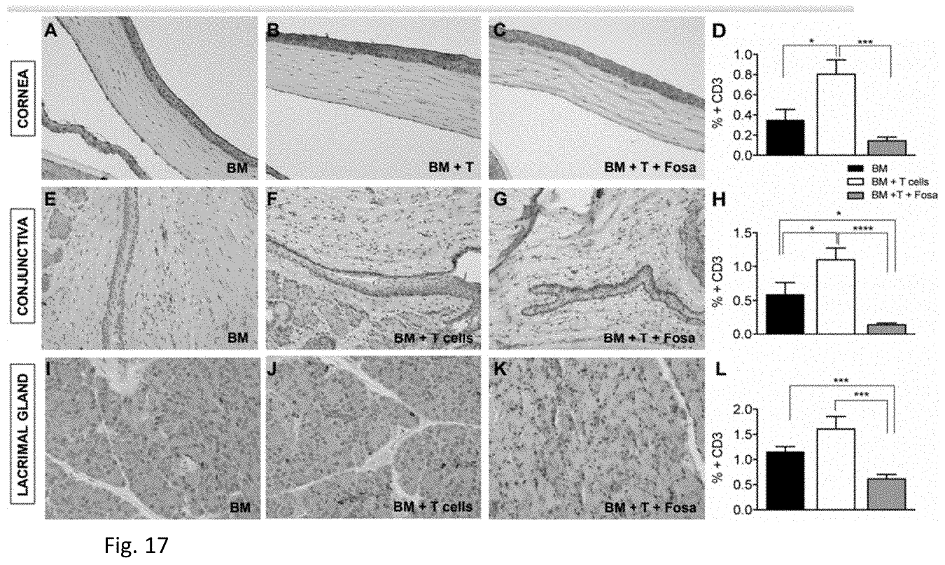

FIG. 17: Fosaprepitant treatment reduces CD3 cell infiltration. CD3+ cells were assessed by immunohistochemistry at the end of the experiment (day 29). (A-D) Representative images of CD3 staining in the cornea (20×). BM+T mice showed a significant increase in CD3+ cells, that was reduced by Fosaprepitant treatment. (E-H) Representative images of CD3 staining in the conjunctiva (20×). BM+T mice showed a significant increase in CD3+ cells, that was reduced by Fosaprepitant treatment. (I-L) Representative images of CD3+ staining in the lacrimal gland (20×). Fosaprepitant treatment reduced the CD3+ cell infiltration. Graphs represent mean values ±SEM; Statistical analysis by One-way ANOVA, following Bonferroni post hoc test. **p<0.01, *** p<0.001, ****p<0.0001.

### DETAILED DESCRIPTION OF THE INVENTION

### Examples

### Example 1

Materials and Methods

Patients

Tear samples were collected from 5 eyes of 4 patients (mean age, 47 years; range, 29-68 years;

2 females and 2 males) affected by stem cell deficiency. A table summarizing patient demographics is shown in FIG. 1-A. Slit-lamp pictures were taken after tear collection, and stem cell deficiency was clinically classified using the clinical scoring system previously described [18]. Control tear samples were obtained from age-matched healthy volunteers presenting no ocular surface diseases.

An informed written consent was obtained from all patients, prior to enrollment at the Cornea Unit of the San Raffaele Hospital, Milan. The study was approved by the Institutional Review Board of the San Raffaele Institute and followed the Tenets of the Declaration of Helsinki.

Tear Collection

Tears were collected using a single polyurethane mini-sponge (PeleTim; VOCO GmbH, Cuxhaven, Germany) placed over the lids margin at the junction of the lateral and middle thirds of the lower eyelids for 1 minute, without anesthesia, as previously described [19]. The sponge was recovered, avoiding the tear reflex as much as possible, placed in a truncated micropipette tip adapted to a sterile 1.5 mL tube, and centrifuged at 3.5 g for 5 minutes. Tear samples were immediately stored at −80° C. until further analysis. Samples were obtained before any clinical tests to avoid any interference.

Evaluation of Substance P Levels in Human Tears

Levels of SP in human tear samples were measured using a multiplex bead-based assay, Milliplex Human Neuropeptide Magnetic Panel (HNPMAG-35K; Millipore, Burlington, Mass., USA). Samples were diluted (1:3) for the analysis, and tested in duplicate. Measurements were performed using a Luminex 100 Bioanalyzer (Luminex Technologies, Austin, Tex., USA).

Disepithelization Model

Eight-week-old C57BL6/N (Charles River, Calco, Italy) (WT mice, n=8) and B6.Cg-Tac1tm1Bbm/J (Jackson Laboratories, Bar Harbor, Me., USA) (TAC1-KO mice, n=8) male mice were used in all experiments. Each animal was anesthetized with intraperitoneal injection of tribromoethanol (250 mg/kg) and a topical anesthetic was applied to their ocular surface prior to the surgical procedure. Disepithelization was performed based on the model previously described [20], using a blunt spatula to scrap the entire corneal epithelium from limbus to limbus. After wounding, eyes were treated with ophthalmic moxifloxacin 1 time/day for 1 week to minimize inflammation. Fourteen days after disepithelization mice were euthanized using carbon dioxide inhalation and subsequent cervical dislocation. Corneas were collected for immunostaining and western blot analysis. All experimental protocols were approved by the Animal Care and Use Committee of the Istituto di Ricovero e cura a Carattere Scientifico San Raffaele Scientific Institute, in accordance with the ARVO Statement for the Use of Animals in Ophthalmic and Vision Research.

Fosaprepitant Treatment

To evaluate the pharmacological inhibition of Substance P, an additional group of mice (WT-FOSA, n=6) received Fosaprepitant 10 mg/mL (IVEMEND, Merck Sharp & Dohme Ltd., Hoddes-don, UK) dissolved in a total volume of 10 μL phosphate-buffered saline (PBS, Sigma-Aldrich, St. Louis, Mo., USA) or 10 μL of vehicle as control, topically four times a day in both eyes for 14 days. The treatments started 1 day after corneal dispepithelization.

Corneal Opacity and Wound Healing

Every day after disepithelization, in vivo corneal photographs were taken using a digital camera (EOS 30D; Canon, Tokyo, Japan) attached to a slit-lamp microscope (Photoslitmap 40 SL-P; Zeiss, Oberkochen, Germany) to asses corneal condition. Corneal opacity was evaluated using a grading score (from 0-4; 0=completely clear, 4=completely opaque) previously described [21]. Additionally, in vivo corneal fluorescein staining was used to evaluate wound healing. Eyes were photographed under a blue light (with or without a yellow filter) of the slit-lamp microscope SL 990. The percentage of green fluorescent area on the total corneal area was evaluated analyzing images by Image J software (National Institutes of Health, Bethesda, Md., USA).

Goblet Cell Identification

Periodic acid Schiff (PAS) staining was performed to identify the presence of goblet cells. Briefly, eyes were removed and fixed with 4% paraformaldehyde (PFA) (Sigma-Aldrich) for 10 min at RT. After gradient dehydration in 10%, 20%, and 30% sucrose in PBS for 2 h each, the samples were embedded in optimal cutting temperature medium (Killik, Bio-Optica, Milan) and sectioned at 8 μm. Then, the following steps were performed in sequence: section immersion in PAS solution for 10 min, wash in distilled water, immersion in Schiff reagent for 20 min, wash in distilled water, immersion in potassium methabisulphite solution for 2 min and then in fixative solution for 2 min, wash in distilled water, immersion in Mayer's Hemalum for 3 min, wash in running tap water for 5 min. Dehydration through ascending alcohols, clear in xylene and mounting. The number of PAS+ cells were quantified using Image J software (National Institutes of Health, Bethesda, Md., USA).

Conjunctival Epithelial Cell Identification

The presence of conjunctival epithelial cells was evaluated by immunofluorescence. Briefly, eyes were removed and fixed with 4% paraformaldehyde (PFA) (Sigma-Aldrich) for 10 min at RT. After gradient dehydration in 10%, 20%, and 30% sucrose in PBS for 2 h each, the samples were embedded in optimal cutting temperature medium (Killik, Bio-Optica, Milan) and sectioned at 8 pm. Cryosections were blocked in 2% BSA, 0.3% Triton X-100 (Sigma-Aldrich), and 0.05% Tween-20 in PBS for 1 h at RT and incubated overnight at 4° C. with rabbit anti-keratin-8 (Krt8) (AB59400, Abcam), followed by AlexaFluor 594 donkey anti-rabbit IgG (A21207, Life Technologies) for 1 h at RT, diluted in 2% BSA, 0.3% Triton X-100, and 0.05% Tween-20. Samples were counterstained with 40,6-diamidino-2-phenylindole (DAPI, Vector Laboratories, Inc., Burlingame, Calif., USA) mounted, and photographed by epifluorescence microscope (Leica CTR5500; Leica Microsystems, Wetzlar, Germany). The number of Krt8+ cells were quantified using Image J software (National Institutes of Health, Bethesda, Md., USA).

Evaluation of ATP-Binding Cassette Sub-Family B Member 5 (ABCB5) Expression

The expression of ABCB5 was assayed by immunofluorescence and Western Blot. For immunofluorescence, sample preparation as well as immunostaining were previously described in section 2.8. The primary antibody used was a rabbit anti-ABCB5 (NBP1-77687, Novus Biological), followed by AlexaFluor 488 donkey anti rabbit IgG, diluted in 2% BSA, 0.3% Triton X-100, and 0.05% Tween-20. The number of ABCB5⁺ cells were quantified using Image J software (National Institutes of Health, Bethesda, Md., USA).

For Western Blot analysis, corneal epithelium was isolated after 30 minutes of EDTA treatment (Sigma-Aldrich Corp., St. Louis, Mo., USA) at 37° C. A pool of four corneal epithelia from different animals were used. A sample of 5 μg of proteins from WT or TAC1-KO epithelia was re-suspended in NuPAGE LDS reducing Sample Buffer (Thermo Fisher), resolved on NuPAGE 4-12% Bis-Tris Protein Gels (Thermo Fisher) and electro-transferred to nitrocellulose membranes (Amersham, Little Chalfont, UK). Red Ponceau S staining (Sigma-Aldrich) was used to evaluate protein transfer. Membranes were blocked in a Tris buffered solution (TBS) 5% milk, 0.1% Tween 20 and incubated overnight with rabbit anti-ABCB5 (AP6122a-ev, Abcepta) at 4° C. under gentle shaking. Subsequently, membranes were incubated at RT for 1 h with HRP-conjugated secondary antibodies (NA9340V, Ge Healthcare) followed by chemiluminescence reaction performed with ECL detection reagent (Ge Healthcare) and film exposure. Expression of β-actin HRP conjugated mouse monoclonal antibody (A3854, Merck) was used as loading control. Bands were quantified using Image Lab™ software (Version 6.0.1, Bio-Rad Laboratories, Hercules, Calif., USA).

Expression of Proteins Related to the Target of Rapamycin (mTOR) Signaling Pathway

The expression of p70 S6 Kinase (P70S6), phospho-p70 S6 Kinase (pP70S6), and phospho-histone 2-AX (γH2AX) was evaluated by Western Blot, as previously described in section 2.9. The following primary antibodies were used: rabbit anti-P70S6 (9202, Cell Signaling), mouse anti-pP70S6 (9206, Cell Signaling), and rabbit anti γH2AX (NB100-384, Novus Biologicals), followed by anti-mouse or anti rabbit-HRP-conjugated secondary antibodies (NA9310V and NA9340V respectively, Ge Healthcare). As control loading, expression of β-actin HRP-conjugated mouse monoclonal antibody (A3854, Merck) was used. Bands were quantified using Image J software (National Institutes of Health, Bethesda, Md., USA).

Statistical Analysis

Statistical calculation was performed using GraphPad Prism 5.0 software (GraphPad Software, La Jolla, Calif., USA). Data were expressed as mean±standard error of mean (SEM). The statistical significance of the differences between the two groups was calculated by unpaired Student's t-test, α=0.05. For corneal wound closure and corneal opacity analysis, two-way ANOVA was performed, considering p<0.05 statistically significant.

Results

Substance P Levels are Increased in the Tears of Patients Affected with Stem Cell Deficiency.

Characteristics, diagnosis and SCD severity of all the patients participating in this study are displayed in FIG. 1-A. In FIG. 1-B, representative images of slit-lamp and fluorescein of patient no 2 right eye are displayed. As it is observed, there is a great extent of limbus and corneal involvement, consisted with the severity of the disease. As it is shown in FIG. 1-C, tear levels of SP were found increased in SCD-affected patients when compare to age-matched healthy volunteers (42%, p<0.05).

Substance P Ablation Improves Corneal Epithelial Wound Healing and Corneal Transparency

After disepithelization, in vivo corneal photographs were taken every day in order to evaluate wound healing progress and corneal opacity (FIG. 2). When compared to WT, TAC1-KO mice showed significant faster epithelial wound healing, starting from the third day after disepithelization. By day 14, wound healing was 57% higher in TAC1-KO mice (p<0.0001), as it is shown by the reduced fluorescein staining (FIG. 2, A). Additionally, TAC1-KO mice presented a significant increase in corneal transparency compared to WT mice (FIG. 2, B). By day 14, corneal transparency was 70% higher in TAC1-KO mice than in WT (p<0.001).

Substance P Ablation Reduces the Number of Goblet and Conjunctival Cells Infiltrating the Cornea, and Alleviates Stem Cell Deficiency

PAS staining was employed in order to identify goblet cells in the cornea [22]. Representative images (40×) are shown in FIG. 3-A. After 14 days of disepithelilzation, TAC1-KO mice corneas presented a decrease of PAS+ cells when compared to WT (69%, p<0.05).

To evaluate conjunctival cells infiltrating the cornea, Krt8 staining was performed. Representative images (20×) of the immunofluorescence are shown in FIG. 3-B. After 14 days, TAC1-KO mice showed reduced number of Krt8⁺ conjunctival cells compared to WT (67%, p<0.05).

The ABCB5 expression was assayed as a stem cell marker. FIG. 3-C showed representative images (20×) of immunostaining and its quantification. When compared to WT, TAC1-KO mice exhibited an increase in ABCB5 expression in corneal epithelium (19%, p<0.05). FIG. 3-D showed ABCB5 expression evaluated by Western Blot. Bands of approximately 70 kDa and 40 kDa correspond to ABCB5 and β-actin, respectively. Compared to WT, TAC1-KO mice presented an increasing trend in ABCB5 expression, although not significant.

Substance P Ablation and its Pharmacological Inhibition Result in Decreased mTOR Signaling and Senescence in Corneal Epithelium

The expression of P70S6 and pP70S6, two downstream proteins in mTOR pathway, was evaluated by Western Blot. A representative blot is shown in FIG. 4-A. Bands of approximately 70 kDa, 70 kDa, and 40 kDa correspond to pP70S6, P70S6, and β-actin, respectively. When compared to WT, TAC1-KO and WT-FOSA mice showed a significant decrease in pP70S6 expression (51% and 38%, respectively, p<0.05). A ratio between pP70S6 and P70S6 expression was performed in order to determine mTOR pathway activation. A decrease in this ratio was observed in both TAC1-KO and WT-FOSA mice, compared to WT. No significant differences were found between WT-FOSA and TAC1-KO in P70S6 or pP70S6 expression.

Finally, γH2AX expression was assayed as a senescence marker and as a target downregulated by mTOR pathway. A representative blot is shown in FIG. 4-B. Bands of approximately 15 kDa and 40 kDa correspond to γH2AX and β-actin, respectively. TAC1-KO and WT-FOSA mice displayed a decreased γH2AX expression in comparison to WT (53% and 26%, p<0.05). No significant differences were found between WT-FOSA and TAC1-KO in γH2AX expression. Epithelial stem cells are vital to repopulate the cornea after injury and prevent vision loss [23-25]. Many ocular diseases, such as corneal burns, severe dry eye and autoimmune disorders can induce SCD [14,26,27]. Here, the inventors report the effect of SP ablation, or its pharmacological inhibition, on the wound healing efficiency following epithelial stem cell injury. The inventors show that overexpression of SP is associated with SCD in human subjects. The inventors also found that SP expression aggravates SCD and impairs the expression of epithelial stem cell marker ABCB5 in a well-established mouse model [20,28,29]. SP-mediated activation of neurokinin 1 receptor aggravates corneal SCD through stimulation of the mTOR pathway eventually leading to accelerated corneal epithelial cell senescence. Finally, the inventors demonstrate that topical application of neurokinin 1 receptor antagonist Fosaprepitant ameliorates corneal SCD.

It is established that chronic inflammation can induce depletion of stem cells in other body sites as well as other cellular mechanism such as DNA damage and mitochondrial dysfunction [30,31]. Interestingly, severe and/or chronic inflammation of the ocular surface is also a well-known cause of SCD in humans [24], which is the reason of the common prescription of anti-inflammatory medication in SCD-affected patients. However, up to date there is no evidence that this medication could increase the stem cell pool in the cornea. Recalcitrant inflammation is also the most common cause associated with failure of autologous corneal epithelial stem cell transplantation [32]. Substance P is a neuropeptide with well-known immunomodulatory activities. In this vein, the key role of SP and its neurokinin 1 receptor activation in promoting leukocyte infiltration, neovascularization, and scarring in the ocular surface has been reported before [8,9,33]. The present result that patients affected with SCD express higher levels of SP in tears corroborate these previous findings. It is interesting to remark that the one patient affected with bilateral SCD showed higher SP levels in the eye with more severe SCD.

The severe reduction of epithelial stem cells results in defective wound healing, leading to neovascularization, chronic inflammation, corneal opacity, and vision loss [23,32]. Since SP was increased in SCD-affected patients, the inventors decided to use an experimental model of stem cell deficiency in order to investigate whether SP ablation could improve corneal wound healing when stem cell loss takes place. Interestingly, the inventors found that after the wound the clinical outcomes associated with SCD such as non-healing epithelial defects and conjunctival migration onto the cornea were ameliorated by SP ablation. Moreover, the inventors detected that the stem cell pool was increased, alleviating SCD. It should be noted that previous works suggest that SP could improve wound healing [34-36], although the specific effect of SP on epithelial stem cell markers was not studied.

The target of rapamycin (mTOR) signaling has been pointed out as one of cellular pathway controlling aging, cellular senescence, and lifespan by regulation of stem cell proliferation and differentiation in oral and intestinal mucosa, and the skin [38-40]. The activity of mTOR appears to be time-dependent, since it has been shown that long-term activation of mTOR leads to stem cell exhaustion and its inhibition preserves adult stem cell function [41]. DNA damage is one of the major causes of stem cell exhaustion, inhibiting self-renewal and inducing senescence in several tissues [42-44]. The phosphorylation of H2AX, a widely-used senescence marker, is an early event that follows genotoxic stress and plays an essential role in the recruitment of DNA repair proteins to the site of damage [45]. Interestingly, mTOR restraining has been associated with the suppression of cellular senescence in several tissues [38,40,46], and specifically prevent H2AX phosphorylation [47]. In this sense, the inventors found a similar mechanism on the corneal epithelium: in a highly-inflammatory environment such as a total disepithelization, the release of SP induces mTOR signaling by binding NK1R and promotes cellular senescence, supporting the hypothesis that persistent activation of mTOR pathway leads to stem cell exhaustion. In line with our results, it has been recently reported that mTOR inhibition with rapamycin prolongs the survival of corneal epithelial cell in vitro and maintains their proliferative potential [48]. Additionally, mTOR plays a key role in regulating scarring, neovascularization, and inflammation in the cornea [49,50]. On the contrary, a recent study conducted by Park et al. showed that rapamycin aggravates corneal epithelial stem cell deficiency by upregulating the inflammatory response. It is well-known that rapamycin almost completely inhibit mTOR activity by blocking substrate recruitment [52]. Herein, the inventors showed that SP ablation or pharmacological inhibition of NK1R promote a partial inhibition of mTOR activity (almost 50%), introducing the novel concept that a discrete function of mTOR on the corneal epithelium is beneficial to alleviate SCD, by increasing the pool of epithelial stem cells and preventing cell senescence.

Nowadays, SCD is a major clinical problem and an area of unmet medical need. While cellular therapies are now available, they cannot be accessed by everyone. Altogether present data support that SP can favor SCD through mTOR signaling, promoting cell senescence. Therefore, inhibition of SP activity by means of topical neurokinin 1 receptor antagonists represents an attractive option to treat stem cell deficiency, in particular corneal epithelial stem cell deficiency. The present data suggest that hyper-expression of SP can induce senescence and exhaustion of residual stem cells through activation of neurokinin 1 receptor. These results have relevant translational implications, in particular because neurokinin 1 receptor antagonists can be formulated for ocular use.

### Example 2

Materials and Methods

Stem Cell Deficiency Model

Eight-week-old male mice C57BL6/N (Charles River, Calco, Italy) (WT mice, n=18) and Tac1-deficient B6.Cg-Tac1tm1Bbm/J (Jackson Laboratories, Bar Harbor, Me., USA) (TAC1-KO mice, n=18) were used in all experiments. TAC1-KO mice are homozygous null for the Tac1 gene. They are viable, fertile, normal in size and do not display any gross physical or behavioral abnormalities. In previous work, inventors showed that TAC1-KO mice did not present ocular abnormalities. The corneal thickness and transparency as well as the corneal nerve length are similar to C57BL/6 mice [Barbariga M, Rabiolo A, Fonteyne P, Bignami F, Rama P, Ferrari G. The effect of aging on nerve morphology and substance P expression in mouse and human corneas. Investig Ophthalmol Vis Sci 2018; 59:5329-35. https://doi.org/10.1167/iovs.18-24707.]. Each animal was anesthetized with an intraperitoneal injection of tribromoethanol (250 mg/kg) and a topical anesthetic was applied to their ocular surface prior to the surgical procedure. Total disepithelization was performed based on the model previously described [Amirjamshidi H, Milani B Y, Sagha H M, Movahedan A, Shafiq M A, Lavker R M, et al. Limbal fibroblast conditioned media: A non-invasive treatment for limbal stem cell deficiency. Mol Vis 2011; 17:658], using a blunt spatula to scrap the entire corneal epithelium, including the limbus. Then, fluorescein staining was used to confirm complete removal of the corneal epithelium under the blue light of a slit-lamp microscope. After wounding, eyes were treated with ophthalmic moxifloxacin 1 time/day for 1 week to minimize inflammation. Fourteen days after disepithelization mice were euthanized using carbon dioxide inhalation and subsequent cervical dislocation. Corneas were collected for immunostaining and western blot analysis. All experimental protocols were approved by the Animal Care and Use Committee of the Istituto di Ricovero e cura a Carattere Scientifico San Raffaele Scientific Institute, in accordance with the ARVO Statement for the Use of Animals in Ophthalmic and Vision Research.

Fosaprepitant Treatment

To evaluate the pharmacological inhibition of Substance P, mice received 10 μL of Fosaprepitant topically (IVEMEND, Merck Sharp & Dohme Ltd., Hoddes-don, UK) in two different concentrations: 0.1 mg/mL (FOSA 0.1, n=5) or 1 mg/mL (FOSA 1, n=5) 4 times/day every 2 hours for 16 days. Control animals received instead 10 μL of vehicle (PBS) (WT, n=5). The treatments started the same day that corneal disepithelization was performed.

Corneal Opacity and Wound Healing

Every day after disepithelization, in vivo corneal photographs were taken using a digital camera (EOS 30D; Canon, Tokyo, Japan) attached to a slit-lamp microscope (Photoslitmap 40 SL-P; Zeiss, Oberkochen, Germany) to assess corneal condition. Corneal opacity was evaluated using a grading score (from 0-4; 0=completely clear, 4=completely opaque) previously described [Yoeruek E, Deuter C, Gieselmann S, Saygili O, Spitzer M S, Tatar O, et al. Long-term visual acuity and its predictors after cataract surgery in patients with uveitis. Eur J Ophthalmol 2010; 20:694-701. https://doi.org/10.1177/112067211002000409]. Additionally, in vivo corneal fluorescein staining was used to evaluate wound closure. Eyes were photographed under blue light (with or without a yellow filter) of the slit-lamp microscope. The percentage of green fluorescent area on the total corneal area was evaluated by analyzing images by Image J software (National Institutes of Health, Bethesda, Md., USA).

Goblet Cell Identification

To identify the presence of goblet cells, periodic acid Schiff (PAS) staining was performed following the same experimental protocol previously described [Ferrari G, Bignami F, Giacomini C, Franchini S, Rama P. Safety and efficacy of topical infliximab in a mouse model of ocular surface scarring. Investig Ophthalmol Vis Sci 2013; 54:1680-8. https://doi.org/10.1167/iovs.12-10782]. The number of PAS+ cells was quantified using Image J software (National Institutes of Health, Bethesda, Md., USA), and the results were expressed as PAS+ cells per section.

Conjunctival and Corneal Epithelial Cell Identification

The presence of conjunctival epithelial cells was evaluated by immunofluorescence. Briefly, eyes were removed and carefully embedded in the same orientation in optimal cutting temperature medium (OCT Killik; Bio-Optica, Milan, Italy), and 8 pm cryosections were performed. After fixation in 4% paraformaldehyde (Sigma-Aldrich, San Louis, Mo., USA) for 20 min in ice, the sections were blocked with 2% bovine serum albumin, 0.1% Triton X-100 (Sigma-Aldrich), 10% normal donkey serum for 1 h at RT. The immunostaining was performed using rabbit anti-cytokeratin-8 (CK8) (1/200, AB59400, Abcam) or rat anti-cytokeratin-19 (CK19) (1/300, MABT913, Millipore) and guinea pig anti-cytokeratin-12 (CK12) (1/100, AP09545SU-N, Origene). Secondary detection was performed using AlexaFluor 594 donkey anti-rabbit IgG (1/1000, A21207, Life Technologies), AlexaFluor 594 donkey anti rat (1/1000, A21209, Life Technologies), AlexaFluor 488 donkey anti guinea pig (1/1000, A11073, Life Technologies), for 1 h at RT, diluted in blocking solution. Samples were counterstained with 4,6-diamidino-2-phenylindole (DAPI, Vector Laboratories, Inc., Burlingame, Calif., USA), mounted, and photographed by epifluorescence microscope (Leica CTR5500; Leica Microsystems, Wetzlar, Germany) (20×). The image analysis was performed using Image J software (National Institutes of Health, Bethesda, Md., USA). A negative control using only the secondary antibody was used to establish a fluorescence threshold. Then, the fluorescent intensity was quantified in terms of the percentage of positive area (% area) in the epithelium.

Stem Cell-Like Cells Detection

Immediately after corneal dispepithelization, animals were intraperitoneally injected with 5-bromo-2′-oxyuridine (BrdU) (50 ug/g body weight, ab142567, Abcam) twice a day (9 a.m and 4 p.m), for 7 days. At the indicated time points, eyes were processed for immunostaining as previously described in section 2.5. After a 10 min fixation with 4% paraformaldehyde on ice, samples were boiled in 10 mM citrate buffer (pH=6.00) in the microwave (700 W) for 4 min. After cool down to RT, samples were incubated with a blocking solution (2% BSA, 0.1 Triton X-100, 10% normal donkey serum in PBS) for 1 h at RT. Primary detection was performed with rabbit anti-p63 (1/300, ab124762, Abcam) for 1 h at RT, followed by AlexaFluor 488 donkey anti-rabbit (1/1000, A21206, Life Technologies) secondary antibody for 1 h at RT. A second fixation was performed with 4% paraformaldehyde during 20 min on ice and then transferred to 2 N HCl at 37° C. for 15 min followed by a neutralization step with borate buffer (pH=8.50) for 8 min. Samples were immersed again in blocking solution for 30 min and then incubated over night with rat anti-BrdU (1/100, ab6326, Abcam) primary antibody at 4° C., followed by AlexaFluor 594 donkey anti-rat (1/1000, A21209, Life Technologies) for 1 h at RT. Finally, sections were mounted with Vector Shield mounting medium (Vector Laboratories, Burlingame, Calif.), containing DAPI. The images were acquired by epifluorescence microscope (Leica CTR5500; Leica Microsystems, Wetzlar, Germany) (20×) and analyzed with Image J software. A negative control using only the secondary antibody was used to establish a fluorescence threshold. Then, the percentage of positive area (% area) for each marker was quantified in the epithelium. Colocalization between p63 and BrdU was assessed using the colocalization plug-in (National Institutes of Health, Bethesda, Md., USA), and then quantified as percentage of colocalized points. Then, stem cell-like cells were identified and quantified according to the score described by Pajoohesh-Ganji and co-workers [Pajoohesh-Ganji A, Pal-Ghosh S, Simmens S J, Stepp M A. Integrins in Slow-Cycling Corneal Epithelial Cells at the Limbus in the Mouse. Stem Cells 2006; 24:1075-86. https://doi.org/10.1634/stemcells.2005-0382]. A cell with more than 50% of the nucleus positive for both p63 and BrdU was considered a stem cell-like cell.

Neurokinin-1 Receptor (NK1R) Expression

To evaluate if the NK1R was expressed by epithelial stem cell-like cells, inventors performed a triple staining using p63 and BrdU as previously described in section 2.6. Primary goat anti-NKR1 (1/800, ab61705, Abcam) was co-incubated with p63 antibody for 1 h at RT, followed by AlexaFluor 633 donkey anti-goat (1/1000, A21082, Life Technologies) for 1 h at RT. Pictures were acquired in a DeltaVision™ Ultra microscope (GE healthcare, Chicago, Ill., USA) (40×) and image analysis was performed using Image J software (National Institutes of Health, Bethesda, Md., USA). A negative control using only the secondary antibody was used to establish a fluorescence threshold. Colocalization analysis was performed as described above. Expression of Proteins Related to the Target of Rapamycin (mTOR) Signaling Pathway

The expression of p70 S6 Kinase (P70S6), phospho-p70 S6 Kinase (pP70S6), and phospho-histone 2-AX (γH2AX) was evaluated by western blot. After corneal dissection, corneal epithelium was isolated after 30 minutes of EDTA treatment (Sigma-Aldrich Corp., St. Louis, Mo., USA) at 37° C. and re-suspended RIPA Buffer (Sigma-Aldrich) containing protease inhibitor. A pool of four corneal epithelia from different animals was used (WT, n=8; TAC1-KO, n=12). A sample of 10 μg of proteins from WT or TAC1-KO epithelia was resolved on NuPAGE 4-12% Bis-Tris Protein Gels (Thermo Fisher) and electro-transferred to nitrocellulose membranes (Amersham, Little Chalfont, UK). Red Ponceau staining (Sigma-Aldrich) was used to evaluate protein transfer. Membranes were blocked in a Tris-buffered solution (TBS) 3% BSA, 0.1% Tween 20 for 1 h at RT. The following primary antibodies were used: rabbit anti-P70S6 (1/1000, 9202, Cell Signaling), mouse anti-pP70S6 (1/1000, 9206, Cell Signaling), and rabbit anti-γH2AX (1/1000, NB100-384, Novus Biologicals), followed by anti-mouse or anti rabbit-HRP-conjugated secondary antibodies (1/5000, NA9310V and NA9340V respectively, Ge Healthcare). As loading control, expression of β-actin HRP-conjugated mouse monoclonal antibody (1/1000, A3854, Merck) was used. Bands were identified based on molecular weight using the protein marker (Precision Plus Protein™ Dual Color Standards, 1610374, Biorad) as a reference. Intensities were quantified using Image J software (National Institutes of Health, Bethesda, Md., USA).

Statistical Analysis

Statistical calculation was performed using GraphPad Prism 5.0 software (GraphPad Software, La Jolla, Calif., USA). Data were expressed as mean±standard error of mean (SEM). The statistical significance of the differences between the two groups was calculated by unpaired Student's t-test, α=0.05. When three experimental groups were compared, one-way ANOVA followed by Tukey's post-hoc test (α=0.05) was used. For corneal wound closure and corneal opacity analysis, two-way ANOVA was performed, considering p<0.05 statistically significant.

Central Wound Model

Eight-week-old male mice C57BL6/N (Charles River, Calco, Italy) (WT mice, n=10) and Tac1-deficient B6.Cg-Tac1tm1Bbm/J (Jackson Laboratories, Bar Harbor, Me., USA) (TAC1-KO mice, n=10) were employed. Animals were anesthetized with an intraperitoneal injection of tribromoethanol (250 mg/kg) and a topical anesthetic was applied to their ocular surface prior to the surgical procedure. A central circular corneal epithelial defect (2 mm) was made with a blunt spatula. To evaluate wound healing, in vivo corneal fluorescein staining was used and pictures were taken every day under the blue light. The percentage of green fluorescent area on the total corneal area was evaluated by analyzing the images by Image J software (National Institutes of Health, Bethesda, Md., USA).

Evaluation of ATP-Binding Cassette Sub-Family B Member 5 (ABCB5) Expression

The expression of ABCB5 was assayed by immunofluorescence and Western Blot. For immunofluorescence, sample preparation as well as immunostaining were previously described in section 2.8. The primary antibody used was a rabbit anti-ABCB5 (NBP1-77687, Novus Biological), followed by AlexaFluor 488 donkey anti-rabbit IgG, diluted in 2% BSA, 0.3% Triton X-100, and 0.05% Tween-20. The number of ABCB5+ cells was quantified using Image J software as described above (National Institutes of Health, Bethesda, Md., USA).

For Western Blot analysis, corneal epithelium was isolated after 30 minutes of EDTA treatment (Sigma-Aldrich Corp., St. Louis, Mo., USA) at 37° C. A pool of four corneal epithelia from different animals was used. A sample of 10 μg of proteins from WT or TAC1-KO epithelia was re-suspended RIPA Buffer (Sigma-Aldrich) containing protease inhibitor, resolved on NuPAGE 4-12% Bis-Tris Protein Gels (Thermo Fisher) and electro-transferred to nitrocellulose membranes (Amersham, Little Chalfont, UK). Red Ponceau staining (Sigma-Aldrich) was used to evaluate protein transfer. Membranes were blocked in a Tris-buffered solution (TBS) 3% BSA, 0.1% Tween 20 and incubated overnight with rabbit anti-ABCB5 (AP6122a-ev, Abcepta) at 4° C. under gentle shaking. Subsequently, membranes were incubated at RT for 1 h with HRP-conjugated secondary antibodies (NA9340V, Ge Healthcare) followed by chemiluminescence reaction performed with ECL detection reagent (Ge Healthcare). The chemiluminescent blots were imaged with ChemiDoc (Bio-Rad). Expression of β-actin HRP conjugated mouse monoclonal antibody (A3854, Merck) was used as loading control. Bands were selected based on the expected molecular weight using a molecular weight marker and intensities were quantified using Image J software (National Institutes of Health, Bethesda, Md., USA).

Results

Substance P Ablation and NK1R Blockade Improves Corneal Epithelial Wound Healing and Corneal Transparency

After disepithelization, in vivo corneal photographs were taken every day in order to evaluate wound healing progress and corneal opacity in WT and TAC1-KO mice (FIG. 2). When compared to WT, TAC1-KO mice showed significantly faster epithelial wound healing, starting from the third day after disepithelization. By day 14, wound healing was 57% higher in TAC1-KO mice (p<0.001), as it is shown by the reduced fluorescein staining (FIG. 2, A). Of note, when a central less severe wound was performed, both WT and TAC1-KO mice showed a wound closure of 100% by day seven (FIG. 9), suggesting that SP is at play in a stem cell deficiency setting.

Additionally, TAC1-KO mice presented a significant increase in corneal transparency compared to WT mice (FIG. 2, B). By day 14, corneal transparency was 70% higher in TAC1-KO mice than in WT (p<0.001).

In order to test if SP was acting through NK1R binding, inventors used the selective NK1R antagonist Fosaprepitant in two different concentrations: 0.1 mg/mL (FOSA 0.1) and 1 mg/mL (FOSA 1). When wound healing rate was evaluated, inventors found that FOSA 1 mice showed a significant improvement in wound closure rate from day five when compared to WT mice (p<0.05) (FIG. 5, A). In addition, with Fosaprepitant 1 mg/mL treatment, a better corneal transparency was achieved (p<0.05) (FIG. 5, B). Mice treated with Fosaprepitant 0.1 m/mL showed no improvement in wound closure or corneal opacity compared to WT mice. These results suggest that SP-NK1R binding is detrimental for epithelial wound closure and corneal transparency in stem cell deficiency.

Goblet and Conjunctival Cells Infiltrating the Cornea are Reduced in the Absence of Substance P or by Blocking NK1R

PAS staining was employed to identify goblet cells in the cornea [Ferrari G, Bignami F, Giacomini C, Franchini S, Rama P. Safety and efficacy of topical infliximab in a mouse model of ocular surface scarring. Investig Ophthalmol Vis Sci 2013; 54:1680-8.

https://doi.org/10.1167/iovs.12-10782]. Representative images (40×) are shown in FIG. 6, A. TAC1-KO and FOSA 1 mice corneas presented a decrease of PAS+ cells when compared to WT (p<0.05).

To evaluate conjunctival cells infiltrating the cornea, CK8 and CK19 staining was performed. In addition, CK12 was used to stain the corneal epithelium. Representative images (20×) of the immunofluorescence are shown in FIG. 6, B and C. When compared to WT, TAC1-KO and FOSA 1 mice showed a reduction in both CK8 (p<0.05 and p<0.01) and CK19 (p<0.001 and p<0.05) conjunctival cells with an increase in CK12 expression (p<0.001). These results suggest that in stem cell deficiency SP promotes conjunctival migration into the cornea by binding to NK1R.

Substance P Ablation and NK1R Blockade Alleviates Stem Cell Deficiency

Using the widely accepted slow-cycling characteristic of stem cells, inventors identified corneal epithelial stem cells using BrdU [Cotsarelis G, Cheng S Z, Dong G, Sun T T, Lavker R M. Existence of slow-cycling limbal epithelial basal cells that can be preferentially stimulated to proliferate: Implications on epithelial stem cells. Cell 1989; 57:201-9. https://doi.org/10.1016/0092-8674(89)90958-6; Sartaj R, Zhang C, Wan P, Pasha Z, Guaiquil V, Liu A, et al. Characterization of slow cycling corneal limbal epithelial cells identifies putative stem cell markers. Sci Rep 2017; 7. https://doi.org/10.1038/s41598-017-04006-y; Pajoohesh-Ganji A, Pal-Ghosh S, Tadvalkar G, Stepp M A. Corneal goblet cells and their niche: Implications for corneal stem cell deficiency. Stem Cells 2012; 30:2032-43. https://doi.org/10.1002/stem.1176; Li J, Xiao Y, Coursey T G, Chen X, Deng R, Lu F, et al. Identification for Differential Localization of Putative Corneal Epithelial Stem Cells in Mouse and Human. Sci Rep 2017; 7. https://doi.org/10.1038/s41598-017-04569-w] in addition to the recognized stem cell marker p63 [Pellegrini G, Dellambra E, Golisano O, Martinelli E, Fantozzi I, Bondanza S, et al. p63 identifies keratinocyte stem cells. Proc Natl Acad Sci USA 2001; 98:3156-61. https://doi.org/10.1073/pnas.061032098]. Inventors found that both TAC1-KO and FOSA 1 mice presented an increase in BrdU staining, when compared to WT (p<0.01), while no differences were found in p63 content (FIG. 7, A). Inventors then assessed the colocalization of the two makers and both groups displayed higher percentage of p63/BrdU colocalization (p<0.05), which was located mostly in the basal epithelium (FIG. 7, B). Finally, inventors identified and quantified stem cell-like cells as the cells presenting more than 50% of the nucleus positive for both p63 and BrdU. In line with the previous results, inventors found that TAC1-KO and FOSA 1 mice also exhibited an increased number of stem cell-like cells (p<0.01) compared to WT (FIG. 7, B). In addition, inventors also quantified the expression of ATP-binding cassette sub-family B member 5 (ABCB5), recently described as another putative marker of epithelial stem cells [Ksander B R, Kolovou P E, Wilson B J, Saab K R, Guo Q, Ma J, et al. ABCB5 is a limbal stem cell gene required for corneal development and repair. Nature 2014; 511:353-7. https://doi.org/10.1038/nature13426]. TAC1-KO mice exhibited an increase in ABCB5 expression in the corneal epithelium (19%, p<0.05) (FIG. 10).

Inventors then evaluated if corneal epithelial stem cells also expressed NK1R, as it has been already demonstrated in corneal epithelial cells [Watanabe M, Nakayasu K, Iwatsu M, Kanai A. Endogenous substance P in corneal epithelial cells and keratocytes. Jpn J Ophthalmol 2002; 46:616-20. https://doi.org/10.1016/S0021-5155(02)00617-2]. As shown in FIG. 7, C, corneal epithelial stem cells are also NK1R positive. Altogether, these results suggest that after a severe wound stem cells are preserved in the absence of SP or after blocking NK1R.

Substance P Ablation Results in Decreased mTOR Signaling and Senescence in Corneal Epithelium

The ribosomal protein S6 kinase is a well-known downstream target of mTOR related to cell growth, and its phosphorylation has been used as a hallmark of mTOR pathway activation [Magnuson B, Ekim B, Fingar D C. Regulation and function of ribosomal protein S6 kinase (S6K) within mTOR signalling networks. Biochem J 2012; 441:1-21. https://doi.org/10.1042/BJ20110892]. The expression of P70S6 and pP70S6 was evaluated by western blot. A representative blot is shown in FIG. 8, A. Bands of approximately 70 kDa and 40 kDa correspond to pP70S6 or P70S6 and β-actin, respectively. When compared to WT, TAC1-KO mice showed a significant decrease in pP70S6 expression (51%, respectively, p<0.05). A ratio between pP70S6 and P70S6 expression was performed in order to determine mTOR pathway activation. A decrease in this ratio was observed in TAC1-KO mice, compared to WT, suggesting an inhibition of mTOR signaling pathway.

Finally, γH2AX expression was assayed as a senescence marker and as a target downregulated by mTOR pathway. A representative blot is shown in FIG. 8, B. Bands of approximately 15 kDa and 40 kDa correspond to γH2AX and β-actin, respectively. TAC1-KO mice displayed a decreased γH2AX expression in comparison to WT (53%, p<0.05), suggesting that cell senescence is prevented when SP is ablated.

Discussion

Epithelial stem cells are indispensable to repopulate the cornea after injury and to prevent vision loss [Dua H S, Azuara-Blanco A. Limbal stem cells of the corneal epithelium. Surv Ophthalmol 2000; 44:415-25. https://doi.org/10.1016/30039-6257(00)00109-0; Dua H S, Saini J S, Azuara-Blanco A, Gupta P. Limbal stem cell deficiency: Concept, aetiology, clinical presentation, diagnosis and management. Indian J Ophthalmol 2000; 48:83-92Daniels J T, Harris A R, Mason C. Corneal epithelial stem cells in health and disease. Stem Cell Rev 2006; 2:247-54. https://doi.org/10.1007/s12015-006-0053-4].

Many ocular diseases including corneal burns, severe dry eye, and autoimmune disorders can induce SCD [Hatch K M, Dana R. The structure and function of the limbal stem cell and the disease states associated with limbal stem cell deficiency. Int Ophthalmol Clin 2009; 49:43-52. https://doi.org/10.1097/110.0b013e3181924e54, Ahmad S. Concise Review: Limbal Stem Cell Deficiency, Dysfunction, and Distress. Stem Cells Transl Med 2012; 1:110-5. https://doi.org/10.5966/sctm.2011-0037; Daniels J T, Dart J K G, Tuft S J, Khaw P T. Corneal stem cells in review. Wound Repair Regen 2001; 9:483-94. https://doi.org/10.1046/j.1524-475x.2001.00483.x.]. Inventors herein report the beneficial effect of Substance P (SP) ablation or pharmacological inhibition of its principal receptor (NK1R), on wound healing efficiency in a well-established mouse model of epithelial stem cell deficiency [Amirjamshidi H, Milani B Y, Sagha H M, Movahedan A, Shafiq M A, Lavker R M, et al. Limbal fibroblast conditioned media: A non-invasive treatment for limbal stem cell deficiency. Mol Vis 2011; 17:658, Pal-Ghosh S, Tadvalkar G, Jurjus R A, Zieske J D, Stepp M A. BALB/c and C57BL6 mouse strains vary in their ability to heal corneal epithelial debridement wounds. Exp Eye Res 2008; 87:478-86. https://doi.org/10.1016/j.exer.2008.08.013; Meyer-Blazejewska E A, Call M K, Yamanaka O, Liu H, Schlötzer-Schrehardt U, Kruse F E, et al. From hair to cornea: Toward the therapeutic use of hair follicle-derived stem cells in the treatment of limbal stem cell deficiency. Stem Cells 2011; 29:57-66. https://doi.org/10.1002/stem.550]. Inventors found that activation of the SP/NK1R pathway promotes corneal opacity, conjunctival/goblet cell infiltration, and stem cell exhaustion. Specifically, SP aggravates corneal SCD through stimulation of the mTOR pathway, eventually leading to accelerated corneal epithelial cell senescence. In addition, inventors showed for the first time that corneal epithelial stem cells express NK1R and, hence, they are directly susceptible to SP effects.

It is a well-known fact that chronic inflammation can induce depletion of stem cells in bone marrow [King K Y, Goodell M A. Inflammatory modulation of HSCs: Viewing the HSC as a foundation for the immune response. Nat Rev Immunol 2011; 11:685-92. https://doi.org/10.1038/nri3062], brain [Pluchino S, Muzio L, Imitola J, Deleidi M, Alfaro-Cervello C, Salani G, et al. Persistent inflammation alters the function of the endogenous brain stem cell compartment. Brain 2008; 131:2564-78. https://doi.org/10.1093/brain/awn198; Glass C K, Saijo K, Winner B, Marchetto M C, Gage F H. Mechanisms Underlying Inflammation in Neurodegeneration. Cell 2010; 140:918-34. https://doi.org/10.1016/j.cell.2010.02.016], and hair follicles [Doles J, Storer M, Cozzuto L, Roma G, Keyes W M. Age-associated inflammation inhibits epidermal stem cell function. Genes Dev 2012; 26:2144-53. https://doi.org/10.1101/gad.192294.112], among other tissues [Kizil C, Kyritsis N, Brand M. Effects of inflammation on stem cells: together they strive? EMBO Rep 2015; 16:416-26. https://doi.org/10.15252/embr.201439702]. Interestingly, severe and/or chronic inflammation of the ocular surface invariably accompanies SCD in humans [Dua H S, Saini J S, Azuara-Blanco A, Gupta P. Limbal stem cell deficiency: Concept, aetiology, clinical presentation, diagnosis and management. Indian J Ophthalmol 2000; 48:83-92], which is why these patients require long-term topical or systemic immunosuppression (e.g: corticosteroids). On the other hand, recalcitrant ocular surface inflammation is the most common cause of autologous corneal epithelial stem cell transplantation failure [Rama P, Matuska S, Paganoni G, Spinelli A, De Luca M, Pellegrini G. Limbal stem-cell therapy and long-term corneal regeneration. N Engl J Med 2010; 363:147-55. https://doi.org/10.1056/NEJMoa0905955], which suggests that inflammation can not only induce SCD, but also prevent engraftment of transplanted stem cells. Substance P is a potent pro-inflammatory molecule that promotes neovascularization [Bignami F, Lorusso A, Rama P, Ferrari G. Growth inhibition of formed corneal neovascularization following Fosaprepitant treatment. Acta Ophthalmol 2017; 95:e641-8. https://doi.org/10.1111/aos.13304, Ziche M, Morbidelli L, Pacini M, Geppetti P, Alessandri G, Maggi C A. Substance P stimulates neovascularization in vivo and proliferation of cultured endothelial cells. Microvasc Res 1990; 40:264-78. https://doi.org/10.1016/0026-2862(90)90024-L], leukocyte chemotaxis and oxidative burst [Mashaghi A, Marmalidou A, Tehrani M, Grace P M, Pothoulakis C, Dana R. Neuropeptide substance P and the immune response. Cell Mol Life Sci 2016; 73:4249-64. https://doi.org/10.1007/s00018-016-2293-z, Suvas S. Role of Substance P Neuropeptide in Inflammation, Wound Healing, and Tissue Homeostasis. J Immunol 2017; 199:1543-52. https://doi.org/10.4049/jimmunol.1601751] in the cornea, although its role in SCD remained elusive.

In order to test the effect of SP on stem cell function, inventors decided to use an established animal model of SCD [Atallah M R, Palioura S, Perez V L, Amescua G. Limbal stem cell transplantation: Current perspectives. Clin Ophthalmol 2016; 10:593-602. https://doi.org/10.2147/OPTH.S83676, Pal-Ghosh S, Tadvalkar G, Jurjus R A, Zieske J D, Stepp M A. BALB/c and C57BL6 mouse strains vary in their ability to heal corneal epithelial debridement wounds. Exp Eye Res 2008; 87:478-86. https://doi.org/10.1016/j.exer.2008.08.013; Meyer-Blazejewska E A, Call M K, Yamanaka O, Liu H, Schlötzer-Schrehardt U, Kruse F E, et al. From hair to cornea: Toward the therapeutic use of hair follicle-derived stem cells in the treatment of limbal stem cell deficiency. Stem Cells 2011; 29:57-66. https://doi.org/10.1002/stem.550]. This is generated through total removal of corneal epithelium, including the limbus. Interestingly, inventors found that SP ablation or pharmacological inhibition of NK1R by means of Fosaprepitant improved clinical outcomes such as epithelial wound size, conjunctivalization and corneal opacity in this model.

Multiple markers of epithelial stem cells in the mouse cornea have been proposed, although a definitive consensus has not been reached yet [Guo Z H, Zhang W, Jia Y Y S, Liu Q X, Li Z F, Lin J S. An insight into the difficulties in the discovery of specific biomarkers of limbal stem cells. Int J Mol Sci 2018; 19:1982. https://doi.org/10.3390/ijms19071982]. Among them, BrdU-based pulse-chase experiments have been extensively used to identify corneal stem cells due to their slow cycling nature [Cotsarelis G, Cheng S Z, Dong G, Sun T T, Lavker R M. Existence of slow-cycling limbal epithelial basal cells that can be preferentially stimulated to proliferate: Implications on epithelial stem cells. Cell 1989; 57:201-9. https://doi.org/10.1016/0092-8674(89)90958-6; Sartaj R, Zhang C, Wan P, Pasha Z, Guaiquil V, Liu A, et al. Characterization of slow cycling corneal limbal epithelial cells identifies putative stem cell markers. Sci Rep 2017; 7. https://doi.org/10.1038/541598-017-04006-y; Pajoohesh-Ganji A, Pal-Ghosh S, Tadvalkar G, Stepp M A. Corneal goblet cells and their niche: Implications for corneal stem cell deficiency. Stem Cells 2012; 30:2032-43. https://doi.org/10.1002/stem.1176; Li J, Xiao Y, Coursey T G, Chen X, Deng R, Lu F, et al. Identification for Differential Localization of Putative Corneal Epithelial Stem Cells in Mouse and Human. Sci Rep 2017; 7. https://doi.org/10.1038/s41598-017-04569-w, Park J W, Ko J H, Kim B H, Ryu J S, Kim H J, Kim M K, et al. Inhibition of mTOR by Rapamycin Aggravates Corneal Epithelial Stem Cell Deficiency by Upregulating Inflammatory Response. Stem Cells 2019; 37:1212-22. https://doi.org/10.1002/stem.3036]. Furthermore, it has been shown that BrdU positive cells also express other putative stem cell markers such as β1 and β4-integrins [Pajoohesh-Ganji A, Pal-Ghosh S, Simmens S J, Stepp M A. Integrins in Slow-Cycling Corneal Epithelial Cells at the Limbus in the Mouse. Stem Cells 2006; 24:1075-86. https://doi.org/10.1634/stemcells.2005-0382], NGF [Li J, Xiao Y, Coursey T G, Chen X, Deng R, Lu F, et al. Identification for Differential Localization of Putative Corneal Epithelial Stem Cells in Mouse and Human. Sci Rep 2017; 7. https://doi.org/10.1038/s41598-017-04569-w], ABCB5 [Ksander B R, Kolovou P E, Wilson B J, Saab K R, Guo Q, Ma J, et al. ABCB5 is a limbal stem cell gene required for corneal development and repair. Nature 2014; 511:353-7. https://doi.org/10.1038/nature13426], and p63 [Lee H K, Ryu J S, Jeong H J, Kim M K, Oh J Y. Protection of Corneal Limbus from Riboflavin Prevents Epithelial Stem Cell Loss after Collagen Cross-Linking. J Ophthalmol 2018; 2018. https://doi.org/10.1155/2018/6854298].

In this study, inventors used two widely recognized markers such as BrdU-label retention and p63 to identify corneal epithelial stem cell. In addition, inventors also quantified the expression of ABCB5.

Inventors acknowledge that not every slow-cycling (BrdU+) cell nor all p63 positive cells are epithelial stem cells. Therefore, inventors quantified epithelial stem cells by co-localization of both markers: BrdU, located in most part of the nucleus [Pajoohesh-Ganji A, Pal-Ghosh S, Simmens S J, Stepp M A. Integrins in Slow-Cycling Corneal Epithelial Cells at the Limbus in the Mouse. Stem Cells 2006; 24:1075-86. https://doi.org/10.1634/stemcells.2005-0382], and p63. The present data suggest that SP/NK1R pathway has a role in stem cell exhaustion, and that ablation of SP or its pharmacological inhibition ameliorates stem cell deficiency. Interestingly, inventors showed for the first time that NK1R is expressed on the cell membrane of corneal epithelial stem cells, supporting the key role of SP (and nerves) in stem cell pathophysiology.

It should be noted that previous works suggest a role for SP in promoting wound healing in the cornea [Nagano T, Nakamura M, Nakata K, Yamaguchi T, Takase K, Okahara A, et al. Effects of substance P and IGF-1 in corneal epithelial barrier function and wound healing in a rat model of neurotrophic keratopathy. Investig Ophthalmol Vis Sci 2003; 44:3810-5. https://doi.org/10.1167/iovs.03-0189, Nakamura M, Ofuji K, Chikama T I, Nishida T. Combined effects of substance P and insulin-like growth factor-1 on corneal epithelial wound closure of rabbit in vivo. Curr Eye Res 1997; 16:275-8. https://doi.org/10.1076/ceyr.16.3.275.15409, Yang L, Di G, Qi X, Qu M, Wang Y, Duan H, et al. Substance P promotes diabetic corneal epithelial wound healing through molecular mechanisms mediated via the neurokinin-1 receptor. Diabetes 2014; 63:4262-74. https://doi.org/10.2337/db14-0163], although the specific effect of SP on epithelial stem cell was not studied. In this vein, inventors did not observe delayed epithelial healing in vivo, either in case of total or partial epithelial ablation, in the knock-out animal model. In addition, it has been suggested that the effect of SP on epithelial wound closure is time and concentration-dependent [Hong H S, Lee J, Lee E, Kwon Y S, Lee E, Ahn W, et al. A new role of substance P as an injury-inducible messenger for mobilization of CD29+stromal-like cells. Nat Med 2009; 15:425-35. https://doi.org/10.1038/nm.1909]. Hence, it is tempting to hypothesize that an early, controlled release of SP is beneficial, while excessive amounts may impair healing. A similar effect was observed in SP-cultured vascular endothelial cells, where cell proliferation was stimulated at low SP concentration, but impaired at higher ones [Villablanca A C, Murphy C J, Reid T W. Growth-promoting effects of substance P on endothelial cells in vitro: Synergism with calcitonin gene-related peptide, insulin, and plasma factors. Circ Res 1994; 75:1113-20. https://doi.org/10.1161/01.RES.75.6.1113]. In addition, previous data suggest that severe corneal injury increases the expression of SP locally, promotes leukocyte infiltration, and neovascularization [Bignami F, Giacomini C, Lorusso A, Aramini A, Rama P, Ferrari G. NK1 receptor antagonists as a new treatment for corneal neovascularization. Invest Ophthalmol Vis Sci 2014; 55:6783-94. https://doi.org/10.1167/iovs.14-14553]. Therefore, such an increase of SP favors inflammation and accelerate senescence and depletion of stem cells, with consequent infiltration of conjunctival cells.

A potential mechanism inducing corneal epithelial stem cell senescence is the target of rapamycin (mTOR) signaling, which controls aging, cellular senescence, and lifespan by regulation of stem cell proliferation and differentiation in the oral and intestinal mucosa, skin, and cornea [Gidfar S, Milani F Y, Milani B Y, Shen X, Eslani M, Putra I, et al. Rapamycin Prolongs the Survival of Corneal Epithelial Cells in Culture. Sci Rep 2017; 7. https://doi.org/10.1038/srep40308, Weichhart T. mTOR as Regulator of Lifespan, Aging, and Cellular Senescence: A Mini-Review. Gerontology 2018; 64:127-34. https://doi.org/10.1159/000484629, Iglesias-Bartolome R, Patel V, Cotrim A, Leelahavanichkul K, Molinolo A A, Mitchell J B, et al. mTOR inhibition prevents epithelial stem cell senescence and protects from radiation-induced mucositis. Cell Stem Cell 2012; 11:401-14. https://doi.org/10.1016/j.stem.2012.06.007, Castilho R M, Squarize C H, Chodosh L A, Williams B O, Gutkind J S. mTOR Mediates Wnt-Induced Epidermal Stem Cell Exhaustion and Aging. Cell Stem Cell 2009; 5:279-89. https://doi.org/10.1016/j.stem.2009.06.017]. Interestingly, the effects of mTOR activation are double-faced, depending on duration. Short term activation of mTOR is beneficial, since it increases cell proliferation [Saxton R A, Sabatini D M. mTOR Signaling in Growth, Metabolism, and Disease. Cell 2017; 168:960-76. https://doi.org/10.1016/j.cell.2017.02.004]. Long-term activation, instead, leads to stem cell exhaustion and its inhibition preserves adult stem cell function [Johnson S C, Rabinovitch P S, Kaeberlein M. MTOR is a key modulator of ageing and age-related disease. Nature 2013; 493:338-45. https://doi.org/10.1038/nature11861]. DNA damage is also a major cause of stem cell senescence and exhaustion in several tissues [Chitikova Z V., Gordeev S A, Bykova T V., Zubova S G, Pospelov V A, Pospelova T V. Sustained activation of DNA damage response in irradiated apoptosis-resistant cells induces reversible senescence associated with mTOR downregulation and expression of stem cell markers. Cell Cycle 2014; 13:1424-39. https://doi.org/10.4161/cc.28402, Wang J, Sun Q, Morita Y, Jiang H, Groß A, Lechel A, et al. A differentiation checkpoint limits hematopoietic stem cell self-renewal in response to DNA damage. Cell 2012; 148:1001-14. https://doi.org/10.1016/j.cell.2012.01.040, Yu Q, Katlinskaya Y V., Carbone C J, Zhao B, Katlinski K V., Zheng H, et al. DNA-Damage-Induced Type I Interferon Promotes Senescence and Inhibits Stem Cell Function. Cell Rep 2015; 11:785-97. https://doi.org/10.1016/j.celrep.2015.03.069]. Interestingly, restraining mTOR activation suppresses cellular senescence in several tissues [Iglesias-Bartolome R, Patel V, Cotrim A, Leelahavanichkul K, Molinolo A A, Mitchell J B, et al. mTOR inhibition prevents epithelial stem cell senescence and protects from radiation-induced mucositis. Cell Stem Cell 2012; 11:401-14. https://doi.org/10.1016/j.stem.2012.06.007, Castilho R M, Squarize C H, Chodosh L A, Williams B O, Gutkind J S. mTOR Mediates Wnt-Induced Epidermal Stem Cell Exhaustion and Aging. Cell Stem Cell 2009; 5:279-89. https://doi.org/10.1016/j.stem.2009.06.017, Demidenko Z N, Zubova S G, Bukreeva E I, Pospelov V A, Pospelova T V, Blagosklonny M V. Rapamycin decelerates cellular senescence. Cell Cycle 2009; 8:1888-95. https://doi.org/10.4161/cc.8.12.8606], and specifically inhibits H2AX histone phosphorylation [Pospelova T V., Demidenko Z N, Bukreeva E I, Pospelov V A, Gudkov A V., Blagosklonny M V. Pseudo-DNA damage response in senescent cells. Cell Cycle 2009; 8:4112-8. https://doi.org/10.4161/cc.8.24.10215]. In this sense, H2AX is an early marker of DNA damage; its phosphorylation follows genotoxic stress and plays an essential role in the recruitment of DNA repair proteins [Redon C, Pilch D, Rogakou E, Sedelnikova O, Newrock K, Bonner W. Histone H2A variants H2AX and H2AZ. Curr Opin Genet Dev 2002; 12:162-9. https://doi.org/10.1016/50959-437X(02)00282-4]. Our data show that the release of SP in the highly pro-inflammatory SCD model induces mTOR expression and promotes cellular senescence through H2AX phosphorylation, supporting a role for excessive SP expression in corneal stem cell exhaustion. In this vein, the previous finding that mTOR inhibition prolongs the survival of corneal epithelial cells in vitro and maintains their proliferative potential corroborates our results [Gidfar S, Milani F Y, Milani B Y, Shen X, Eslani M, Putra I, et al. Rapamycin Prolongs the Survival of Corneal Epithelial Cells in Culture. Sci Rep 2017; 7. https://doi.org/10.1038/srep40308]. Moreover, the recent observation that mTOR signaling inhibition accelerates corneal healing following alkali burn [Wang Y, Gao G, Wu Y, Wang Y, Wu X, Zhou Q. S100A4 Silencing Facilitates Corneal Wound Healing After Alkali Burns by Promoting Autophagy via Blocking the P13K/Akt/mTOR Signaling Pathway. Invest Ophthalmol Vis Sci 2020; 61:19. https://doi.org/10.1167/iovs.61.11.19] is in line with our results. Interestingly, mTOR also plays a key role in regulating scarring, neovascularization, and inflammation in the cornea [Shin Y J, Hyon J Y, Choi W S, Yi K, Chung E S, Chung T Y, et al. Chemical injury-induced corneal opacity and neovascularization reduced by rapamycin via TGF-β1/ERK pathways regulation. Investig Ophthalmol Vis Sci 2013; 54:4452-8. https://doi.org/10.1167/iovs.13-11684, Lee K S, Ko D A, Kim E S, Kim M J, Tchah H, Kim J Y. Bevacizumab and rapamycin can decrease corneal opacity and apoptotic keratocyte number following photorefractive keratectomy. Investig Ophthalmol Vis Sci 2012; 53:7645-53. https://doi.org/10.1167/iovs.12-10494]. On the other hand, others [Park J W, Ko J H, Kim B H, Ryu J S, Kim H J, Kim M K, et al. Inhibition of mTOR by Rapamycin Aggravates Corneal Epithelial Stem Cell Deficiency by Upregulating Inflammatory Response. Stem Cells 2019; 37:1212-22. https://doi.org/10.1002/stem.3036] have shown that mTOR inhibitor rapamycin aggravates corneal epithelial stem cell deficiency by upregulating the inflammatory response. An explanation for this apparent discrepancy is that rapamycin almost completely inhibits mTOR by blocking substrate recruitment [Waldner M, Fantus D, Solari M, Thomson A W. New perspectives on mTOR inhibitors (rapamycin, rapalogs and TORKinibs) in transplantation. Br J Clin Pharmacol 2016; 82:1158-70. https://doi.org/10.1111/bcp.12893], which results in an imbalanced pro-inflammatory environment, detrimental to stem cell function. Instead, SP ablation promotes a partial inhibition of mTOR activity (around 50%), which exerts beneficial effects on epithelial cell survival and proliferation.

SCD is a major clinical problem and an area of unmet medical need. While cell transplantation therapies are now available, they cannot be accessed by everyone in need, and chronic inflammation significantly impairs their efficacy. Inventors suggest that the pro-inflammatory mediator SP can favor SCD by inducing cell senescence through mTOR signaling. Therefore, inhibition of SP activity by means of topical NK1R antagonists represents an attractive option to treat corneal epithelial stem cell deficiency and/or improve success of epithelial stem cell transplantation

The present data suggest that hyper-expression of SP can induce senescence and exhaustion of residual stem cells through activation of NK1R. Inventors anticipate that our findings have relevant translational implications, because clinically available NK1R antagonists can be repurposed for ocular use.

### Example 3

Characterization of Acute Ocular Graft-Versus-Host Disease in a Novel Preclinical Mouse Model

Materials and Methods

Animals

10-week-old male BALB/C and C57BL/6N mice (Charles River Laboratory, Lecco, Italy) were used for all the experiments (n=5/each group). All mice were free from ocular surface disease at baseline. The housing conditions were controlled (temperature 22° C., light from 07:00-19:00, humidity 50%-60%), and fresh food and water were freely available. The animals were monitored 3 times per week for 28 days post-transplantation for in vivo slit-lamp microscopy imaging, ocular surface score and systemic GVHD score (FIG. 11A). Carbon dioxide inhalation and subsequent cervical dislocation were applied to euthanize the animals. All the experiments were approved by the Animal Care and Use Committee of the IRCCS San Raffaele Scientific Institute, in accordance with the ARVO Statement for the Use of Animals in Ophthalmic and Vision Research.

Hematopoietic Stem Cell Transplantation

Allogenic BMT was conducted using the C57BL/6N and BALB/c mice as transplant donors and recipients, respectively. The recipient mice received total body irradiation (TBI) (900 cGy divided in two fractions) from a Cs source 1 day before the transplantation. The donor C57BL/6N mice were sacrificed on dry ice and they femur and spleen were harvested. The bones were gently crushed manually using a sterile stick, and bone marrow (BM) cells were released by mixing the fragments of bones with RPMI. The suspension of BM cells was filtered to remove the impurities inside. The spleens were also gently crushed into a cell suspension and filtered. Inventors suspended the BM cells and splenic cells with RPMI and adjusted the density of cells to 1×10⁶/100 mL and 2×10⁶/100 mL, respectively. Each animal received an infusion of 2×10⁶ C57BL/6N allogeneic donor BM cells intravenously with or without splenocytes (each animal received 100 μL, 2×10⁶ cells intravenously) as a source of allogeneic T cells (BM and BM+T groups).

Fosaprepitant Treatment

A group of BM+T cells mice topically received Fosaprepitant 10 mg/mL (IVEMEND, Merck Sharp & Dohme Ltd., Hoddesdon, UK) dissolved in phosphate-buffered saline (PBS), six times a day in both eyes up to day 29 (BM+T+Fosa group) (FIG. 11A). The treatment started 7 days after transplantation, when GVHD symptoms had appeared (FIG. 12A).

Systemic GVHD Assessment

Animals were monitored for established the onset of GVHD by clinical assessment using a modified version of a standard scoring system previously described by Ferrara et al. (2009). This system incorporates 5 clinical traits measuring the degree of systemic GVHD: posture, activity, weight loss, fur texture and skin integrity. Each clinical parameter was scored from 0 to 2, resulting in a total score that ranges from 0 to 10 (FIG. 11B).

Ocular GVHD Assessment

Clinical examination was performed to evaluate the ocular disease progression. Corneas were examined and photographs were taken under the slit-lamp microscope SL 990 (C.S.O., Florence, Italy) 3 times per week in a blinded fashion. In vivo corneal fluorescein staining was used to evaluate epithelial corneal damage. A scoring system was employed to evaluate ocular surface fibrosis, as previously described (Ferrari et al., 2013) (FIG. 11C). Eyelid score, as a proxy for blepharitis, was assessed in 4 eyelid areas: superior eyelid, inferior eyelid, medial and lateral canthus. The score was given based on fur loss severity. For each point, a number was given from 0 to 2 (0=no fur loss, 1=less than 50% fur loss and 2=more than 50% fur loss). The final eyelid score resulted from the sum of each point (FIG. 11D). Tear production was quantified with phenol red thread test (Zone-Quick; Lacrimedics, Eastsound, Wash., USA). Briefly, the threads were held and placed in the lateral cantus of the conjunctival fornix margin for 15 seconds. The length of the moistened thread was measured in both eyes and the average was used as lacrimal tear volume for comparative analysis.

Immunohistochemical Analysis

Mice were sacrificed 28 days after BM transplantation (Day 29 post irradiation) by carbon dioxide inhalation. Eyes including upper and lower lids, conjunctiva as well as lacrimal glands were collected and fixed with Davidson's Fixative (2% Formaldeide, 35% EtoH and 10% Glacial Acetic Acid) for 48 hours and then the samples were sectioned on a microtome at 4 μm thick and mounted on glass slide. Hematoxylin/eosin (H&E) staining was performed on corneal sections in order to evaluate parameters such as cornea thickness and integrity of corneal endothelium. Immunohistochemistry was performed using the following protocol. The sections mounted on glass slide were rehydrated in ethanol and in MilliQ water and then treated with Antigen retrieval (DIVA) for 20 min at 98° C. All sections were washed with PBS/Tween 0.05% (PBS-T) and then pretreated with 3% hydrogen peroxide in water to block endogenous peroxidase activity. Sections were subsequently washed in PBS-T and then blocked for one hour in PBS-T with 2% bovine serum albumin. The sections were then incubated one hour at room temperature with the primary antibodies goat anti mouse CD45 (1/200, AF-114, R&D Systems), goat anti mouse NK1R (1/800, ab61705, Abcam), rabbit anti mouse CD3 (1/100, ab16669, Abcam). Following three washes with PBS-T, sections were incubated in anti-goat HRP for 15 minutes and then detected with a goat-on-rodent HRP-Polymer Kit (Biocare Medical). Primary antibody labeling was detected using brown DAB substrate (Vector Laboratories, Burlingame, Calif., USA). The sections were cover slipped with Depex mounting medium (Serva, Heidelberg, Germany). Digital images of IHC-stained slides were obtained at 20× magnification using a whole slide scanner (ScanScope CS, Aperio). Images were saved in SVS format (Aperio), managed with server software (ImageServer, Aperio).

Statistical Analysis

One-way ANOVA, following Bonferroni post hoc tests, were performed to analyze the difference in systemic GVHD, corneal epithelial damage, tear secretion, phimosis, and eyelid score between groups. Unpaired t-test was applied to analyze the difference in splenocytes number, corneal thickness, CD45+ and CD3+ cells between the groups. A P value<0.05 was considered statistically significant. Results are presented as mean±standard error of the mean (SEM). All data were processed with GraphPad Prism software 6.0 (GraphPad Software, Inc., San Diego, Calif., USA).

Results

Acute GVHD Induces Epithelial Corneal Damage

Inventors first measured the systemic GVHD scores in this preclinical mouse model. Inventors found that one-week post-irradiation, animals receiving donor T cells, began to exhibit clinical signs characteristic of GVHD (Day 7 BM=2.42 vs. BM+T=4.37, p<0.0001). The systemic GVHD symptoms persisted up to day 29 post-irradiation (Day 29 BM=1.57 vs. BM+T=8.87, p<0.0001) (FIG. 12A). BM+T cells group was examined for additional phenotypes characteristic of GVHD such as the decrease of splenic cell numbers. The significant reduction of splenocytes observed in the BM+T cells group confirms the onset of GVHD in our preclinical mouse model (−240%, p<0.0001) (FIG. 12B).

To test our hypothesis that acute GVHD affects the ocular surface in this mouse model inventors analyzed the epithelial corneal damage. FIG. 12C shows representative pictures of corneal fluorescein staining at days 0, 14, 29-post-irradiation for BM and BM+T cells group. 2 weeks post-irradiation a significant increase of corneal fluorescein staining was observed only in the mice that received donor T cells (Day 14 BM=3.97 vs. BM+T cells =14.12, p<0.0001) and this epithelial damage persisted until day 29 (Day 29 BM=3.23 vs. BM+T=11.43, p<0.0001) (FIG. 12D). These results are in agreement with the onset of this disease in this preclinical mouse model. Furthermore, quantitative analysis for corneal fluorescein staining and clinical changes of GVHD demonstrated a difference in the time of induction between systemic and ocular GVHD. To further prove the epithelial corneal damage occurring in animals with GVHD, inventors performed a Hematoxylin and Eosin staining on the cornea (FIG. 12E-F). Histologic analyses demonstrated that only in the mice that developed systemic and ocular GVHD exhibited epithelial irregularity and a significant reduction on the corneal thickness (−10%, p<0.0001, FIG. 12G).

Acute GVHD Leads Several Clinical Ocular Changes

Ocular GVHD represents a disease of the ocular surface and investigating how ocular adnexa is involved in the ocular acute GVHD preclinical model should promote understanding of the mechanisms of action responsible for the development of ocular GVHD. In this frame, to confirm that ocular acute GVHD involves the ocular adnexa inventors analyzed the tear secretion, the ocular surface fibrosis, and blepharitis. Tear secretion was significantly reduced only on day 14 post-irradiation in BM+T cells group compared to BM group (BM=2.10 vs. BM+T cells=0.87, p<0.001). However, no significant difference was observed at all other time points, although a trend for reduction in tear secretion in GVHD eyes was noted (FIG. 13A). Inventors next analyzed the eyelid score as a proxy for blepharitis. Our finding indicate that the eyelid score rapidly increase in BM+T cells eyes reached significance from day 7 (BM=1.57 vs. BM+T cells=2.93, p<0.05) onwards and remained significant until day 29 post-irradiation (BM=3.5 vs. BM+T cells=5.56, p<0.001) (FIG. 13B). Finally, inventors measured the phimosis, as a proxy for ocular surface fibrosis. Our results demonstrated that in the GVHD mouse there is a significant increase on ocular surface phimosis from day 7 post-irradiation (BM=0.5 vs. BM+T cells=1.7, p<0.0001) (FIG. 13C).

These data suggest that similar to ocular GVHD occurring in the patients who undergo hematopoietic stem cell transplantation, all the structures of the ocular adnexa in our model of acute GVHD are involved and can lead to sicca and scarring.

Acute GVHD Induces the Expression of Neurokinin-1 Receptor in the Cornea

Since Substance P (SP) exerts its pro-inflammatory effects by Neurokinin-1 receptor (NK1R), inventors decided to evaluate its expression on the ocular surface and the lacrimal gland. Inventors found that BM+T cells mice showed higher levels of NK1R in the cornea (p<0.05, FIG. 14A-C) when compared to BM mice. No significant differences in NK1R expression were found in the conjunctiva and in the lacrimal gland. These results suggest that NK1R represents an attractive target to inhibit SP pro-inflammatory actions on the cornea.

Topical Fosaprepitant Reduces Epithelial Corneal Damage Induced by GVHD

Since inventors found that NK1R expression was increased in the cornea, inventors treated the mice with the NK1R antagonist Fosaprepitant to test if it was possible to avoid the corneal damage. Interestingly, inventors found that Fosaprepitant treatment significantly reduced corneal fluorescein staining from day 14, which means 7 days after the treatment started (p<0.05, FIG. 15 B). This result suggests that the SP/NK1R pathway may be involved in the pathogenesis of acute GVHD in the cornea, leading to epithelial corneal damage.

Topical Fosaprepitant Reduces Inflammatory Cell Infiltration in Acute Ocular Model of GVHD

Ocular complications in GVHD generally occurs when the donor lymphocytes attack host histocompatibility antigens, which is a T-cell mediated process that would lead to infiltration and inflammation of the conjunctiva and cornea. Based on this premise, inventors measured the inflammatory cells infiltrating in the cornea, conjunctiva as well as lacrimal gland. FIG. 16 shows representative pictures for CD45 staining in the cornea (FIG. 16A-C), conjunctival (FIG. 16E-G) and lacrimal gland (FIG. 161-M). Immunohistochemistry revealed that the BM+T cells mice showed significantly more CD45+ cells in both cornea (p<0.0002, FIG. 16D) and conjunctival (p<0.0001, FIG. 16H) than the BM mice. Interestingly, Fosaprepitant treatment significantly reduced CD45+ cell infiltration in both cornea and conjunctiva (p<0.01 and p<0.0001). Regarding the lacrimal gland, no significant difference was found in CD45+ cell infiltration among all groups.

In addition, inventors specifically examined the infiltration of CD3+ cells (T cells) in the cornea (FIG. 17A-C), conjunctiva (FIG. 17E-G), and lacrimal gland (FIG. 171-K). Immunohistochemical analysis showed that BM+T cells mice showed an increase in CD3+ cells in both cornea (p<0.0002, FIG. 16D) and conjunctiva (p<0.0001, FIG. 16F) compared to BM mice. Treatment with Fosaprepitant significantly decreased the CD3+ cells infiltrating the cornea (p<0.001, FIG. 17D), conjunctiva (p<0.001, FIG. 17H), and the lacrimal gland (p<0.001, FIG. 17L).

### REFERENCES

[1] Al-Aqaba M A, Fares U, Suleman H, Lowe J, Dua H S. Br J Ophthalmol 2010; 94:784-9. https://doi.org/10.1136/bjo.2009.173799.
[2] Shaheen B S, Bakir M, Jain S. Corneal nerves in health and disease. Surv Ophthalmol 2014; 59:263-85. https://doi.org/10.1016/j.survophthal.2013.09.002.
[3] Mashaghi A, Marmalidou A, Tehrani M, Grace P M, Pothoulakis C, Dana R. Neuropeptide substance P and the immune response. Cell Mol Life Sci 2016; 73:4249-64. https://doi.org/10.1007/s00018-016-2293-z.
[4] Watanabe M, Nakayasu K, Iwatsu M, Kanai A. Endogenous substance P in corneal epithelial cells and keratocytes. Jpn J Ophthalmol 2002; 46:616-20. https://doi.org/10.1016/S0021-5155(02)00617-2.
[5] Khawaja A M, Rogers D F. Tachykinins: Receptor to effector. Int J Biochem Cell Biol 1996; 28:721-38. https://doi.org/10.1016/1357-2725(96)00017-9.
[6] Barbariga M, Fonteyne P, Ostadreza M, Bignami F, Rama P, Ferrari G. Substance p modulation of human and murine corneal neovascularization. Investig Ophthalmol Vis Sci 2018; 59:1305-12. https://doi.org/10.1167/iovs.17-23198.
[7] Bignami F, Lorusso A, Rama P, Ferrari G. Growth inhibition of formed corneal neovascularization following Fosaprepitant treatment. Acta Ophthalmol 2017; 95:e641-8. https://doi.org/10.1111/aos.13304.
[8] Bignami F, Giacomini C, Lorusso A, Aramini A, Rama P, Ferrari G. NK1 receptor antagonists as a new treatment for corneal neovascularization. Invest Ophthalmol Vis Sci 2014; 55:6783-94. https://doi.org/10.1167/iovs.14-14553.
[9] Suvas S. Role of Substance P Neuropeptide in Inflammation, Wound Healing, and Tissue Homeostasis. J Immunol 2017; 199:1543-52. https://doi.org/10.4049/jimmunol.1601751.
[10] Amitai-Lange A, Altshuler A, Bubley J, Dbayat N, Tiosano B, Shalom-Feuerstein R. Lineage tracing of stem and progenitor cells of the murine corneal epithelium. Stem Cells 2015; 33:230-9. https://doi.org/10.1002/stem.1840.
[11] Di Girolamo N, Bobba S, Raviraj V, Delic N C, Slapetova I, Nicovich P R, et al. Tracing the fate of limbal epithelial progenitor cells in the murine cornea. Stem Cells 2015; 33:157-69. https://doi.org/10.1002/stem.1769.
[12] West J D. Evaluating alternative stem cell hypotheses for adult corneal epithelial maintenance. World J Stem Cells 2015; 7:281. https://doi.org/10.4252/wjsc.v7.i2.281.
[13] Ljubimov A V., Saghizadeh M. Progress in corneal wound healing. Prog Retin Eye Res 2015; 49:17-45. https://doi.org/10.1016/j.preteyeres.2015.07.002.
[14] Hatch K M, Dana R. The structure and function of the limbal stem cell and the disease states associated with limbal stem cell deficiency. Int Ophthalmol Clin 2009; 49:43-52. https://doi.org/10.1097/110.0b013e3181924e54.
[15] Rama P, Ferrari G, Pellegrini G. Cultivated limbal epithelial transplantation. Curr Opin Ophthalmol 2017; 28:387-9. https://doi.org/10.1097/ICU.0000000000000382.
[16] Baylis O, Figueiredo F, Henein C, Lako M, Ahmad S. 13 years of cultured limbal epithelial cell therapy: A review of the outcomes. J Cell Biochem 2011; 112:993-1002. https://doi.org/10.1002/jcb.23028.
[17] Sangwan V S, Basu S, Vemuganti G K, Sejpal K, Subramaniam S V, Bandyopadhyay S, et al. Clinical outcomes of xeno-free autologous cultivated limbal epithelial transplantation: A 10-year study. Br J Ophthalmol 2011; 95:1525-9. https://doi.org/10.1136/bjophthalmol-2011-300352.
[18] Aravena C, Bozkurt K, Chuephanich P, Supiyaphun C, Yu F, Deng S X. Classification of Limbal Stem Cell Deficiency Using Clinical and Confocal Grading. Cornea 2019; 38:1-7. https://doi.org/10.1097/ICO.0000000000001799.
[19] Na K S, Mok J W, Kim J Y, Rho C R, Joo C K. Correlations between tear cytokines, chemokines, and soluble receptors and clinical severity of dry eye disease. Investig Ophthalmol Vis Sci 2012; 53:5443-50. https://doi.org/10.1167/iovs.11-9417.
[20] Amirjamshidi H, Milani B Y, Sagha H M, Movahedan A, Shafiq M A, Lavker R M, et al. Limbal fibroblast conditioned media: A non-invasive treatment for limbal stem cell deficiency. Mol Vis 2011; 17:658.
[21] Yoeruek E, Deuter C, Gieselmann S, Saygili O, Spitzer M S, Tatar O, et al. Long-term visual acuity and its predictors after cataract surgery in patients with uveitis. Eur J Ophthalmol 2010; 20:694-701. https://doi.org/10.1177/112067211002000409.
[22] Ferrari G, Bignami F, Giacomini C, Franchini S, Rama P. Safety and efficacy of topical infliximab in a mouse model of ocular surface scarring. Investig Ophthalmol Vis Sci 2013; 54:1680-8. https://doi.org/10.1167/iovs.12-10782.
[23] Dua H S, Azuara-Blanco A. Limbal stem cells of the corneal epithelium. Surv Ophthalmol 2000; 44:415-25. https://doi.org/10.1016/S0039-6257(00)00109-0.
[24] Dua H S, Saini J S, Azuara-Blanco A, Gupta P. Limbal stem cell deficiency: Concept, aetiology, clinical presentation, diagnosis and management. Indian J Ophthalmol 2000; 48:83-92.
[25] Daniels J T, Harris A R, Mason C. Corneal epithelial stem cells in health and disease. Stem Cell Rev 2006; 2:247-54. https://doi.org/10.1007/s12015-006-0053-4.
[26] Ahmad S. Concise Review: Limbal Stem Cell Deficiency, Dysfunction, and Distress. Stem Cells Transl Med 2012; 1:110-5. https://doi.org/10.5966/sctm.2011-0037.
[27] Daniels J T, Dart J K G, Tuft S J, Khaw P T. Corneal stem cells in review. Wound Repair Regen 2001; 9:483-94. https://doi.org/10.1046/j.1524-475x.2001.00483.x.
[28] Pal-Ghosh S, Tadvalkar G, Jurjus R A, Zieske J D, Stepp M A. BALB/c and C57BL6 mouse strains vary in their ability to heal corneal epithelial debridement wounds. Exp Eye Res 2008; 87:478-86. https://doi.org/10.1016/j.exer.2008.08.013.
[29] Meyer-Blazejewska E A, Call M K, Yamanaka O, Liu H, Schlötzer-Schrehardt U, Kruse F E, et al. From hair to cornea: Toward the therapeutic use of hair follicle-derived stem cells in the treatment of limbal stem cell deficiency. Stem Cells 2011; 29:57-66. https://doi.org/10.1002/stem.550.
[30] King K Y, Goodell M A. Inflammatory modulation of HSCs: Viewing the HSC as a foundation for the immune response. Nat Rev Immunol 2011; 11:685-92. https://doi.org/10.1038/nri3062.
[31] Oh J, Lee Y D, Wagers A J. Stem cell aging: Mechanisms, regulators and therapeutic opportunities. Nat Med 2014; 20:870-80. https://doi.org/10.1038/nm.3651.
[32] Rama P, Matuska S, Paganoni G, Spinelli A, De Luca M, Pellegrini G. Limbal stem-cell therapy and long-term corneal regeneration. N Engl J Med 2010; 363:147-55. https://doi.org/10.1056/NEJMoa0905955.
[33] Ziche M, Morbidelli L, Pacini M, Geppetti P, Alessandri G, Maggi C A. Substance P stimulates neovascularization in vivo and proliferation of cultured endothelial cells. Microvasc Res 1990; 40:264-78. https://doi.org/10.1016/0026-2862(90)90024-L.
[34] Yang L, Di G, Qi X, Qu M, Wang Y, Duan H, et al. Substance P promotes diabetic corneal epithelial wound healing through molecular mechanisms mediated via the neurokinin-1 receptor. Diabetes 2014; 63:4262-74. https://doi.org/10.2337/db14-0163.
[35] Nakamura M, Ofuji K, Chikama T I, Nishida T. Combined effects of substance P and insulin-like growth factor-1 on corneal epithelial wound closure of rabbit in vivo. Curr Eye Res 1997; 16:275-8. https://doi.org/10.1076/ceyr.16.3.275.15409.
[36] Nagano T, Nakamura M, Nakata K, Yamaguchi T, Takase K, Okahara A, et al. Effects of substance P and IGF-1 in corneal epithelial barrier function and wound healing in a rat model of neurotrophic keratopathy. Investig Ophthalmol Vis Sci 2003; 44:3810-5. https://doi.org/10.1167/iovs.03-0189.
[37] Hong H S, Lee J, Lee E, Kwon Y S, Lee E, Ahn W, et al. A new role of substance P as an injury-inducible messenger for mobilization of CD29+stromal-like cells. Nat Med 2009; 15:425-35. https://doi.org/10.1038/nm.1909.
[38] Castilho R M, Squarize C H, Chodosh L A, Williams B O, Gutkind J S. mTOR Mediates Wnt-Induced Epidermal Stem Cell Exhaustion and Aging. Cell Stem Cell 2009; 5:279-89. https://doi.org/10.1016/j.stem.2009.06.017.
[39] Weichhart T. mTOR as Regulator of Lifespan, Aging, and Cellular Senescence: A Mini-Review. Gerontology 2018; 64:127-34. https://doi.org/10.1159/000484629.
[40] Iglesias-Bartolome R, Patel V, Cotrim A, Leelahavanichkul K, Molinolo A A, Mitchell J B, et al. mTOR inhibition prevents epithelial stem cell senescence and protects from radiation-induced mucositis. Cell Stem Cell 2012; 11:401-14. https://doi.org/10.1016/j.stem.2012.06.007.
[41] Johnson S C, Rabinovitch P S, Kaeberlein M. MTOR is a key modulator of ageing and age-related disease. Nature 2013; 493:338-45. https://doi.org/10.1038/nature11861.
[42] Chitikova Z V., Gordeev S A, Bykova T V., Zubova S G, Pospelov V A, Pospelova T V. Sustained activation of DNA damage response in irradiated apoptosis-resistant cells induces reversible senescence associated with mTOR downregulation and expression of stem cell markers. Cell Cycle 2014; 13:1424-39. https://doi.org/10.4161/cc.28402.
[43] Wang J, Sun Q, Morita Y, Jiang H, Groß A, Lechel A, et al. A differentiation checkpoint limits hematopoietic stem cell self-renewal in response to DNA damage. Cell 2012; 148:1001-14. https://doi.org/10.1016/j.cell.2012.01.040.
[44] Yu Q, Katlinskaya Y V., Carbone C J, Zhao B, Katlinski K V., Zheng H, et al. DNA-Damage-Induced Type I Interferon Promotes Senescence and Inhibits Stem Cell Function. Cell Rep 2015; 11:785-97. https://doi.org/10.1016/j.celrep.2015.03.069.
[45] Redon C, Pilch D, Rogakou E, Sedelnikova O, Newrock K, Bonner W. Histone H2A variants H2AX and H2AZ. Curr Opin Genet Dev 2002; 12:162-9. https://doi.org/10.1016/S0959-437X(02)00282-4.
[46] Demidenko Z N, Zubova S G, Bukreeva E I, Pospelov V A, Pospelova T V, Blagosklonny M V. Rapamycin decelerates cellular senescence. Cell Cycle 2009; 8:1888-95. https://doi.org/10.4161/cc.8.12.8606.
[47] Pospelova T V., Demidenko Z N, Bukreeva E I, Pospelov V A, Gudkov A V., Blagosklonny M V. Pseudo-DNA damage response in senescent cells. Cell Cycle 2009; 8:4112-8. https://doi.org/10.4161/cc.8.24.10215.
[48] Gidfar S, Milani F Y, Milani B Y, Shen X, Eslani M, Putra I, et al. Rapamycin Prolongs the Survival of Corneal Epithelial Cells in Culture. Sci Rep 2017; 7. https://doi.org/10.1038/srep40308.
[49] Shin Y J, Hyon J Y, Choi W S, Yi K, Chung E S, Chung T Y, et al. Chemical injury-induced corneal opacity and neovascularization reduced by rapamycin via TGF-β1/ERK pathways regulation. Investig Ophthalmol Vis Sci 2013; 54:4452-8. https://doi.org/10.1167/iovs.13-11684.
[50] Lee K S, Ko D A, Kim E S, Kim M J, Tchah H, Kim J Y. Bevacizumab and rapamycin can decrease corneal opacity and apoptotic keratocyte number following photorefractive keratectomy. Investig Ophthalmol Vis Sci 2012; 53:7645-53. https://doi.org/10.1167/iovs.12-10494.
[51] Park J W, Ko J H, Kim B H, Ryu J S, Kim H J, Kim M K, et al. Inhibition of mTOR by Rapamycin Aggravates Corneal Epithelial Stem Cell Deficiency by Upregulating Inflammatory Response. Stem Cells 2019; 37:1212-22. https://doi.org/10.1002/stem 0.3036.
[52] Waldner M, Fantus D, Solari M, Thomson A W. New perspectives on mTOR inhibitors (rapamycin, rapalogs and TORKinibs) in transplantation. Br J Clin Pharmacol 2016; 82:1158-70. https://doi.org/10.1111/bcp.12893.

1. A method for the treatment and/or prevention of stem cell deficiency, comprising administering an inhibitor of substance P and/or of its receptor to a subject in need of such treatment and/or prevention.

2. The method according to claim 1 wherein the substance P receptor is the receptor neurokinin 1 (NK1).

3. The method according to claim 1, wherein the inhibitor is an NK-1 antagonist.

4. The method according to claim 3, wherein the NK-1 antagonist is administered at a concentration of at least 0.01 mg/ml, or at least 10 mg/mL, or of at least 20 mg/mL, or of at least 30 mg/mL, or of at least 40 mg/mL, or of at least 50 mg/mL, or of at least 60 mg/mL, or of 0.01 mg/ml to 100 mg/ml.

5. The method according to claim 1, wherein the stem cell deficiency is a corneal epithelial stem cell deficiency.

6. The method according to claim 1, wherein the stem cell deficiency is selected from the group consisting of: stem cell deficiency induced by aniridia, stem cell deficiency induced by multiple endocrine deficiency, stem cell deficiency induced by epidermal dysplasia such as ectrodactyly-ectodermal dysplasia-clefting syndrome or keratitis-ichthyosis-deafness syndrome, stem cell deficiency induced by erythrokeratodermia, stem cell deficiency induced by dyskeratosis congenita, stem cell deficiency induced by thermal or chemical ocular burn, stem cell deficiency induced by inflammatory eye disease such as Stevens-Johnson syndrome, toxic epidermal necrolysis, ocular cicatricial pemphigoid, chronic limbitis (vernal/atopic conjunctivitis), stem cell deficiency induced by neurotrophic keratopathy, such as trigeminal neuralgia, diabetes, herpes simplex, zoster, stem cell deficiency induced by chronic bullous keratopathy, stem cell deficiency induced by contact lens wear, stem cell deficiency induced by neoplasia, stem cell deficiency induced by extensive limbal cryotherapy, stem cell deficiency induced by irradiation, stem cell deficiency induced by surgery involving the limbus, stem cell deficiency induced by topical antimetabolites (5-FU, mitimycin C), stem cell deficiency induced by systemic chemotherapy, stem cell deficiency induced by microbial infection extending to limbus, stem cell deficiency induced by neurotrophic keratitis, stem cell deficiency induced by vitamin A deficiency, stem cell deficiency induced by Mooren's ulcer, stem cell deficiency induced by malignancy, stem cell deficiency induced by cornea, conjunctiva, ocular surface and lid infections (including trachoma), stem cell deficiency induced by graft versus host disease (GVHD), stem cell deficiency induced by systemic medications including hydroxyurea, stem cell deficiency induced by anterior segment ischaemic syndrome, stem cell deficiency induced by KID (Keratitis Ichthyosis and Deafness syndrome).

7. The method according to claim 1, wherein the stem cell deficiency is induced by graft versus host disease.

8. The method according to any claim 1, wherein the stem cell deficiency is induced by aniridia.

9. The method according to claim 1, wherein said inhibitor improves corneal wound healing and/or improves corneal wound closure and/or inhibits at least partially mTOR.

10. (canceled)

11. The method according to claim 1, wherein the inhibitor is Fosaprepitant or a pharmaceutically acceptable salt thereof.

12. The method according to claim 3, wherein 0.01 mg/ml-100 mg/ml of NK-1 antagonist is administered.

13. The method according to claim 1, further comprising administering an agent selected from the group consisting of: an anaesthetic agent, a non-steroidal anti-inflammatory agent, an analgesic agent, an agent useful in the prevention and/or treatment of stem cell deficiency, and an agent that is used following surgery to the eye.

14. The method according to claim 1, wherein the inhibitor is administered as a topical composition.

15. The method according to claim 14, wherein the topical composition is in the form of an eye-drop formulation.

16. (canceled)

17. The method according to claim 1, wherein the stem cell deficiency is a corneal epithelial stem cell deficiency.

18. The method according to claim 1, wherein the stem cell deficiency is selected from the group consisting of: stem cell deficiency induced by aniridia, stem cell deficiency induced by multiple endocrine deficiency, stem cell deficiency induced by epidermal dysplasia such as ectrodactyly-ectodermal dysplasia-clefting syndrome or keratitis-ichthyosis-deafness syndrome, stem cell deficiency induced by erythrokeratodermia, stem cell deficiency induced by dyskeratosis congenita, stem cell deficiency induced by thermal or chemical ocular burn, stem cell deficiency induced by inflammatory eye disease such as Stevens-Johnson syndrome, toxic epidermal necrolysis, ocular cicatricial pemphigoid, chronic limbitis (vernal/atopic conjunctivitis), stem cell deficiency induced by neurotrophic keratopathy, such as trigeminal neuralgia, diabetes, herpes simplex, zoster, stem cell deficiency induced by chronic bullous keratopathy, stem cell deficiency induced by contact lens wear, stem cell deficiency induced by neoplasia, stem cell deficiency induced by extensive limbal cryotherapy, stem cell deficiency induced by irradiation, stem cell deficiency induced by surgery involving the limbus, stem cell deficiency induced by topical antimetabolites (5-FU, mitimycin C), stem cell deficiency induced by systemic chemotherapy, stem cell deficiency induced by microbial infection extending to limbus, stem cell deficiency induced by neurotrophic keratitis, stem cell deficiency induced by vitamin A deficiency, stem cell deficiency induced by Mooren's ulcer, stem cell deficiency induced by malignancy, stem cell deficiency induced by cornea, conjunctiva, ocular surface and lid infections (including trachoma), stem cell deficiency induced by graft versus host disease, stem cell deficiency induced by systemic medications including hydroxyurea, stem cell deficiency induced by anterior segment ischaemic syndrome, stem cell deficiency induced by KID (Keratitis Ichthyosis and Deafness syndrome).

19. The method according to claim 1, wherein the stem cell deficiency is induced by graft versus host disease.

20. The method according to claim 1, wherein the stem cell deficiency is induced by aniridia.

21. The method according to claim 3, wherein the NK-1 antagonist is Fosaprepitant or a pharmaceutically acceptable salt thereof.

22. The method according to claim 3, wherein t the NK-1 antagonist is administered once at a concentration of approximately 50 mg/mL, or between once and six times a day for 1 to 30 days at a concentration of 0.01 to 100 mg/ml.
